(19) United States
(12) Patent Application Publication
(10) Pub. No.: US 2014/0304845 A1
(43) Pub. Date: Oct. 9, 2014

(54) ALZHEIMER'S DISEASE SIGNATURE MARKERS AND METHODS OF USE

(71) Applicant: MERCK SHARP & DOHME CORP., Rahway, NJ (US)

(72) Inventors: Andrey Loboda, Philadelphia, PA (US); Michael Nebozhyn, Yeadon, PA (US); Alexei Podtelezhnikov, Lansdale, PA (US); David J. Stone, Wyncote, PA (US); Keith Tanis, Quakertown, PA (US); William J. Ray, Juniper, FL (US)

(21) Appl. No.: 14/354,622

(22) PCT Filed: Oct. 26, 2012
(86) PCT No.: PCT/US12/62218

§ 371 Date: Apr. 28, 2014

Related U.S. Application Data
(60) Provisional application No. 61/553,400, filed on Oct. 31, 2011.

(51) Int. Cl. C12Q 1/68; A01K 67/027

(52) U.S. Cl. CPC C12Q 1/6883; A01K 67/0275
USPC 800/12; 536/23.5; 536/23.2; 506/17

(57) ABSTRACT

Methods, biomarkers, and expression signatures are disclosed for assessing the disease progression of Alzheimer's disease (AD). In one embodiment, BioAge (biological age), NdStress (neurodegenerative stress), Alz (Alzheimer), and Inflame (inflammation) are used as biomarkers of AD progression. In another aspect, the invention comprises a gene signature for evaluating disease progression. In still another embodiment, methods for evaluating disease progression are provided. In yet another embodiment, the invention can be used to identify animal models for use in the development and evaluation of therapeutics for the treatment of AD.

### FIELD OF THE INVENTION

The invention relates generally to the use of gene expression marker gene sets that are correlated to Alzheimer's disease progression and methods of using thereof.

### BACKGROUND OF THE INVENTION

During normal aging the brain undergoes many changes resulting in a gradual but detectable cognitive decline that is associated with limited neuronal loss and glial proliferation in the cortex and gross weight decrease of 2-3% per decade (Drachman, D. A., 2006, Neurology, 67: 1340-1352; Yankner, B. A., et al., 2008, Annu. Rev. Pathol., 3:41-66). On the molecular level the mechanisms driving aging of the brain are not yet understood, but likely include mitochondrial DNA damage (Lu, T., et al., 2004, Nature 429:883-891) and chronic oxidative stress (Lin, M. T., et al., 2006, Nature 443:787-795). This slow decline in cognitive ability does not interfere with normal function through at least 100 years of life. In contrast Alzheimer's disease (AD) is a debilitating neurodegenerative disorder associated with a rapid cognitive decline with an average survival of 5-10 years after the diagnosis (Blennow, K., et al., 2006, Lancet, 368:387-403); Cummings, J. L., 2004, N. Engl. J. Med., 351:56-67; Jakob-Roetne, R. and Jacobsen, H., 2009, Angew. Chem. Int. Ed. Engl., 48:3030-3059). Age is the main AD risk factor with almost half of the population over age 85 affected. However, AD clearly differs from the normal aging in that it causes dramatic loss of synapses, neurons and brain activity in specific anatomical regions, and results in massive atrophy and gliosis (Drachman, D. A., 2006; Herrup, K., 2010, J. Neurosci., 30:16755-16762).

The factors that cause some individuals to depart from the relatively benign process of normal brain aging and instead undergo the pathological cascade that leads to AD are unknown. A number of genetic risk factors for AD have been proposed (Waring, S. C. and Rosenberg, R. N., 2008, Arch. Neurol., 65:329-334; Bertram, L. and Tanzi, R. E., 2008, Nat. Rev. Neurosci., 9:768-778; Harold, D., et al., 2009, Nat. Genet., 41:1088-1093; Lambert, J. C., et al., 2009, Nat. Genet., 41:1094-1099), however, only the apolipoprotein E (APOE) ε4-allele, which lowers the age of onset and accelerates the cognitive decline, has a large effect (Kleiman, T., et al., 2006, Dement. Geriatr. Cogn. Disord., 22:73-82; Stone, D. J., et al., 2010, Pharmacogenomics J., 10:161-164). Pathologically, AD is characterized by the presence of two insoluble protein aggregates, senile plaques formed from the peptide β-amyloid (Aβ) and neurofibrillary tangles composed of hyperphosphorylated tau protein (Goedert, M. and Spillantini, M. G., 2006, Science, 314:777-781). In rare familial AD, the cause of disease is autosomal dominant mutations in Aβ precursor protein (APP) or the Aβ-producing enzymes presenilins (PSEN1 or PSEN2), which are all thought to lead to increased levels of aggregated Aβ (Waring, S. C. and Rosenberg, R. N., 2008; Bertram, L. and Tanzi, R. E., 2008; Hardy, J. and Selkoe, D. J., 2002, Science, 297:353-356). Likewise, mutations in tau (MAPT) that predispose it to aggregation can cause specific diseases that involve profound neurodegeneration and dementia (Ballatore, C., et al., 2007, Nat. Rev. Neurosci., 8:663-672; Wolfe, M. S., 2009, J. Biol. Chem., 284: 6021-6025). Thus, like in other neurodegenerative diseases such as Huntington's disease (HD) and Parkinson's disease, the formation of toxic insoluble aggregates seems to be a key pathogenic step. It is not known why these Aβ and tau aggregates accumulate in AD patients, nor how they contribute to neuronal dysfunction, particularly as to Aβ deposits, which can often be found in the brains of elderly non-demented subjects (Schmitt, F. A., et al., 2000, Neurology, 55:370-376).

An important goal of AD research is to identify interventions that maintain brain function, potentially by inhibiting the formation or improving the clearance of neurotoxic aggregates, or by promoting resistance to or recovery from damage. A number of biological processes have been associated with AD including cholesterol metabolism, inflammation, and response to misfolded proteins, such as increased expression of heat shock proteins. The link with lipid metabolism is supported, for example, by the essential role of APOE in lipid transport in the brain (Kleiman, T., et al., 2006; Stone, D. J., et al., 2010). These processes have not been unequivocally ordered into a pathogenic cascade and the molecular mediators and correlates of each are largely unknown.

Microarray gene expression profiling provides an opportunity to observe processes that are common for normal aging, AD, and other neurodegenerative diseases, as well as to detect the differences between these conditions and disentangle their relationships. Towards that end, Applicants profiled post-mortem samples from non-demented and AD subjects and used gene co-expression network analysis to distinguish several major processes involved in brain aging and disease and to define the corresponding signature scores quantitatively. The invention herein is directed to biomarkers correlated to the underlying pathology, signature scores that can be used to monitor disease progression and to develop animal models for the study of disease pathology and the evaluation of therapeutics for the treatment of AD.

### SUMMARY OF THE INVENTION

In one aspect, the invention comprises four transcriptional biomarkers, BioAge (biological age), Alz (Alzheimer), Inflame (inflammation), and NdStress (neurodegenerative stress) that define gene expression variation in Alzheimer's disease (AD). BioAge captures the first principal component of variation and includes genes statistically associated with neuronal loss, glial activation, and lipid metabolism. BioAge typically increases with chronological age, but in AD it is prematurely expressed, as if, the subjects were 140 years old. A component of BioAge, Lipa, contains the AD risk factor APOE and reflects an apparent early disturbance in lipid metabolism. The rate of biological aging in AD patients, which was not explained by the BioAge, was instead associated with NdStress, which included genes related to protein folding and metabolism. Inflame, comprised of inflammatory cytokines and microglial genes, was broadly activated and appeared early in the disease process. In contrast, the disease specific Alz biomarker was selectively present only in the affected areas of the AD brain, appeared later in pathogenesis, and was enriched in genes associated with the signaling and cell adhesion changes during the epithelial to mesenchymal (EMT) transition.

In another aspect of the invention, the biomarkers can be used to calculate a biomarker score, or signature score, that can be used to diagnose Alzheimer's disease (AD) and monitor disease progression.

In still another aspect of the invention, the signature scores can be used to select animal models for the disease that can be used for the development and evaluation of therapeutics to treat Alzheimer's disease.

### BRIEF DESCRIPTION OF THE DRAWINGS

FIG. 1 is a representation of the heat map for the gene expression in PFC 1 (prefrontal cortex samples profiled in phase 1), which shows the hierarchical clustering of 4,000 of the most variable genes along x-axis. The subject samples are sorted along the y-axis (rows) according to the values of the first principal component of the complete dataset and labeled according to diagnosis (normal subjects in black, Alzheimer's disease (AD) subjects in red on the right).

FIGS. 2A and 2B are graphic representations of the aging score versus chronological age in PFC1. The box plots in FIG. 2A show the distribution of BioAge in different 5-year long age segments and the ANOVA p-values for the BioAge separation between normal and AD subjects in each chronological age segment. FIG. 2B shows the prediction of chronological age in an independent, normal cohort using BioAge. The postmortem prefrontal cortex samples from individuals of different age were profiled in an earlier study (GSE1572) (Lu, T. et al., 2009, Nature, 429:883-891). BioAge was calculated based on the average expression of several hundred genes from Tables 2 and 3.

FIGS. 3A and 3B are graphic representations of disease-specific metagenes. FIG. 3A shows a clustered gene-gene correlation matrix with strong mutual correlations between genes that were differentially expressed between AD and non-demented subjects from PFC1. FIG. 3B shows three outlined clusters corresponding to NdStress, Alz, and Inflame. The co-regulation of these genes is also shown in the bottom panel. Each line represents expression levels of individual genes in 55 PFC1 samples from non-demented and AD subjects sorted in the order of increasing BioAge. Only representative samples that scored in the top or bottom 3% for any of the biomarkers were selected for this figure to improve visualization.

FIG. 4 is a graphic representation of a plot matrix of mutual relationships between key aging and disease-specific biomarkers as well as chronological age. Each biomarker, Alz, NdStress, Inflame, Lipa, BioAge, is represented by its score in each sample based on the average gene expression of the contributing genes, listed in Tables 1-7. Non-demented PFC1 subjects are shown by black dots; AD subjects are shown by light gray dots. All pair-wise relationships between the biomarkers and with chronological age are shown.

FIGS. 5A-5B are graphic representations of the correlation of biomarker scores in PFC1 and VC1 (visual cortex samples profiled in phase 1) from the same individuals. Samples from non-demented and AD subjects are shown in black and light gray dots, respectively.

FIG. 6 is a graphic representation of the comparison of NdStress and Alz in AD and Huntington disease (HD) patients. AD subjects of PFC2 appear as black dots; HD subjects appear as light gray dots. The reference biomarker scores corresponding to non-demented individuals are represented by the dashed lines.

FIGS. 7A and 7B are schematic illustrations of a disease progression model. The trajectories of the biomarker BioAge change as a function of time (FIG. 7A), reflecting the relatively constant rate of aging in non-demented subjects (black), and the acceleration of the rate of aging in AD subjects (red). The dots at the end of the trajectory represent the postmortem state of the brain captured by the gene expression profiling. The state transition model (FIG. 7B) defines several broad categories for normal brains (N0-N3) and for diseased states (A1 and A2). The sequence of transitions and the associated gene expression biomarkers are shown by arrows.

FIGS. 8A-8C are graphic representations of the differential expression between AD and normal subjects of the PFC1 cohort. FIG. 8A shows the cumulative p-value distribution in a t-test, where the black line shows the number of sequences that can be detected for a given p-value cutoff, while the light gray line shows the level of false positives do to multiple testing. For example, at p<10E-6, about 18,000 genes can be detected. FIG. 8B is a Pareto diagram of variance explained by the first ten principal components. The first principal component dominates the distribution explaining 33% of the data variance. FIG. 8C is a comparison of the correlations between PC1 and individual genes in normal and AD subjects (see, FIG. 1).

FIG. 9 is a representation of a heat map showing the hierarchical clustering of seventeen selected genes involved with cell cycle regulation and DNA repair with the biomarker, BioAge. The role of these genes in the cell cycle and DNA repair is well established (Lu, T. et al., 2009, Nature, 429: 883-891). The subjects along the y-axis (rows) are sorted according to the values of the first principal component of the complete dataset and labeled according to diagnosis (normal subjects in black; AD subjects in light gray on the right) (see, FIG. 2).

FIG. 10 is a representation of a heat map showing the hierarchical clustering of the seventeen selected genes (FIG. 9) and their relationships with five biomarkers. The samples along the y-axis (rows) are sorted according to the values of the first principal component of the complete dataset and labeled according to diagnosis (normal samples in black, AD samples in light gray on the right). Only samples with a BioAge score of <0.4 are shown (see, FIG. 3).

FIGS. 11A-11D are graphic representations of the relationship of biomarker values between PFC1 and CR1 of the same individuals. Samples from non-demented and AD subjects are shown in black and light gray, respectively (see, FIG. 5).

FIGS. 12A-12D are graphic representations of the validation of the mutual relationships between key biomarkers in the PFC2 (prefrontal cortex samples profiled in phase 2) cohort, which contained non-demented (black), AD (light gray), and HD (dark gray) samples (see, FIG. 6).

FIG. 13 is a graphic representation of the human BioAge score projected into animal models. The box plots show the distribution of BioAge in week long age segments and the ANOVA p-values for the BioAge separation between wild-type (C57B) and an AD mouse model, NFEV (U.S. Pat. No. 7,432,414), in each chronological age segment. Two diets formulated by Test Diet (Richmond, Ind.) were used to feed the animals: normal and methionine-rich, that challenge metabolic pathways. The increased value of BioAge along the y-axis in the AD model with respect to the wild type animal demonstrated that the aging process in AD has progressed further than in wild type.

FIG. 14 is a graphic representation of the human Inflame score projected into an animal model. The box plots show the distribution of Inflame in week long age segments and the ANOVA p-values for the Inflame separation between wild-type (C57B) and an AD mouse model (NFEV) in each chronological age segment. Two diets were used to feed the animals: normal and methionine-rich, that challenge metabolic pathways. The increased value of Inflame along the y-axis in the AD model with respect to the wild type animal demonstrated that the inflammation process in AD was higher than in wild type.

FIG. 15 is a graphic representation of the NdStress biomarker in human blood. Blood samples from 7 control (CTRL), 8 AD-early, 10 AD (late), and 9 multiple sclerosis (MS) samples were profiled. The NdStress gene expression score was calculated after translating the biomarker gene symbols into human equivalents and matching the probes on the human microarray. The NdStress score shows elevated values in the subjects with neurodegenerative diseases in comparison to the control subjects. This suggests the possibility of using the NdStress biomarker as a peripheral diagnostic tool.

### DETAILED DESCRIPTION OF THE INVENTION

Microarray gene expression profiling provides an opportunity to observe the processes that are common for normal aging, Alzheimer's disease (AD), and other neurodegenerative diseases, as well as, to detect the differences between these conditions and disentangle their relationships. Applicants profiled several hundred post-mortem samples assembled in the Harvard Brain Tissue Resource Center (HBTRC, McLean Hospital, Belmont, Mass.) and used gene co-expression network analysis, Zhang, B. and Horvath, S., 2005, Stat. Appl. Genet. Mol. Biol., 4; Article 17; Tamayo, P. et al., 2007, Proc. Natl. Acad. Sci. USA, 104:5959-64; Carvalho, C. et al., 2008, J. Amer. Stat. Assn. 103:1438-1456; Oldham, M. C. et al., 2008, Nat. Neurosci., 11:1271-82; Miller, J. A., et al., 2008, J. Neurosci., 28:1410-20, to distinguish several major processes involved in brain aging and disease to qualitatively and quantitatively define a set of biomarkers and their corresponding signature scores. The correlation analysis of the signature scores between three profiled brain regions revealed systemic effects of the same disease processes on different brain regions. Applicants herein also provide a model of Alzheimer's disease progression that specifies the complex sequence of molecular pathological events associated with the disease. The inventive biomarkers and methods, i.e. signature scores, described herein can also be used to select animal models for the development and evaluation of therapeutics for the treatment of Alzheimer's disease (AD).

### DEFINITIONS

Unless defined otherwise, all technical and scientific terms used herein have the same meaning as commonly understood to one of ordinary skill in the art to which this invention belongs. The following definitions are provided in order to provide clarity with respect to terms as they are used in the specification and claims to describe various embodiments of the present invention.

As used herein, the term “Alzheimer's disease” or “AD” refers to any disease characterized by the accumulation of amyloid deposits in which the pathology results in some form of dementia or cognitive impairment. Amyloid deposits comprise a peptide, referred to as amyloid beta peptide, that aggregates to form an insoluble mass. Disease characterized by amyloid deposits include, but are not limited to Alzheimer's disease (AD), mild cognitive impairment, or other forms of memory loss or dementia.

As used herein, the term “normal” or “non-demented” refers to a subject who has not been previously diagnosed or who has not previously exhibited any clinical pathology related to Alzheimer's disease or any other form of cognitive impairment.

As used herein, the term “biomarker” refers to a list of genes known to be associated or correlated for which the gene expression in a particular tissue can be measured. The gene expression values for the correlated genes making up the biomarker can be used to calculate the signature score (Score) for the biomarker.

As used herein, the term “gene signature” or “signature score” or “Score” refers to a set of one or more differentially expressed genes that are statistically significant and characteristic of the biological differences between two or more cell samples, e.g., normal, non-demented and AD cells, cell samples from different cell types or tissue, or cells exposed to an agent or not. A signature may be expressed as a number of individual unique probes complementary to signature genes whose expression is detected when a cRNA product is used in microarray analysis or in a PCT reaction. A signature may be exemplified by a particular set of genes making up a biomarker. One means to calculate a signature or Score is provided in Example 4, in which the Score is equivalent to the average gene expression of the up-regulated genes minus the average gene expression for the down-regulated genes.

As used herein, the term “measuring expression levels,” or “obtaining expression level,” “detecting an expression level” and the like refers to methods that quantify a gene expression level of, for example, a transcript of a gene or a protein encoded by a gene, as well as methods that determine whether a gene or interest is expressed at all. Thus, an assay which provides a “yes” or “no” result without necessarily providing quantification of an amount of expression is an assay that “measures expression” as that term is used herein. Alternatively, a measured or obtained expression level may be expressed as any quantitative value, for example, a fold-change in expression, up or down, relative to a control gene or relative to the same gene in another sample, or a log ratio of expression, or any visual representation thereof, such as, for example a “heatmap” where a color intensity is representative of the amount of gene expression detected. Exemplary methods for detecting the level of expression of a gene include, but are not limited to, Northern blotting, dot or slot blots, reporter gene matrix (see, e.g., U.S. Pat. No. 5,569,588) nuclease protection, RT-PCR, microarray profiling, differential display, 2D gel electrophoresis, SELDI-TOF, ICAT, enzyme4 assay, antibody assay, and the like.

As used herein, the term “average gene expression” refers to arithmetic average of logarithm-transformed values of gene expression levels as measured on any applicable platform, as listed above.

As used herein, the term “classifier” refers to a property of a biomarker to distinguish groups of subjects and shown significant p-value in parametric (ANOVA) or non-parametric (Kruskal-Wallis) testing. For example, the classifier can be applied to samples collected from (1) the subject with AD and control subjects, (2) different neurodegenerative disease animal models As used herein, the term “sample” refers to a tissue specimen collected from human subjects or animal models As used herein, the term “subject” refers to an organism, such as a mammal, or to a cell sample, tissue sample or organ sample derived therefrom, including, for example, cultured cell lines, a biopsy, a blood sample, or a fluid sample containing a cell or a plurality of cells. In some instances, the subject or sample derived therefrom comprises a plurality of cell types. The organism may be an animal, including, but not limited to, an animal such as a mouse, rat, or dog, and is usually a mammal, such as a human.

### Biological Age

To identify gene expression changes corresponding to AD, we analyzed RNA specimens from more than 600 individuals with pathologically confirmed diagnoses of AD, Huntington's disease (HD), or age-matched controls (average post-mortem interval of 18 hours) using microarrays with over 40,000 unique probes. The brain regions profiled included dorsolateral prefrontal cortex (PFC), visual cortex (VC), and cerebellum (CR). These regions were chosen in part because, in AD, the PFC is impacted by the pathology while the latter two regions remain largely intact throughout most of the disease (Braak, H. and Braak, E., 1991, Acta. Neuropathol., 82: 239-259). The data were then analyzed by principal component analysis to assess the major patterns of gene expression variability. Genes that were highly correlated with the principal components were used to build signatures and biologically annotate the major sources of variance.

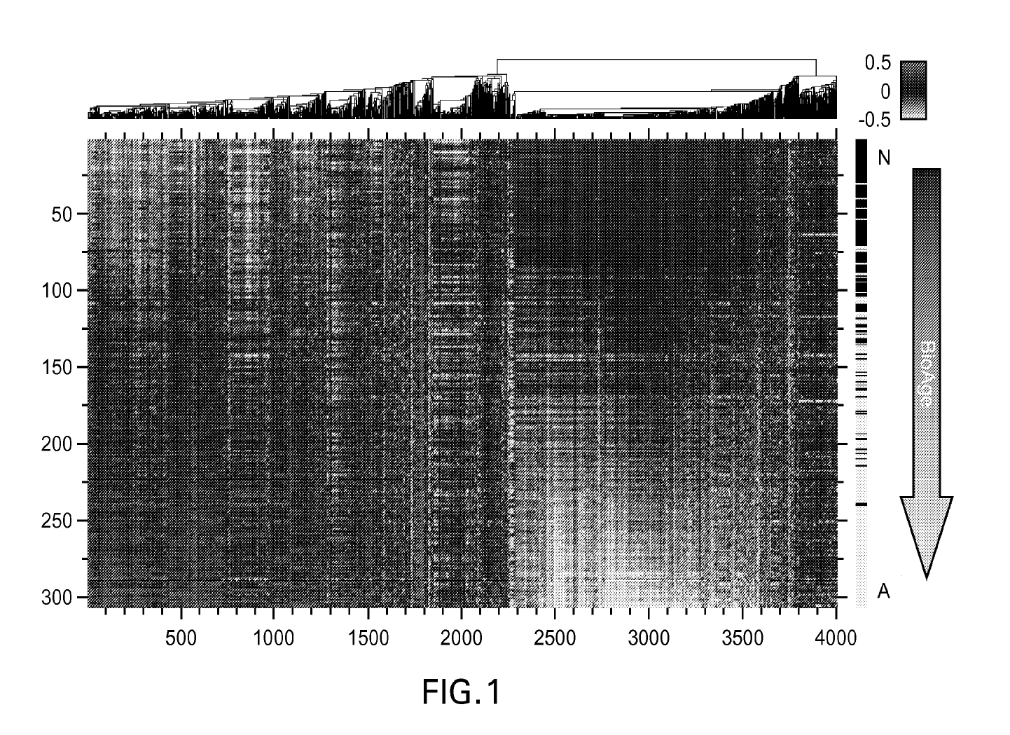

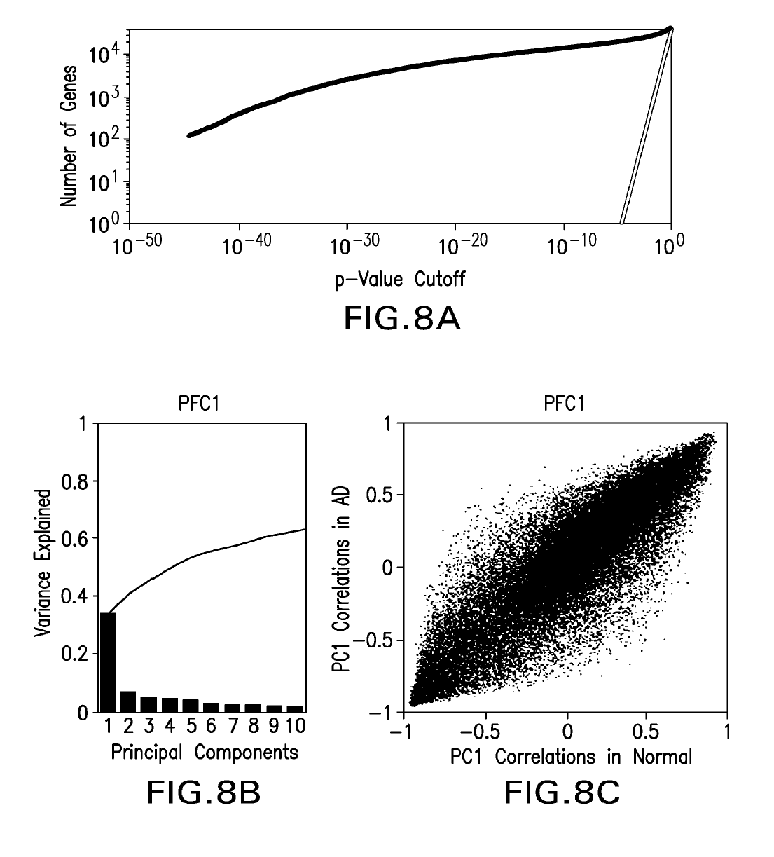

Analysis of differential gene expression in prefrontal cortex between non-demented individuals and AD patients revealed massive changes, with more than 18,000 transcripts significantly regulated (ANOVA p<10⁻⁶) by more than 28% (FIGS. 8A-8C). Much of this differential expression was due to a single gene expression pattern that defined the first principal component (PC1) in both AD and normal samples. PC1 explained 45% of the variance in the up-regulated genes and 60% of the variance in the down-regulated genes. As shown in the heat map in FIG. 1, AD and normal subjects dominated the opposite ends of this gene expression pattern, with some subjects from each group in the intermediate range. When normal and AD subjects were considered separately, it was largely the same genes that contributed to the PC1 pattern in both the AD and normal subjects, as shown by correlation analysis in FIGS. 8A-8C. This indicated that the same major biological process, as reflected in the gene expression, started in normal brains and continued developing in AD brains. Applicants found a significant correlation of PC1 with chronological age in non-demented individuals (ρ=0.58, p=9E-13), but not in AD patients (ρ=0.10, p=0.17), and concluded that this gene expression pattern captures normal aging processes in prefrontal cortex.

Tables 1-7 that follow show representative correlated genes that make up each biomarker and the average expression of which was used to calculate the biomarker score, i.e. the signature score. Tables 2 and 3 show the representative genes that were most up- (+BioAge) and down-regulated (-BioAge) with the biomarker, BioAge, and that were selected based on the strongest absolute correlations with PC 1.

TABLE 1
Correlated Genes for Lipa

| RefSeq Gene | | |
|---|---|---|
| Transcript | Gene | |
| Identification | Symbol | Gene Name/Description |
| ‘RSE_00000862609’ | ‘NOTCH2NL’ | ‘Notch homolog 2 (Drosophila) N-terminal like’ |
| ‘Contig56513_RC’ | ‘FIBIN’ | ‘fin bud initiation factor homolog (zebrafish)’ |
| ‘NM_013974’ | ‘DDAH2’ | ‘dimethylarginine dimethylaminohydrolase 2’ |
| ‘Contig52830_RC’ | ‘FAM59B’ | ‘family with sequence similarity 59, member B’ |
| ‘NM_018653’ | ‘GPRC5C’ | ‘G protein-coupled receptor, family C, group 5, member C’ |
| ‘Contig924_RC’ | ‘KIF1B’ | ‘kinesin family member 1B’ |
| ‘NM_018071’ | ‘KIF1B’ | ‘hypothetical protein FLJ10357’ |
| ‘Contig48473_RC’ | ‘C11orf93’ | ‘chromosome 11 open reading frame 93’ |
| ‘NM_003379’ | ‘EZR’ | ‘ezrin’ |
| ‘Contig41813_RC’ | ‘EZR’ | ‘hypothetical LOC645321’ |
| ‘Contig729_RC’ | ‘RIN2’ | ‘Ras and Rab interactor 2’ |
| ‘Contig53401_RC’ | ‘GLI3’ | ‘GLI family zinc finger 3’ |
| ‘Contig43791_RC’ | ‘TGFB2’ | ‘transforming growth factor, beta 2’ |
| ‘NM_016518’ | ‘PIPOX’ | ‘pipecolic acid oxidase’ |
| ‘NM_015642’ | ‘ZBTB20’ | ‘zinc finger and BTB domain containing 20’ |
| ‘Contig53742_RC’ | ‘STON2’ | ‘stonin 2’ |
| ‘NM_000029’ | ‘AGT’ | ‘angiotensinogen (serpin peptidase inhibitor, clade A, |
| | | member 8)’ |
| ‘NM_001400’ | ‘S1PR1’ | ‘sphingosine-1-phosphate receptor 1’ |
| ‘NM_006111’ | ‘ACAA2’ | ‘acetyl-Coenzyme A acyltransferase 2’ |
| ‘Contig52082_RC’ | ‘STK17B’ | ‘serine/threonine kinase 17b’ |
| ‘NM_000305’ | ‘PON2’ | ‘paraoxonase 2’ |
| ‘NM_001546’ | ‘ID4’ | ‘inhibitor of DNA binding 4, dominant negative helix-loop- |
| | | helix protein’ |
| ‘AL133574’ | ‘TEAD1’ | ‘TEA domain family member 1 (SV40 transcriptional |
| | | enhancer factor)’ |
| ‘NM_006984’ | ‘CLDN10’ | ‘claudin 10’ |
| ‘NM_004390’ | ‘CTSH’ | ‘cathepsin H’ |
| ‘Contig53719_RC’ | ‘C5orf33’ | ‘chromosome 5 open reading frame 33’ |
| ‘NM_000835’ | ‘GRIN2C’ | ‘glutamate receptor, ionotropic, N-methyl D-aspartate 2C’ |
| ‘Contig29647_RC’ | ‘LFNG’ | ‘LFNG O-fucosylpeptide 3-beta-N- |
| | | acetylglucosaminyltransferase’ |
| ‘NM_004905’ | ‘PRDX6’ | ‘peroxiredoxin 6’ |
| ‘NM_005954’ | ‘MT3’ | ‘metallothionein 3’ |
| ‘NM_000540’ | ‘RYR1’ | ‘ryanodine receptor 1 (skeletal)’ |
| ‘Contig58471_RC’ | ‘SLC27A1’ | ‘solute carrier family 27 (fatty acid transporter), member 1’ |
| ‘Contig41560_RC’ | ‘CPT1A’ | ‘carnitine palmitoyltransferase 1A (liver)’ |
| ‘NM_002775’ | ‘HTRA1’ | ‘HtrA serine peptidase 1’ |
| ‘AL049367’ | ‘GNG12’ | ‘guanine nucleotide binding protein (G protein), gamma 12’ |
| ‘NM_005086’ | ‘SSPN’ | ‘sarcospan (Kras oncogene-associated gene)’ |
| ‘NM_000137’ | ‘FAH’ | ‘fumarylacetoacetate hydrolase (fumarylacetoacetase)’ |
| ‘NM_002193’ | ‘INHBB’ | ‘inhibin, beta B’ |
| ‘NM_012190’ | ‘ALDH1L1’ | ‘aldehyde dehydrogenase 1 family, member L1’ |
| ‘NM_005031’ | ‘FXYD1’ | ‘FXYD domain containing ion transport regulator 1’ |
| ‘NM_001993’ | ‘F3’ | ‘coagulation factor III (thromboplastin, tissue factor)’ |
| ‘NM_003759’ | ‘SLC4A4’ | ‘solute carrier family 4, sodium bicarbonate cotransporter, |
| | | member 4’ |
| ‘AL049969’ | ‘PDLIM5’ | ‘PDZ and LIM domain 5’ |
| ‘NM_001492’ | ‘GDF1’ | ‘growth differentiation factor 1’ |
| ‘NM_001678’ | ‘ATP1B2’ | ‘ATPase, Na+/K+ transporting, beta 2 polypeptide’ |
| ‘Contig55727_RC’ | ‘SLC7A11’ | ‘solute carrier family 7, (cationic amino acid transporter, |
| | | y+ system) member 11’ |
| ‘Contig35000_RC’ | ‘SALL3’ | ‘sal-like 3 (Drosophila)’ |
| ‘NM_003986’ | ‘BBOX1’ | ‘butyrobetaine (gamma), 2-oxoglutarate dioxygenase |
| | | (gamma-butyrobetaine hydroxylase) 1’ |
| ‘NM_016246’ | ‘HSD17B14’ | ‘hydroxysteroid (17-beta) dehydrogenase 14’ |
| ‘AK002039’ | ‘MRVI1’ | ‘murine retrovirus integration site 1 homolog’ |
| ‘NM_006868’ | ‘RAB31’ | ‘RAB31, member RAS oncogene family’ |
| ‘AI076473_RC’ | ‘RUFY3’ | ‘RUN and FYVE domain containing 3’ |
| ‘NM_003672’ | ‘CDC14A’ | ‘CDC14 cell division cycle 14 homolog A (S. cerevisiae)’ |
| ‘NM_014738’ | ‘KIAA0195’ | ‘KIAA0195’ |
| ‘NM_000387’ | ‘SLC25A20’ | ‘solute carrier family 25 (carnitine/acylcarnitine |
| | | translocase), member 20’ |
| ‘NM_000041’ | ‘APOE’ | ‘apolipoprotein E’ |
| ‘NM_005274’ | ‘GNG5’ | ‘guanine nucleotide binding protein (G protein), gamma 5’ |
| ‘NM_005855’ | ‘RAMP1’ | ‘receptor (G protein-coupled) activity modifying protein 1’ |
| ‘NM_021082’ | ‘SLC15A2’ | ‘solute carrier family 15 (H+/peptide transporter), |
| | | member 2’ |
| ‘NM_000702’ | ‘ATP1A2’ | ‘ATPase, Na+/K+ transporting, alpha 2 (+) polypeptide’ |
| ‘NM_001182’ | ‘ALDH7A1’ | ‘aldehyde dehydrogenase 7 family, member A1’ |
| ‘AL080199’ | ‘ELOVL2’ | ‘elongation of very long chain fatty acids (FEN1/Elo2, |
| | | SUR4/Elo3, yeast)-like 2’ |
| ‘NM_000182’ | ‘HADHA’ | ‘hydroxyacyl-Coenzyme A dehydrogenase/3-ketoacyl- |
| | | Coenzyme A thiolase/enoyl-Coenzyme A hydratase |
| | | (trifunctional protein), alpha subunit’ |
| ‘NM_006227’ | ‘PLTP’ | ‘phospholipid transfer protein’ |
| ‘Contig37598’ | ‘ALDH6A1’ | ‘aldehyde dehydrogenase 6 family, member A1’ |
| ‘NM_000099’ | ‘CST3’ | ‘cystatin C’ |
| ‘Contig30480_RC’ | ‘BMPR1B’ | ‘bone morphogenetic protein receptor, type IB’ |
| ‘NM_000183’ | ‘HADHB” | ‘hydroxyacyl-Coenzyme A dehydrogenase/3-ketoacyl- |
| | | Coenzyme A thiolase/enoyl-Coenzyme A hydratase |
| | | (trifunctional protein), beta subunit’ |
| ‘NM_014817’ | ‘HADHB’ | ‘TLR4 interactor with leucine rich repeats’ |
| ‘NM_006271’ | ‘S100A1’ | ‘S100 calcium binding protein A1’ |
| ‘NM_006457’ | ‘PDLIM5’ | ‘PDZ and LIM domain 5’ |
| ‘Contig54726_RC’ | ‘USP3’ | ‘ubiquitin specific peptidase 3’ |
| ‘NM_016250’ | ‘NDRG2’ | ‘NDRG family member 2’ |
| ‘NM_006365’ | ‘C1orf61’ | ‘chromosome 1 open reading frame 61’ |
| ‘NM_005979’ | ‘S100A13’ | ‘S100 calcium binding protein A13’ |
| ‘NM_000690’ | ‘ALDH2’ | ‘aldehyde dehydrogenase 2 family (mitochondrial)’ |
| ‘NM_005245’ | ‘FAT1’ | ‘FAT tumor suppressor homolog 1 (Drosophila)’ |
| ‘NM_019025’ | ‘SMOX’ | ‘spermine oxidase’ |
| ‘NM_003362’ | ‘UNG’ | ‘uracil-DNA glycosylase’ |
| ‘NM_000280’ | ‘PAX6’ | ‘paired box 6’ |
| ‘NM_006719’ | ‘ABLIM1’ | ‘actin binding LIM protein 1’ |
| ‘NM_000676’ | ‘ADORA2B’ | ‘adenosine A2b receptor’ |
| ‘NM_004386’ | ‘NCAN’ | ‘neurocan’ |
| ‘NM_004466’ | ‘GPC5’ | ‘glypican 5’ |
| ‘NM_019886’ | ‘CHST7’ | ‘carbohydrate (N-acetylglucosamine 6-O) sulfo- |
| | | transferase 7’ |
| ‘NM_014214’ | ‘IMPA2’ | ‘inositol(myo)-1(or 4)-monophosphatase 2’ |
| ‘NM_001979’ | ‘EPHX2’ | ‘epoxide hydrolase 2, cytoplasmic’ |
| ‘NM_003098’ | ‘SNTA1’ | ‘syntrophin, alpha 1 (dystrophin-associated protein A1, |
| | | 59 kDa, acidic component)’ |
| ‘AB011540’ | ‘LRP4’ | ‘low density lipoprotein receptor-related protein 4’ |
| ‘AB037778’ | ‘NHSL1’ | ‘NHS-like 1’ |
| ‘NM_002637’ | ‘PHKA1’ | ‘phosphorylase kinase, alpha 1 (muscle)’ |
| ‘Contig1667_RC’ | ‘SSPN’ | ‘sarcospan (Kras oncogene-associated gene)’ |
| ‘AB037858’ | ‘LRRC8A’ | ‘leucine rich repeat containing 8 family, member A’ |
| ‘NM_006623’ | ‘PHGDH’ | ‘phosphoglycerate dehydrogenase’ |
| ‘NM_000168’ | ‘GLI3’ | ‘GLI family zinc finger 3’ |
| ‘NM_018281’ | ‘ECHDC2’ | ‘enoyl Coenzyme A hydratase domain containing 2’ |
| ‘M37712’ | ‘GPR125’ | ‘G protein-coupled receptor 125’ |
| ‘NM_000362’ | ‘TIMP3’ | ‘TIMP metallopeptidase inhibitor 3’ |
| ‘Contig55022_RC’ | ‘ASRGL1’ | ‘asparaginase like 1’ |
| ‘NM_002313’ | ‘ABLIM1’ | ‘actin binding LIM protein 1’ |
| ‘NM_000120’ | ‘EPHX1’ | ‘epoxide hydrolase 1, microsomal (xenobiotic)’ |
| ‘NM_003272’ | ‘GPR137B’ | ‘G protein-coupled receptor 137B’ |
| ‘NM_001899’ | ‘CST4’ | ‘cystatin S’ |
| ‘NM_000381’ | ‘MID1’ | ‘midline 1 (Opitz/BBB syndrome)’ |
| ‘NM_002206’ | ‘ITGA7’ | ‘integrin, alpha 7’ |
| ‘AL137578’ | ‘EMX2OS’ | ‘EMX2 opposite strand (non-protein coding)’ |
| ‘Contig57903_RC’ | ‘SASH1’ | ‘SAM and SH3 domain containing 1’ |
| ‘NM_014799’ | ‘HEPH’ | ‘hephaestin’ |
| ‘Contig45964_RC’ | ‘NTRK2’ | ‘neurotrophic tyrosine kinase, receptor, type 2’ |
| ‘NM_003713’ | ‘PPAP2B’ | ‘phosphatidic acid phosphatase type 2B’ |
| ‘NM_016938’ | ‘EFEMP2’ | ‘EGF-containing fibulin-like extracellular matrix protein 2’ |
| ‘NM_020659’ | ‘TTYH1’ | ‘tweety homolog 1 (Drosophila)’ |
| ‘NM_004393’ | ‘DAG1’ | ‘dystroglycan 1 (dystrophin-associated glycoprotein 1)’ |
| ‘NM_017640’ | ‘LRRC16A’ | ‘leucine rich repeat containing 16A’ |
| ‘NM_000115’ | ‘EDNRB’ | ‘endothelin receptor type B’ |
| ‘NM_017577’ | ‘GRAMD1C’ | ‘GRAM domain containing 1C’ |
| ‘NM_014745’ | ‘FAM38A’ | ‘family with sequence similarity 38, member A’ |
| ‘Contig48971_RC’ | ‘CHDH’ | ‘choline dehydrogenase’ |
| ‘Contig3124_RC’ | ‘PSMB7’ | ‘proteasome (prosome, macropain) subunit, beta type, 7’ |
| ‘NM_007117’ | ‘FAM107A’ | ‘family with sequence similarity 107, member A’ |
| ‘AL137567’ | ‘RIMKLB’ | ‘ribosomal modification protein rimK-like family |
| | | member B’ |
| ‘NM_006783’ | ‘GJB6’ | ‘gap junction protein, beta 6, 30 kDa’ |
| ‘NM_004171’ | ‘SLC1A2’ | ‘solute carrier family 1 (glial high affinity glutamate |
| | | transporter), member 2’ |
| ‘NM_004172’ | ‘SLC1A3’ | ‘solute carrier family 1 (glial high affinity glutamate |
| | | transporter), member 3’ |
| ‘AL157452’ | ‘SLC1A2’ | ‘solute carrier family 1 (glial high affinity glutamate |
| | | transporter), member 2’ |
| ‘NM_000165’ | ‘GJA1’ | ‘gap junction protein, alpha 1, 43 kDa’ |
| ‘NM_001036’ | ‘RYR3’ | ‘ryanodine receptor 3’ |
| ‘Contig54761_RC’ | ‘CAMTA1’ | ‘calmodulin binding transcription activator 1’ |
| ‘AF131748’ | ‘SUCLG2’ | ‘succinate-CoA ligase, GDP-forming, beta subunit’ |
| ‘Contig44111_RC’ | ‘PHKA1’ | ‘phosphorylase kinase, alpha 1 (muscle)’ |
| ‘Contig56689_RC’ | ‘POU2F1’ | ‘POU class 2 homeobox 1’ |
| ‘AI393246_RC’ | ‘CD2AP’ | ‘CD2-associated protein’ |
| ‘NM_003500’ | ‘ACOX2’ | ‘acyl-Coenzyme A oxidase 2, branched chain’ |
| ‘NM_004252’ | ‘SLC9A3R1’ | ‘solute carrier family 9 (sodium/hydrogen exchanger), |
| | | member 3 regulator 1’ |
| ‘Contig27908_RC’ | ‘NPAS3’ | ‘neuronal PAS domain protein 3’ |
| ‘NM_002999’ | ‘SDC4’ | ‘syndecan 4’ |
| ‘NM_017435’ | ‘SLCO1C1’ | ‘solute carrier organic anion transporter family, |
| | | member 1C1’ |
| ‘Contig63683_RC’ | ‘EPB41L5’ | ‘erythrocyte membrane protein band 4.1 like 5’ |
| ‘NM_133443’ | ‘GPT2’ | ‘glutamic pyruvate transaminase (alanine amino- |
| | | transferase) 2’ |
| ‘Contig693_RC’ | ‘NFIA’ | ‘nuclear factor I/A’ |
| ‘NM_130468’ | ‘CHST14’ | ‘carbohydrate (N-acetylgalactosamine 4-0) sulfo- |
| | | transferase 14’ |
| ‘NM_052831’ | ‘C6orf192’ | ‘chromosome 6 open reading frame 192’ |
| ‘NM_031313’ | ‘ALPPL2’ | ‘alkaline phosphatase, placental-like 2’ |
| ‘NM_024843’ | ‘CYBRD1’ | ‘cytochrome b reductase 1’ |
| ‘AK055239’ | ‘ARSD’ | ‘arylsulfatase D’ |
| ‘NM_015162’ | ‘ACSBG1’ | ‘acyl-CoA synthetase bubblegum family member 1’ |
| ‘NM_024071’ | ‘ZFYVE21’ | ‘zinc finger, FYVE domain containing 21’ |
| ‘NM_024723’ | ‘MICALL2’ | ‘MICAL-like 2’ |
| ‘AK055553’ | ‘TTC28’ | ‘tetratricopeptide repeat domain 28’ |
| ‘NM_138463’ | ‘TLCD1’ | ‘TLC domain containing 1’ |
| ‘NM_032644’ | ‘PPARA’ | ‘peroxisome proliferator-activated receptor alpha’ |
| ‘NM_080388’ | ‘S100A16’ | ‘S100 calcium binding protein A16’ |
| ‘AL359558’ | ‘MCC’ | ‘mutated in colorectal cancers’ |
| ‘NM_024042’ | ‘METRN’ | ‘meteorin, glial cell differentiation regulator’ |
| ‘AK056229’ | ‘METRN” | ‘hypothetical protein LOC727973’ |
| ‘NM_025080’ | ‘ASRGL1’ | ‘asparaginase like 1’ |
| ‘AK024775’ | ‘DPY19L3’ | ‘dpy-19-like 3 (C. elegans)’ |
| ‘NM_021923’ | ‘FGFRL1’ | ‘fibroblast growth factor receptor-like 1’ |
| ‘NM_052953’ | ‘LRRC3B’ | ‘leucine rich repeat containing 3B’ |
| ‘NM_014562’ | ‘OTX1’ | ‘orthodenticle homeobox 1’ |
| ‘AK026728’ | ‘AQP4’ | ‘aquaporin 4’ |
| ‘NM_005647’ | ‘TBL1X’ | ‘transducin (beta)-like 1X-linked’ |
| ‘ENST00000295535’ | ‘ATP13A4’ | ‘ATPase type 13A4’ |
| ‘Contig4539’ | ‘RHOBTB3’ | ‘Rho-related BTB domain containing 3’ |
| ‘NM_020663’ | ‘RHOJ’ | ‘ras homolog gene family, member J’ |
| ‘NM_032491’ | ‘RFX4’ | ‘regulatory factor X, 4 (influences HLA class II |
| | | expression)’ |
| ‘NM_138284’ | ‘IL17D’ | ‘interleukin 17D’ |
| ‘NM_031279’ | ‘AGXT2L1’ | ‘alanine-glyoxylate aminotransferase 2-like 1’ |
| ‘NM_024952’ | ‘C14orf159’ | ‘chromosome 14 open reading frame 159’ |
| ‘NM_032173’ | ‘ZNRF3’ | ‘zinc and ring finger 3’ |
| ‘NM_004098’ | ‘EMX2’ | ‘empty spiracles homeobox 2’ |
| ‘NM_031481’ | ‘SLC25A18’ | ‘solute carrier family 25 (mitochondrial carrier), |
| | | member 18’ |
| ‘NM_024728’ | ‘C7orf10’ | ‘chromosome 7 open reading frame 10’ |
| ‘NM_032289’ | ‘PSD2’ | ‘pleckstrin and Sec7 domain containing 2’ |
| ‘AK027101’ | ‘PPARA’ | ‘peroxisome proliferator-activated receptor alpha’ |
| ‘NM_024911’ | ‘GPR177’ | ‘G protein-coupled receptor 177’ |
| ‘NM_003302’ | ‘TRIP6’ | ‘thyroid hormone receptor interactor 6’ |
| ‘NM_175622’ | ‘MT1JP’ | ‘metallothionein 1J (pseudogene)’ |
| ‘NM_033044’ | ‘MACF1’ | ‘microtubule-actin crosslinking factor 1’ |
| ‘NM_003944’ | ‘SELENBP1’ | ‘selenium binding protein 1’ |
| ‘NM_014033’ | ‘METTL7A’ | ‘methyltransferase like 7A’ |
| ‘NM_015035’ | ‘ZHX3’ | ‘zinc fingers and homeoboxes 3’ |
| ‘NM_032092’ | ‘PCDHGA11’ | ‘protocadherin gamma subfamily A, 11’ |
| ‘NM_000142’ | ‘FGFR3’ | ‘fibroblast growth factor receptor 3’ |
| ‘NM_001719’ | ‘BMP7’ | ‘bone morphogenetic protein 7’ |
| ‘NM_005682’ | ‘GPR56’ | ‘G protein-coupled receptor 56’ |
| ‘NM_152459’ | ‘C16orf89’ | ‘chromosome 16 open reading frame 89’ |
| ‘NM_012304’ | ‘FBXL7’ | ‘F-box and leucine-rich repeat protein 7’ |
| ‘NM_000391’ | ‘TPP1’ | ‘tripeptidyl peptidase I’ |
| ‘NM_004767’ | ‘GPR37L1’ | ‘G protein-coupled receptor 37 like 1’ |
| ‘NM_004840’ | ‘ARHGEF6’ | ‘Rac/Cdc42 guanine nucleotide exchange factor (GEF) 6’ |
| ‘NM_018912’ | ‘PCDHGA1’ | ‘protocadherin gamma subfamily A, 1’ |
| ‘NM_021913’ | ‘AXL’ | ‘AXL receptor tyrosine kinase’ |
| ‘NM_032192’ | ‘PPP1R1B’ | ‘protein phosphatase 1, regulatory (inhibitor) subunit 1B’ |
| ‘NM_006108’ | ‘SPON1’ | ‘spondin 1, extracellular matrix protein’ |
| ‘NM_015541’ | ‘LRIG1’ | ‘leucine-rich repeats and immunoglobulin-like domains 1’ |
| ‘NM_174933’ | ‘PHYHD1’ | ‘phytanoyl-CoA dioxygenase domain containing 1’ |
| ‘NM_080911’ | ‘UNG’ | ‘uracil-DNA glycosylase’ |
| ‘NM_172110’ | ‘EYA2’ | ‘eyes absent homolog 2 (Drosophila)’ |
| ‘NM_005559’ | ‘LAMA1’ | ‘laminin, alpha 1’ |
| ‘NM_018920’ | ‘PCDHGA7’ | ‘protocadherin gamma subfamily A, 7’ |
| ‘NM_005271’ | ‘GLUD1’ | ‘glutamate dehydrogenase 1’ |
| ‘NM_182848’ | ‘CLDN10’ | ‘claudin 10’ |
| ‘NM_023927’ | ‘GRAMD3’ | ‘GRAM domain containing 3’ |
| ‘NM_000346’ | ‘SOX9’ | ‘SRY (sex determining region Y)-box 9’ |
| ‘NM_032119’ | ‘GPR98’ | ‘G protein-coupled receptor 98’ |
| ‘NM_003217’ | ‘TMBIM6’ | ‘transmembrane BAX inhibitor motif containing 6’ |
| ‘NM_172087’ | ‘TNFSF13’ | ‘tumor necrosis factor (ligand) superfamily, member 13’ |
| ‘NM_032088’ | ‘PCDHGA8’ | ‘protocadherin gamma subfamily A, 8’ |
| ‘NM_003848’ | ‘SUCLG2’ | ‘succinate-CoA ligase, GDP-forming, beta subunit’ |
| ‘NM_015430’ | ‘PAMR1’ | ‘peptidase domain containing associated with muscle |
| | | regeneration 1’ |
| ‘NM_030906’ | ‘STK33’ | ‘serine/threonine kinase 33’ |
| ‘NM_032466’ | ‘ASPH’ | ‘aspartate beta-hydroxylase’ |
| ‘NM_003038’ | ‘SLC1A4’ | ‘solute carrier family 1 (glutamate/neutral amino acid |
| | | transporter), member 4’ |
| ‘NM_002998’ | ‘SDC2’ | ‘syndecan 2’ |
| ‘NM_144579’ | ‘SFXN5’ | ‘sideroflexin 5’ |
| ‘NM_015278’ | ‘SASH1’ | ‘SAM and SH3 domain containing 1’ |
| ‘NM_018913’ | ‘PCDHGA10’ | ‘protocadherin gamma subfamily A, 10’ |
| ‘NM_005589’ | ‘ALDH6A1’ | ‘aldehyde dehydrogenase 6 family, member A1’ |
| ‘NM_005426’ | ‘TP53BP2’ | ‘tumor protein p53 binding protein, 2’ |
| ‘NM_005524’ | ‘HES1’ | ‘hairy and enhancer of split 1, (Drosophila)’ |
| ‘NM_030935’ | ‘TSC22D4’ | ‘TSC22 domain family, member 4’ |
| ‘NM_015069’ | ‘ZNF423’ | ‘zinc finger protein 423’ |
| ‘NM_000940’ | ‘PON3’ | ‘paraoxonase 3’ |
| ‘NM_177414’ | ‘PPAP2B’ | ‘phosphatidic acid phosphatase type 2B’ |
| ‘NM_020925’ | ‘CACHD1’ | ‘cache domain containing 1’ |
| ‘NM_153362’ | ‘PRSS35’ | ‘protease, serine, 35’ |
| ‘NM_170782’ | ‘KCNN3’ | ‘potassium intermediate/small conductance calcium- |
| | | activated channel, subfamily N, member 3’ |
| ‘NM_003735’ | ‘PCDHGA12’ | ‘protocadherin gamma subfamily A, 12’ |
| ‘NM_053279’ | ‘FAM167A’ | ‘family with sequence similarity 167, member A’ |
| ‘NM_014079’ | ‘KLF15’ | ‘Kruppel-like factor 15’ |
| ‘NM_021939’ | ‘FKBP10’ | ‘FK506 binding protein 10, 65 kDa’ |
| ‘NM_003736’ | ‘PCDHGB4’ | ‘protocadherin gamma subfamily B, 4’ |
| ‘NM_152444’ | ‘PTGR2’ | ‘prostaglandin reductase 2’ |
| ‘NM_152288’ | ‘ORAI3’ | ‘ORAI calcium release-activated calcium modulator 3’ |
| ‘NM_012344’ | ‘NTSR2’ | ‘neurotensin receptor 2’ |
| ‘NM_016499’ | ‘TMEM216’ | ‘transmembrane protein 216’ |
| ‘NM_018925’ | ‘PCDHGB5’ | ‘protocadherin gamma subfamily B, 5’ |
| ‘NM_017711’ | ‘GDPD2’ | ‘glycerophosphodiester phosphodiesterase domain |
| | | containing 2’ |
| ‘NM_005595’ | ‘NFIA’ | ‘nuclear factor I/A’ |
| ‘NM_003732’ | ‘EIF4EBP3’ | ‘eukaryotic translation initiation factor 4E binding |
| | | protein 3’ |
| ‘NM_175617’ | ‘MT1E’ | ‘metallothionein 1E’ |
| ‘NM_018929’ | ‘PCDHGC5’ | ‘protocadherin gamma subfamily C, 5’ |
| ‘NM_000273’ | ‘GPR143’ | ‘G protein-coupled receptor 143’ |
| ‘NM_175885’ | ‘FAM181B’ | ‘family with sequence similarity 181, member B’ |
| ‘NM_018924’ | ‘PCDHGB3’ | ‘protocadherin gamma subfamily B, 3’ |
| ‘NM_138737’ | ‘HEPH’ | ‘hephaestin’ |
| ‘NM_018921’ | ‘PCDHGA9’ | ‘protocadherin gamma subfamily A, 9’ |
| ‘NM_018916’ | ‘PCDHGA3’ | ‘protocadherin gamma subfamily A, 3’ |
| ‘NM_001604’ | ‘PAX6’ | ‘paired box 6’ |
| ‘NM_018171’ | ‘APPL2’ | ‘adaptor protein, phosphotyrosine interaction, PH domain |
| | | and leucine zipper containing 2’ |
| ‘NM_031442’ | ‘TMEM47’ | ‘transmembrane protein 47’ |
| ‘NM_003702’ | ‘RGS20’ | ‘regulator of G-protein signaling 20’ |
| ‘NM_004096’ | ‘EIF4EBP2’ | ‘eukaryotic translation initiation factor 4E binding |
| | | protein 2’ |
| ‘NM_134433’ | ‘RFX2’ | ‘regulatory factor X, 2 (influences HLA class II |
| | | expression)’ |
| ‘NM_058179’ | ‘PSAT1’ | ‘phosphoserine aminotransferase 1’ |
| ‘NM_015645’ | ‘C1QTNF5’ | ‘C1q and tumor necrosis factor related protein 5’ |
| ‘NM_173638’ | ‘NBPF15’ | ‘neuroblastoma breakpoint family, member 15’ |
| ‘NM_018915’ | ‘PCDHGA2’ | ‘protocadherin gamma subfamily A, 2’ |
| ‘NM_012121’ | ‘CDC42EP4’ | ‘CDC42 effector protein (Rho GTPase binding) 4’ |
| ‘NM_139202’ | ‘MLC1’ | ‘megalencephalic leukoencephalopathy with subcortical |
| | | cysts 1’ |
| ‘NM_020428’ | ‘SLC44A2’ | ‘solute carrier family 44, member 2’ |
| ‘NM_018922’ | ‘PCDHGB1’ | ‘protocadherin gamma subfamily B, 1’ |
| ‘NM_021943’ | ‘ZFAND3’ | ‘zinc finger, AN1-type domain 3’ |
| ‘NM_018919’ | ‘PCDHGA6’ | ‘protocadherin gamma subfamily A, 6’ |
| ‘NM_018927’ | ‘PCDHGB7’ | ‘protocadherin gamma subfamily B, 7’ |
| ‘NM_002825’ | ‘PTN’ | ‘pleiotrophin’ |
| ‘NM_018928’ | ‘PCDHGC4’ | ‘protocadherin gamma subfamily C, 4’ |
| ‘NM_031934’ | ‘RAB34’ | ‘RAB34, member RAS oncogene family’ |
| ‘NM_005228’ | ‘EGFR’ | ‘epidermal growth factor receptor (erythroblastic leukemia |
| | | viral (v-erb-b) oncogene homolog, avian)’ |
| ‘NM_018397’ | ‘CHDH’ | ‘choline dehydrogenase’ |
| ‘NM_016081’ | ‘PALLD’ | ‘palladin, cytoskeletal associated protein’ |
| ‘NM_153000’ | ‘APCDD1’ | ‘adenomatosis polyposis coli down-regulated 1’ |
| ‘NM_015595’ | ‘APCDD1” | ‘Src homology 3 domain-containing guanine nucleotide |
| | | exchange factor’ |
| ‘NM_153342’ | ‘TMEM150A’ | ‘transmembrane protein 150A’ |
| ‘NM_024766’ | ‘C2orf34’ | ‘chromosome 2 open reading frame 34’ |
| ‘NM_152661’ | ‘C2orf34” | ‘hypothetical LOC440556’ |
| ‘NM_138375’ | ‘CABLES1’ | ‘Cdk5 and Abl enzyme substrate 1’ |
| ‘NM_024408’ | ‘NOTCH2’ | ‘Notch homolog 2 (Drosophila)’ |
| ‘NM_012334’ | ‘MYO10’ | ‘myosin X’ |
| ‘NM_003106’ | ‘SOX2’ | ‘SRY (sex determining region Y)-box 2’ |
| ‘NM_152725’ | ‘SLC39A12’ | ‘solute carrier family 39 (zinc transporter), member 12’ |
| ‘NM_018923’ | ‘PCDHGB2’ | ‘protocadherin gamma subfamily B, 2’ |
| ‘NM_018918’ | ‘PCDHGA5’ | ‘protocadherin gamma subfamily A, 5’ |
| ‘NM_018917’ | ‘PCDHGA4’ | ‘protocadherin gamma subfamily A, 4’ |
| ‘NM_170721’ | ‘MSI2’ | ‘musashi homolog 2 (Drosophila)’ |
| ‘NM_020524’ | ‘PBXIP1’ | ‘pre-B-cell leukemia homeobox interacting protein 1’ |
| ‘NM_144672’ | ‘OTOA’ | ‘otoancorin’ |
| ‘NM_152737’ | ‘RNF182’ | ‘ring finger protein 182’ |
| ‘NM_012417’ | ‘PITPNC1’ | ‘phosphatidylinositol transfer protein, cytoplasmic 1’ |
| ‘NM_170726’ | ‘ALDH4A1’ | ‘aldehyde dehydrogenase 4 family, member A1’ |
| ‘NM_025201’ | ‘PLEKHO2’ | ‘pleckstrin homology domain containing, family O |
| | | member 2’ |
| ‘NM_021948’ | ‘BCAN’ | ‘brevican’ |
| ‘NM_032501’ | ‘ACSS1’ | ‘acyl-CoA synthetase short-chain family member 1’ |
| ‘NM_025149’ | ‘ACSF2’ | ‘acyl-CoA synthetase family member 2’ |
| ‘NM_005631’ | ‘SMO’ | ‘smoothened homolog (Drosophila)’ |
| ‘NM_033103’ | ‘RHPN2’ | ‘rhophilin, Rho GTPase binding protein 2’ |
| ‘NM_004099’ | ‘STOM’ | ‘stomatin’ |
| ‘NM_173462’ | ‘PAPLN’ | ‘papilin, proteoglycan-like sulfated glycoprotein’ |
| ‘NM_033290’ | ‘MID1’ | ‘midline 1 (Opitz/BBB syndrome)’ |
| ‘NM_002394’ | ‘SLC3A2’ | ‘solute carrier family 3 (activators of dibasic and neutral |
| | | amino acid transport), member 2’ |
| ‘NM_005952’ | ‘MT1X’ | ‘metallothionein 1X’ |
| ‘NM_018926’ | ‘PCDHGB6’ | ‘protocadherin gamma subfamily B, 6’ |
| ‘NM_178507’ | ‘OAF’ | ‘OAF homolog (Drosophila)’ |
| ‘NM_000696’ | ‘ALDH9A1’ | ‘aldehyde dehydrogenase 9 family, member A1’ |
| ‘NM_032208’ | ‘ANTXR1’ | ‘anthrax toxin receptor 1’ |
| ‘NM_176870’ | ‘MT1M’ | ‘metallothionein 1M’ |
| ‘NM_003269’ | ‘NR2E1’ | ‘nuclear receptor subfamily 2, group E, member 1’ |
| ‘NM_000503’ | ‘EYA1’ | ‘eyes absent homolog 1 (Drosophila)’ |
| ‘NM_006832’ | ‘FERMT2’ | ‘fermitin family homolog 2 (Drosophila)’ |
| ‘NM_021902’ | ‘FXYD1’ | ‘FXYD domain containing ion transport regulator 1’ |
| ‘NM_175875’ | ‘SIX5’ | ‘SDC homeobox 5’ |
| ‘NM_138415’ | ‘PHF21B’ | ‘PHD finger protein 21B’ |
| ‘BC040156’ | ‘PHF21B” | ‘hypothetical protein LOC284570’ |
| ‘AK092579’ | ‘IL17RD’ | ‘interleukin 17 receptor D’ |
| ‘BC040678’ | ‘IL17RD” | ‘hypothetical LOC643763’ |
| ‘HSS00130473’ | ‘IL17RD’ | ‘similar to hCG2038817’ |
| ‘hCT1644663.3’ | ‘ATP13A5’ | ‘ATPase type 13A5’ |
| ‘AL832622’ | ‘NBPF11’ | ‘neuroblastoma breakpoint family, member 11’ |
| ‘AL365371’ | ‘FKBP10’ | ‘FK506 binding protein 10, 65 kDa’ |
| ‘hCT1970462’ | ‘ACSF2’ | ‘acyl-CoA synthetase family member 2’ |
| ‘AB033041’ | ‘VANGL2’ | ‘vang-like 2 (van gogh, Drosophila)’ |
| ‘AL357198’ | ‘TP53BP2’ | ‘tumor protein p53 binding protein, 2’ |
| ‘NM_004635’ | ‘MAPKAPK3’ | ‘mitogen-activated protein kinase-activated protein |
| | | kinase 3’ |
| ‘NM_002588’ | ‘PCDHGC3’ | ‘protocadherin gamma subfamily C, 3’ |
| ‘NM_002213’ | ‘ITGB5’ | ‘integrin, beta 5’ |
| ‘NM_017901.2’ | ‘TPCN1’ | ‘two pore segment channel 1’ |
| ‘NM_080757’ | ‘MT1P3’ | ‘metallothionein 1 pseudogene 3’ |
| ‘NM_172089’ | ‘TNFSF12- | ‘TNFSF12-TNFSF13 readthrough’ |
| | TNFSF13’ | |
| ‘ENST00000264245’ | ‘ARHGAP31’ | ‘Rho GTPase activating protein 31’ |
| ‘NM_003269’ | ‘NR2E1’ | ‘nuclear receptor subfamily 2, group E, member 1’ |
| ‘NM_005036’ | ‘PPARA’ | ‘peroxisome proliferator-activated receptor alpha’ |
| ‘NM_005502’ | ‘ABCA1’ | ‘ATP-binding cassette, sub-family A (ABC1), member 1’ |
| ‘NM_003038’ | ‘SLC1A4’ | ‘solute carrier family 1 (glutamate/neutral amino acid |
| | | transporter), member 4’ |
| ‘NM_000387’ | ‘SLC25A20’ | ‘solute carrier family 25 (carnitine/acylcarnitine |
| | | translocase), member 20’ |
| ‘NM_004252’ | ‘SLC9A3R1’ | ‘solute carrier family 9 (sodium/hydrogen exchanger), |
| | | member 3 regulator 1’ |
| ‘NM_001979’ | ‘EPHX2’ | ‘epoxide hydrolase 2, cytoplasmic’ |
| ‘NM_004172’ | ‘SLC1A3’ | ‘solute carrier family 1 (glial high affinity glutamate |
| | | transporter), member 3’ |
| ‘NM_031481’ | ‘SLC25A18’ | ‘solute carrier family 25 (mitochondrial carrier), |
| | | member 18’ |
| ‘NM_000029_sat’ | ‘AGT’ | ‘angiotensinogen (serpin peptidase inhibitor, clade A, |
| | | member 8)’ |
| ‘NM_021082’ | ‘SLC15A2’ | ‘solute carrier family 15 (H+/peptide transporter), |
| | | member 2’ |
| ‘NM_000041_sat’ | ‘APOE’ | ‘apolipoprotein E’ |
| ‘NM_000120’ | ‘EPHX1’ | ‘epoxide hydrolase 1, microsomal (xenobiotic)’ |
| ‘NM_005072’ | ‘SLC12A4’ | ‘solute carrier family 12 (potassium/chloride transporters), |
| | | member 4’ |
| ‘NM_017435’ | ‘SLCO1C1’ | ‘solute carrier organic anion transporter family, |
| | | member 1C1’ |
| ‘NM_005951’ | ‘MT1H’ | ‘metallothionein 1H’ |
| ‘AY369853’ | ‘MT1H’ | ‘solute carrier family 1 (glial high affinity glutamate |
| | | transporter), member 2’ |
| ‘NM_000240’ | ‘MAOA’ | ‘monoamine oxidase A’ |
| ‘NM_003759’ | ‘SLC4A4’ | ‘solute carrier family 4, sodium bicarbonate cotransporter, |
| | | member 4’ |
| ‘NM_172087’ | ‘TNFSF13’ | ‘tumor necrosis factor (ligand) superfamily, member 13’ |
| ‘NM_004171’ | ‘SLC1A2’ | ‘solute carrier family 1 (glial high affinity glutamate |
| | | transporter), member 2’ |
| ‘NM_031279’ | ‘AGXT2L1’ | ‘alanine-glyoxylate aminotransferase 2-like 1’ |
| ‘NM_024728’ | ‘C7orf10’ | ‘chromosome 7 open reading frame 10’ |
| ‘NM_005954’ | ‘MT3’ | ‘metallothionein 3’ |

TABLE 2
Correlated Genes for +BioAge

| RefSeq Gene | | |
|---|---|---|
| Transcript | Gene | |
| Identification | Symbol | Gene Name/Description |
| ‘NM_006790’ | ‘MYOT’ | ‘myotilin’ |
| ‘NM_001085’ | ‘SERPINA3’ | ‘serpin peptidase inhibitor, clade A (alpha-1 |
| | | antiproteinase, antitrypsin), member 3’ |
| ‘NM_01747’ | ‘CAPG’ | ‘capping protein (actin filament), gelsolin-like’ |
| ‘AL117477’ | ‘PHF19’ | ‘PHD finger protein 19’ |
| ‘NM_005730’ | ‘CTDSP2’ | ‘CTD (carboxy-terminal domain, RNA polymerase II, |
| | | polypeptide A) small phosphatase 2’ |
| ‘NM_006432’ | ‘NPC2’ | ‘Niemann-Pick disease, type C2’ |
| ‘NM_002444’ | ‘MSN’ | ‘moesin’ |
| ‘NM_018054’ | ‘ARHGAP17’ | ‘Rho GTPase activating protein 17’ |
| ‘NM_018267’ | ‘H2AFJ’ | ‘H2A histone family, member J’ |
| ‘NM_003223’ | ‘TFAP4’ | ‘transcription factor AP-4 (activating enhancer binding |
| | | protein 4)’ |
| ‘Contig50799_RC’ | ‘STK4’ | ‘serine/threonine kinase 4’ |
| ‘Contig42649_RC’ | ‘TEP1’ | ‘telomerase-associated protein 1’ |
| ‘NM_002055’ | ‘GFAP’ | ‘glial fibrillary acidic protein’ |
| ‘AL049449’ | ‘GAB1’ | ‘GRB2-associated binding protein 1’ |
| ‘NM_006472’ | ‘TXNIP’ | ‘thioredoxin interacting protein’ |
| ‘NM_000213’ | ‘ITGB4’ | ‘integrin, beta 4’ |
| ‘Contig45443_RC’ | ‘INSR’ | ‘insulin receptor’ |
| ‘NM_018660’ | ‘ZNF395’ | ‘zinc finger protein 395’ |
| ‘NM_000385’ | ‘AQP1’ | ‘aquaporin 1 (Colton blood group)’ |
| ‘NM_004592’ | ‘SFRS8’ | ‘splicing factor, arginine/serine-rich 8 (suppressor-of- |
| | | white-apricot homolog, Drosophila)’ |
| ‘NM_004183’ | ‘BEST1’ | ‘bestrophin 1’ |
| ‘AL122071’ | ‘SLC16A9’ | ‘solute carrier family 16, member 9 (monocarboxylic acid |
| | | transporter 9)’ |
| ‘NM_005106’ | ‘DLEC1’ | ‘deleted in lung and esophageal cancer 1’ |
| ‘NM_000327’ | ‘ROM1’ | ‘retinal outer segment membrane protein 1’ |
| ‘Contig39129_RC’ | ‘AFF1’ | ‘AF4/FMR2 family, member 1’ |
| ‘D79991’ | ‘NUP188’ | ‘nucleoporin 188 kDa’ |
| ‘NM_001381’ | ‘DOK1’ | ‘docking protein 1, 62 kDa (downstream of tyrosine |
| | | kinase 1)’ |
| ‘NM_005296’ | ‘LPAR4’ | ‘lysophosphatidic acid receptor 4’ |
| ‘NM_000552’ | ‘VWF’ | ‘von Willebrand factor’ |
| ‘NM_002966’ | ‘S100A10’ | ‘S100 calcium binding protein A10’ |
| ‘NM_005935’ | ‘AFF1’ | ‘AF4/FMR2 family, member 1’ |
| ‘NM_001540’ | ‘HSPB1’ | ‘heat shock 27 kDa protein 1’ |
| ‘NM_007311’ | ‘TSPO’ | ‘translocator protein (18 kDa)’ |
| ‘NM_012385’ | ‘NUPR1’ | ‘nuclear protein, transcriptional regulator, 1’ |
| ‘Contig51940_RC’ | ‘GABPA’ | ‘GA binding protein transcription factor, alpha subunit |
| | | 60 kDa’ |
| ‘Contig34348_RC’ | ‘NCAM1’ | ‘neural cell adhesion molecule 1’ |
| ‘Contig46590’ | ‘C5orf56’ | ‘chromosome 5 open reading frame 56’ |
| ‘AK000216’ | ‘ZDHHC3’ | ‘zinc finger, DHHC-type containing 3’ |
| ‘NM_000290’ | ‘PGAM2’ | ‘phosphoglycerate mutase 2 (muscle)’ |
| ‘NM_000592’ | ‘C4B’ | ‘complement component 4B (Chido blood group)’ |
| ‘NM_003945’ | ‘ATP6V0E1’ | ‘ATPase, H+ transporting, lysosomal 9 kDa, V0 subunit e1’ |
| ‘NM_004964’ | ‘HDAC1’ | ‘histone deacetylase 1’ |
| ‘NM_004028’ | ‘AQP4’ | ‘aquaporin 4’ |
| ‘AL133117’ | ‘THOC2’ | ‘THO complex 2’ |
| ‘NM_004585’ | ‘RARRES3’ | ‘retinoic acid receptor responder (tazarotene induced) 3’ |
| ‘NM_002859’ | ‘PXN’ | ‘paxillin’ |
| ‘NM_000121’ | ‘EPOR’ | ‘erythropoietin receptor’ |
| ‘NM_001154’ | ‘ANXA5’ | ‘annexin A5’ |
| ‘NM_002905’ | ‘RDH5’ | ‘retinol dehydrogenase 5 (11-cis/9-cis)’ |
| ‘NM_013994’ | ‘DDR1’ | ‘discoidin domain receptor tyrosine kinase 1’ |
| ‘NM_018089’ | ‘ANKZF1’ | ‘ankyrin repeat and zinc finger domain containing 1’ |
| ‘Contig38645_RC’ | ‘AKT2’ | ‘v-akt murine thymoma viral oncogene homolog 2’ |
| ‘Contig55984_RC’ | ‘RELL1’ | ‘RELT-like 1’ |
| ‘NM_018214’ | ‘LRRC1’ | ‘leucine rich repeat containing 1’ |
| ‘NM_016733’ | ‘LIMK2’ | ‘LIM domain kinase 2’ |
| ‘NM_016323’ | ‘HERC5’ | ‘hect domain and RLD 5’ |
| ‘NM_004817’ | ‘TJP2’ | ‘tight junction protein 2 (zona occludens 2)’ |
| ‘AL133108’ | ‘ZFHX3’ | ‘zinc finger homeobox 3’ |
| ‘NM_001954’ | ‘DDR1’ | ‘discoidin domain receptor tyrosine kinase 1’ |
| ‘NM_001885’ | ‘CRYAB’ | ‘crystallin, alpha B’ |
| ‘NM_016201’ | ‘AMOTL2’ | ‘angiomotin like 2’ |
| ‘NM_013448’ | ‘BAZ1A’ | ‘bromodomain adjacent to zinc finger domain, 1A’ |
| ‘NM_006795’ | ‘EHD1’ | ‘EH-domain containing 1’ |
| ‘NM_006623’ | ‘PHGDH’ | ‘phosphoglycerate dehydrogenase’ |
| ‘NM_003051’ | ‘SLC16A1’ | ‘solute carrier family 16, member 1 (monocarboxylic acid |
| | | transporter 1)’ |
| ‘NM_006307’ | ‘SRPX’ | ‘sushi-repeat-containing protein, X-linked’ |
| ‘AB007964’ | ‘KIAA0495’ | ‘KIAA0495’ |
| ‘NM_018458’ | ‘WWC3’ | ‘WWC family member 3’ |
| ‘NM_000714’ | ‘TSPO’ | ‘translocator protein (18 kDa)’ |
| ‘Contig55734_RC’ | ‘XPNPEP3’ | ‘X-prolyl aminopeptidase (aminopeptidase P) 3, putative’ |
| ‘NM_000292’ | ‘PHKA2’ | ‘phosphorylase kinase, alpha 2 (liver)’ |
| ‘NM_007018’ | ‘CEP110’ | ‘centrosomal protein 110 kDa’ |
| ‘Contig678_RC’ | ‘VEZF1’ | ‘vascular endothelial zinc finger 1’ |
| ‘NM_014020’ | ‘TMEM176B’ | ‘transmembrane protein 176B’ |
| ‘NM_002035’ | ‘KDSR’ | ‘3-ketodihydrosphingosine reductase’ |
| ‘NM_004301’ | ‘ACTL6A’ | ‘actin-like 6A’ |
| ‘NM_007359’ | ‘CASC3’ | ‘cancer susceptibility candidate 3’ |
| ‘AW573085_RC’ | ‘C10orf105’ | ‘chromosome 10 open reading frame 105’ |
| ‘Contig52320’ | ‘KDSR’ | ‘3-ketodihydrosphingosine reductase’ |
| ‘NM_002880’ | ‘RAF1’ | ‘v-raf-1 murine leukemia viral oncogene homolog 1’ |
| ‘NM_004058’ | ‘CAPS’ | ‘calcyphosine’ |
| ‘NM_003244’ | ‘TGIF1’ | ‘TGFB-induced factor homeobox 1’ |
| ‘Contig1778_RC’ | ‘ANKRD36BP1’ | ‘ankyrin repeat domain 36B pseudogene 1’ |
| ‘NM_080737’ | ‘SYTL4’ | ‘synaptotagmin-like 4’ |
| ‘ENST00000300680’ | ‘TTC36’ | ‘tetratricopeptide repeat domain 36’ |
| ‘NM_022060’ | ‘ABHD4’ | ‘abhydrolase domain containing 4’ |
| ‘NM_022152’ | ‘TMBIM1’ | ‘transmembrane BAX inhibitor motif containing 1’ |
| ‘NM_024516’ | ‘C16orf53’ | ‘chromosome 16 open reading frame 53’ |
| ‘NM_022776’ | ‘OSBPL11’ | ‘oxysterol binding protein-like 11’ |
| ‘NM_032369’ | ‘HVCN1’ | ‘hydrogen voltage-gated channel 1’ |
| ‘ENST00000222983’ | ‘AZGP1P1’ | ‘alpha-2-glycoprotein 1, zinc-binding pseudogene 1’ |
| ‘ENST00000295772’ | ‘AZGP1P1’ | ‘similar to histone H3.3B’ |
| ‘NM_024513’ | ‘FYCO1’ | ‘FYVE and coiled-coil domain containing 1’ |
| ‘NM_024633’ | ‘C14orf139’ | ‘chromosome 14 open reading frame 139’ |
| ‘NM_024309’ | ‘TNIP2’ | ‘TNFAIP3 interacting protein 2’ |
| ‘NM_025202’ | ‘EFHD1’ | ‘EF-hand domain family, member D1’ |
| ‘AK057713’ | ‘FAM114A1’ | ‘family with sequence similarity 114, member A1’ |
| ‘AK056227’ | ‘KCTD11’ | ‘potassium channel tetramerisation domain containing 11’ |
| ‘NM_032800’ | ‘C1orf198’ | ‘chromosome 1 open reading frame 198’ |
| ‘AB011126’ | ‘FNBP1’ | ‘formin binding protein 1’ |
| ‘AB058716’ | ‘LZTS2’ | ‘leucine zipper, putative tumor suppressor 2’ |
| ‘NM_000247’ | ‘MICA’ | ‘MHC class I polypeptide-related sequence A’ |
| ‘NM_022370’ | ‘ROBO3’ | ‘roundabout, axon guidance receptor, homolog 3 |
| | | (Drosophila)’ |
| ‘NM_021831’ | ‘AGBL5’ | ‘ATP/GTP binding protein-like 5’ |
| ‘NM_001755’ | ‘CBFB’ | ‘core-binding factor, beta subunit’ |
| ‘NM_024959’ | ‘SLC24A6’ | ‘solute carrier family 24 (sodium/potassium/calcium |
| | | exchanger), member 6’ |
| ‘NM_021126’ | ‘MPST’ | ‘mercaptopyruvate sulfurtransferase’ |
| ‘Contig52114_RC’ | ‘PPAPDC1B’ | ‘phosphatidic acid phosphatase type 2 domain |
| | | containing 1B’ |
| ‘NM_022365’ | ‘DNAJC1’ | ‘DnaJ (Hsp40) homolog, subfamily C, member 1’ |
| ‘NM_147187’ | ‘TNFRSF10B’ | ‘tumor necrosis factor receptor superfamily, member 10b’ |
| ‘NM_152637’ | ‘METTL7B’ | ‘methyltransferase like 7B’ |
| ‘NM_002221’ | ‘ITPKB’ | ‘inositol 1,4,5-trisphosphate 3-kinase B’ |
| ‘NM_032204’ | ‘ASCC2’ | ‘activating signal cointegrator 1 complex subunit 2’ |
| ‘NM_004759’ | ‘MAPKAPK2’ | ‘mitogen-activated protein kinase-activated protein |
| | | kinase 2’ |
| ‘NM_173852’ | ‘KRTCAP2’ | ‘keratinocyte associated protein 2’ |
| ‘NM_004339’ | ‘PTTG1IP’ | ‘pituitary tumor-transforming 1 interacting protein’ |
| ‘NM_013450’ | ‘BAZ2B’ | ‘bromodomain adjacent to zinc finger domain, 2B’ |
| ‘NM_001084’ | ‘PLOD3’ | ‘procollagen-lysine, 2-oxoglutarate 5-dioxygenase 3’ |
| ‘NM_001145’ | ‘ANG’ | ‘angiogenin, ribonuclease, RNase A family, 5’ |
| ‘NM_024729’ | ‘MYH14’ | ‘myosin, heavy chain 14, non-muscle’ |
| ‘NM_004422’ | ‘DVL2’ | ‘dishevelled, dsh homolog 2 (Drosophila)’ |
| ‘NM_175058’ | ‘PLEKHA7’ | ‘pleckstrin homology domain containing, family A |
| | | member 7’ |
| ‘NM_015079’ | ‘TBC1D2B’ | ‘TBC1 domain family, member 2B’ |
| ‘NM_002230’ | ‘JUP’ | ‘junction plakoglobin’ |
| ‘NM_004926’ | ‘ZFP36L1’ | ‘zinc finger protein 36, C3H type-like 1’ |
| ‘NM_024657’ | ‘MORC4’ | ‘MORC family CW-type zinc finger 4’ |
| ‘NM_020119’ | ‘ZC3HAV1’ | ‘zinc finger CCCH-type, antiviral 1’ |
| ‘NM_018090’ | ‘NECAP2’ | ‘NECAP endocytosis associated 2’ |
| ‘NM_000391’ | ‘TPP1’ | ‘tripeptidyl peptidase I’ |
| ‘NM_004840’ | ‘ARHGEF6’ | ‘Rac/Cdc42 guanine nucleotide exchange factor (GEF) 6’ |
| ‘NM_130439’ | ‘MXI1’ | ‘MAX interactor 1’ |
| ‘NM_052932’ | ‘TMEM123’ | ‘transmembrane protein 123’ |
| ‘NM_004273’ | ‘CHST3’ | ‘carbohydrate (chondroitin 6) sulfotransferase 3’ |
| ‘NM_025158’ | ‘RUFY1’ | ‘RUN and FYVE domain containing 1’ |
| ‘NM_015997’ | ‘C1orf66’ | ‘chromosome 1 open reading frame 66’ |
| ‘NM_006289’ | ‘TLN1’ | ‘talin 1’ |
| ‘NM_080739’ | ‘C20orf141’ | ‘chromosome 20 open reading frame 141’ |
| ‘NM_007293’ | ‘C4A’ | ‘complement component 4A (Rodgers blood group)’ |
| ‘NM_005675’ | ‘DGCR6’ | ‘DiGeorge syndrome critical region gene 6’ |
| ‘NM_177989’ | ‘ACTL6A’ | ‘actin-like 6A’ |
| ‘NM_005120’ | ‘MED12’ | ‘mediator complex subunit 12’ |
| ‘NM_001185’ | ‘AZGP1’ | ‘alpha-2-glycoprotein 1, zinc-binding’ |
| ‘NM_016937’ | ‘POLA1’ | ‘polymerase (DNA directed), alpha 1, catalytic subunit’ |
| ‘NM_181714’ | ‘LCA5’ | ‘Leber congenital amaurosis 5’ |
| ‘NM_014045’ | ‘LRP10’ | ‘low density lipoprotein receptor-related protein 10’ |
| ‘NM_017606’ | ‘ZNF395’ | ‘zinc finger protein 395’ |
| ‘NM_002673’ | ‘PLXNB1’ | ‘plexin B1’ |
| ‘NM_014604’ | ‘TAX1BP3’ | ‘Tax1 (human T-cell leukemia virus type I) binding |
| | | protein 3’ |
| ‘NM_007300’ | ‘BRCA1’ | ‘breast cancer 1, early onset’ |
| ‘NM_017707’ | ‘ASAP3’ | ‘ArfGAP with SH3 domain, ankyrin repeat and PH |
| | | domain 3’ |
| ‘NM_052897’ | ‘MBD6’ | ‘methyl-CpG binding domain protein 6’ |
| ‘NM_015680’ | ‘C2orf24’ | ‘chromosome 2 open reading frame 24’ |
| ‘NM_016397’ | ‘TH1L’ | ‘TH1-like (Drosophila)’ |
| ‘NM_030961’ | ‘TRIM56’ | ‘tripartite motif-containing 56’ |
| ‘NM_130798’ | ‘SNAP23’ | ‘synaptosomal-associated protein, 23 kDa’ |
| ‘NM_018995’ | ‘MOV10L1’ | ‘Mov10l1, Moloney leukemia virus 10-like 1, homolog |
| | | (mouse)’ |
| ‘NM_017664’ | ‘ANKRD10’ | ‘ankyrin repeat domain 10’ |
| ‘NM_006877’ | ‘GMPR’ | ‘guanosine monophosphate reductase’ |
| ‘NM_006185’ | ‘NUMA1’ | ‘nuclear mitotic apparatus protein 1’ |
| ‘NM_015920’ | ‘RPS27L’ | ‘ribosomal protein S27-like’ |
| ‘NM_182755’ | ‘ZNF438’ | ‘zinc finger protein 438’ |
| ‘NM_006373’ | ‘VAT1’ | ‘vesicle amine transport protein 1 homolog |
| | | (T. californica)’ |
| ‘NM_006736’ | ‘DNAJB2’ | ‘DnaJ (Hsp40) homolog, subfamily B, member 2’ |
| ‘NM_021975’ | ‘RELA’ | ‘v-rel reticuloendotheliosis viral oncogene homolog A |
| | | (avian)’ |
| ‘NM_006076’ | ‘AGFG2’ | ‘ArfGAP with FG repeats 2’ |
| ‘NM_014871’ | ‘PAN2’ | ‘PAN2 poly(A) specific ribonuclease subunit homolog |
| | | (S. cerevisiae)’ |
| ‘NM_024310’ | ‘PLEKHF1’ | ‘pleckstrin homology domain containing, family F (with |
| | | FYVE domain) member 1’ |
| ‘NM_022487’ | ‘DCLRE1C’ | ‘DNA cross-link repair 1C (PSO2 homolog, S. cerevisiae)’ |
| ‘NM_148954’ | ‘PSMB9’ | ‘proteasome (prosome, macropain) subunit, beta type, 9 |
| | | (large multifunctional peptidase 2)’ |
| ‘NM_024334’ | ‘TMEM43’ | ‘transmembrane protein 43’ |
| ‘NM_015374’ | ‘SUN2’ | ‘Sad1 and UNC84 domain containing 2’ |
| ‘NM_181696’ | ‘PRDX1’ | ‘peroxiredoxin 1’ |
| ‘NM_014437’ | ‘SLC39A1’ | ‘solute carrier family 39 (zinc transporter), member 1’ |
| ‘NM_145059’ | ‘FUK’ | ‘fucokinase’ |
| ‘NM_004816’ | ‘FAM189A2’ | ‘family with sequence similarity 189, member A2’ |
| ‘NM_002015’ | ‘FOXO1’ | ‘forkhead box O1’ |
| ‘NM_005569’ | ‘LIMK2’ | ‘LIM domain kinase 2’ |
| ‘NM_153186’ | ‘KANK1’ | ‘KN motif and ankyrin repeat domains 1’ |
| ‘NM_032709’ | ‘PYROXD2’ | ‘pyridine nucleotide-disulphide oxidoreductase domain 2’ |
| ‘NM_174896’ | ‘C1orf162’ | ‘chromosome 1 open reading frame 162’ |
| ‘NM_016376’ | ‘ANKFY1’ | ‘ankyrin repeat and FYVE domain containing 1’ |
| ‘NM_181715’ | ‘CRTC2’ | ‘CREB regulated transcription coactivator 2’ |
| ‘NM_032691’ | ‘CRTC2’ | ‘hypothetical LOC84777’ |
| ‘NM_005157’ | ‘ABL1’ | ‘c-abl oncogene 1, receptor tyrosine kinase’ |
| ‘NM_153265’ | ‘EML3’ | ‘echinoderm microtubule associated protein like 3’ |
| ‘NM_017617’ | ‘NOTCH1’ | ‘Notch homolog 1, translocation-associated (Drosophila)’ |
| ‘NM_019613’ | ‘WDR45L’ | ‘WDR45-like’ |
| ‘NM_178450’ | ‘MARCH3’ | ‘membrane-associated ring finger (C3HC4) 3’ |
| ‘NM_015107’ | ‘PHF8’ | ‘PHD finger protein 8’ |
| ‘NM_004568’ | ‘SERPINB6’ | ‘serpin peptidase inhibitor, clade B (ovalbumin), |
| | | member 6’ |
| ‘NM_152586’ | ‘USP54’ | ‘ubiquitin specific peptidase 54’ |
| ‘NM_000302’ | ‘PLOD1’ | ‘procollagen-lysine 1, 2-oxoglutarate 5-dioxygenase 1’ |
| ‘NM_014521’ | ‘SH3BP4’ | ‘SH3-domain binding protein 4’ |
| ‘NM_032992’ | ‘CASP6’ | ‘caspase 6, apoptosis-related cysteine peptidase’ |
| ‘NM_004739’ | ‘MTA2’ | ‘metastasis associated 1 family, member 2’ |
| ‘NM_016272’ | ‘TOB2’ | ‘transducer of ERBB2, 2’ |
| ‘NM_021149’ | ‘COTL1’ | ‘coactosin-like 1 (Dictyostelium)’ |
| ‘NM_148961’ | ‘OTOS’ | ‘otospiralin’ |
| ‘NM_005631’ | ‘SMO’ | ‘smoothened homolog (Drosophila)’ |
| ‘NM_012257’ | ‘HBP1’ | ‘HMG-box transcription factor 1’ |
| ‘NM_000419’ | ‘ITGA2B’ | ‘integrin, alpha 2b (platelet glycoprotein IIb of IIb/IIIa |
| | | complex, antigen CD41)’ |
| ‘NM_173653’ | ‘SLC9A9’ | ‘solute carrier family 9 (sodium/hydrogen exchanger), |
| | | member 9’ |
| ‘NM_014300’ | ‘SEC11A’ | ‘SEC11 homolog A (S. cerevisiae)’ |
| ‘NM_033178’ | ‘DUX4’ | ‘double homeobox, 4’ |
| ‘NM_173165’ | ‘NFATC3’ | ‘nuclear factor of activated T-cells, cytoplasmic, |
| | | calcineurin-dependent 3’ |
| ‘HSS00020637’ | ‘SMAD4’ | ‘SMAD family member 4’ |
| ‘hCT2283962’ | ‘SMAD4’ | ‘adaptor-related protein complex 1, sigma 2 subunit |
| | | pseudogene’ |
| ‘AK024268’ | ‘ZNF766’ | ‘zinc finger protein 766’ |
| ‘BC006127’ | ‘SRGAP1’ | ‘SLIT-ROBO Rho GTPase activating protein 1’ |
| ‘HSS00171739’ | ‘EPM2AIP1’ | ‘EPM2A (laforin) interacting protein 1’ |
| ‘AL157459’ | ‘CBX2’ | ‘chromobox homolog 2 (Pc class homolog, Drosophila)’ |
| ‘NM_004510’ | ‘SP110’ | ‘SP110 nuclear body protein’ |
| ‘NM_001002029’ | ‘C4B’ | ‘complement component 4B (Chido blood group)’ |
| ‘XM_371630’ | ‘RPS27’ | ‘ribosomal protein S27’ |
| ‘AI939423’ | ‘OTOS’ | ‘otospiralin’ |
| ‘ENST00000336156’ | ‘C22orf9’ | ‘chromosome 22 open reading frame 9’ |
| ‘NM_003051’ | ‘SLC16A1’ | ‘solute carrier family 16, member 1 (monocarboxylic acid |
| | | transporter 1)’ |
| ‘NM_003842’ | ‘TNFRSF10B’ | ‘tumor necrosis factor receptor superfamily, member 10b’ |
| ‘K02403_sat’ | ‘C4A’ | ‘complement component 4A (Rodgers blood group)’ |
| ‘NM_000204_sat’ | ‘CFI’ | ‘complement factor I’ |
| ‘NM_004964’ | ‘HDAC1’ | ‘histone deacetylase 1’ |
| ‘NM_014437’ | ‘SLC39A1’ | ‘solute carrier family 39 (zinc transporter), member 1’ |
| ‘NM_001085_sat’ | ‘SERPINA3’ | ‘serpin peptidase inhibitor, clade A (alpha-1 |
| | | antiproteinase, antitrypsin), member 3’ |

TABLE 3
Correlated Genes for −BioAge

| RefSeq Gene | | |
|---|---|---|
| Transcript | Gene | |
| Identification | Symbol | Gene Name/Description |
| ‘Contig20623_RC’ | ‘FREM3’ | ‘FRAS1 related extracellular matrix 3’ |
| ‘NM_000830’ | ‘GRIK1’ | ‘glutamate receptor, ionotropic, kainate 1’ |
| ‘NM_001683’ | ‘ATP2B2’ | ‘ATPase, Ca++ transporting, plasma membrane 2’ |
| ‘NM_005737 | ‘ARL4C’ | ‘ADP-ribosylation factor-like 4C’ |
| ‘NM_004338’ | ‘C18orf1’ | ‘chromosome 18 open reading frame 1’ |
| ‘AK000827’ | ‘C18orf1’ | ‘hypothetical LOC65996’ |
| ‘NM_006670’ | ‘TPBG’ | ‘trophoblast glycoprotein’ |
| ‘NM_006228’ | ‘PNOC’ | ‘prepronociceptin’ |
| ‘Contig16588_RC’ | ‘CBLN4’ | ‘cerebellin 4 precursor’ |
| ‘NM_000621’ | ‘HTR2A’ | ‘5-hydroxytryptamine (serotonin) receptor 2A’ |
| ‘NM_012329’ | ‘MMD’ | ‘monocyte to macrophage differentiation-associated’ |
| ‘NM_018092’ | ‘NETO2’ | ‘neuropilin (NRP) and tolloid (TLL)-like 2’ |
| ‘NM_015417’ | ‘SPEF1’ | ‘sperm flagellar 1’ |
| ‘NM_005731’ | ‘ARPC2’ | ‘actin related protein 2/3 complex, subunit 2, 34 kDa’ |
| ‘NM_014309’ | ‘RBM9’ | ‘RNA binding motif protein 9’ |
| ‘NM_002744’ | ‘PRKCZ’ | ‘protein kinase C, zeta’ |
| ‘NM_005458’ | ‘GABBR2’ | ‘gamma-aminobutyric acid (GABA) B receptor, 2’ |
| ‘Contig53277_RC’ | ‘ADRBK2’ | ‘adrenergic, beta, receptor kinase 2’ |
| ‘NM_005759’ | ‘ABI2’ | ‘abl-interactor 2’ |
| ‘NM_020178’ | ‘CA10’ | ‘carbonic anhydrase X’ |
| ‘AB037810’ | ‘SIPA1L2’ | ‘signal-induced proliferation-associated 1 like 2’ |
| ‘NM_003381’ | ‘VIP’ | ‘vasoactive intestinal peptide’ |
| ‘NM_004772’ | ‘C5orf13’ | ‘chromosome 5 open reading frame 13’ |
| ‘NM_007026’ | ‘DUSP14’ | ‘dual specificity phosphatase 14’ |
| ‘Contig31754_RC’ | ‘SLITRK1’ | ‘SLIT and NTRK-like family, member 1’ |
| ‘NM_001800’ | ‘CDKN2D’ | ‘cyclin-dependent kinase inhibitor 2D (p19, inhibits |
| | | CDK4)’ |
| ‘NM_001117’ | ‘ADCYAP1’ | ‘adenylate cyclase activating polypeptide 1 (pituitary)’ |
| ‘NM_014592’ | ‘KCNIP1’ | ‘Kv channel interacting protein 1’ |
| ‘NM_001152’ | ‘SLC25A5’ | ‘solute carrier family 25 (mitochondrial carrier; adenine |
| | | nucleotide translocator), member 5’ |
| ‘Contig39157_RC’ | ‘PCP4L1’ | ‘Purkinje cell protein 4 like 1’ |
| ‘Contig44867_RC’ | ‘RGS4’ | ‘regulator of G-protein signaling 4’ |
| ‘NM_002010’ | ‘FGF9’ | ‘fibroblast growth factor 9 (glia-activating factor)’ |
| ‘NM_001048’ | ‘SST’ | ‘somatostatin’ |
| ‘NM_006366’ | ‘CAP2’ | ‘CAP, adenylate cyclase-associated protein, 2 (yeast)’ |
| ‘NM_006428’ | ‘MRPL28’ | ‘mitochondrial ribosomal protein L28’ |
| ‘NM_003558’ | ‘PIP5K1B’ | ‘phosphatidylinositol-4-phosphate 5-kinase, type I, beta’ |
| ‘AB020672’ | ‘MYO16’ | ‘myosin XVI’ |
| ‘NM_000725’ | ‘CACNB3’ | ‘calcium channel, voltage-dependent, beta 3 subunit’ |
| ‘Contig38529_RC’ | ‘XKR4’ | ‘XK, Kell blood group complex subunit-related family, |
| | | member 4’ |
| ‘NM_016522’ | ‘NTM’ | ‘neurotrimin’ |
| ‘NM_014902’ | ‘DLGAP4’ | ‘discs, large (Drosophila) homolog-associated protein 4’ |
| ‘AB002314’ | ‘FRMPD4’ | ‘FERM and PDZ domain containing 4’ |
| ‘NM_004929’ | ‘CALB1’ | ‘calbindin 1, 28 kDa’ |
| ‘Contig55770_RC’ | ‘GSK3B’ | ‘glycogen synthase kinase 3 beta’ |
| ‘NM_004796’ | ‘NRXN3’ | ‘neurexin 3’ |
| ‘NM_006240’ | ‘PPEF1’ | ‘protein phosphatase, EF-hand calcium binding domain 1’ |
| ‘NM_018650’ | ‘MARK1’ | ‘MAP/microtubule affinity-regulating kinase 1’ |
| ‘Contig15728_RC’ | ‘GRIN2A’ | ‘glutamate receptor, ionotropic, N-methyl D-aspartate 2A’ |
| ‘NM_000756’ | ‘CRH’ | ‘corticotropin releasing hormone’ |
| ‘Contig39045_RC’ | ‘CRH’ | ‘hypothetical protein LOC157503’ |
| ‘Contig20799_RC’ | ‘SPRN’ | ‘shadow of prion protein homolog (zebrafish)’ |
| ‘NM_016231’ | ‘NLK’ | ‘nemo-like kinase’ |
| ‘NM_000818’ | ‘GAD2’ | ‘glutamate decarboxylase 2 (pancreatic islets and brain, |
| | | 65 kDa)’ |
| ‘Contig44694_RC’ | ‘ZDHHC8’ | ‘zinc finger, DHHC-type containing 8’ |
| ‘NM_001744’ | ‘CAMK4’ | ‘calcium/calmodulin-dependent protein kinase IV’ |
| ‘NM_003305’ | ‘TRPC3’ | ‘transient receptor potential cation channel, subfamily C, |
| | | member 3’ |
| ‘NM_016588’ | ‘NRN1’ | ‘neuritin 1’ |
| ‘NM_005343’ | ‘HRAS’ | ‘v-Ha-ras Harvey rat sarcoma viral oncogene homolog’ |
| ‘NM_016073’ | ‘HRAS’ | ‘hepatoma-derived growth factor, related protein 3’ |
| ‘NM_005739’ | ‘RASGRP1’ | ‘RAS guanyl releasing protein 1 (calcium and DAG- |
| | | regulated)’ |
| ‘NM_005614’ | ‘RHEB’ | ‘Ras homolog enriched in brain’ |
| ‘Contig35333_RC’ | ‘EMID2’ | ‘EMI domain containing 2’ |
| ‘Contig42274_RC’ | ‘NRIP3’ | ‘nuclear receptor interacting protein 3’ |
| ‘NM_000729’ | ‘CCK’ | ‘cholecystokinin’ |
| ‘NM_013251’ | ‘TAC3’ | ‘tachykinin 3’ |
| ‘NM_020445’ | ‘ACTR3B’ | ‘ARP3 actin-related protein 3 homolog B (yeast)’ |
| ‘NM_018013’ | ‘SOBP’ | ‘sine oculis binding protein homolog (Drosophila)’ |
| ‘NM_018442’ | ‘DCAF6’ | ‘DDB1 and CUL4 associated factor 6’ |
| ‘NM_018639’ | ‘WSB2’ | ‘WD repeat and SOCS box-containing 2’ |
| ‘NM_014038’ | ‘BZW2’ | ‘basic leucine zipper and W2 domains 2’ |
| ‘Contig39732_RC’ | ‘FGF14’ | ‘fibroblast growth factor 14’ |
| ‘NM_004436’ | ‘ENSA’ | ‘endosulfine alpha’ |
| ‘NM_007275’ | ‘TUSC2’ | ‘tumor suppressor candidate 2’ |
| ‘NM_004551’ | ‘NDUFS3’ | ‘NADH dehydrogenase (ubiquinone) Fe—S protein 3, |
| | | 30 kDa (NADH-coenzyme Q reductase)’ |
| ‘Contig34644_RC’ | ‘RIMS1’ | ‘regulating synaptic membrane exocytosis 1’ |
| ‘NM_007066’ | ‘PKIG’ | ‘protein kinase (cAMP-dependent, catalytic) inhibitor |
| | | gamma’ |
| ‘Contig35526_RC’ | ‘C18orf10’ | ‘chromosome 18 open reading frame 10’ |
| ‘Contig46176_RC’ | ‘FBXW7’ | ‘F-box and WD repeat domain containing 7’ |
| ‘NM_001709’ | ‘BDNF’ | ‘brain-derived neurotrophic factor’ |
| ‘AB029029’ | ‘MYT1L’ | ‘myelin transcription factor 1-like’ |
| ‘Contig55448_RC’ | ‘MAGI1’ | ‘membrane associated guanylate kinase, WW and PDZ |
| | | domain containing 1’ |
| ‘NM_006334’ | ‘OLFM1’ | ‘olfactomedin 1’ |
| ‘NM_012202’ | ‘GNG3’ | ‘guanine nucleotide binding protein (G protein), gamma 3’ |
| ‘NM_006477’ | ‘RASL10A’ | ‘RAS-like, family 10, member A’ |
| ‘NM_004546’ | ‘NDUFB2’ | ‘NADH dehydrogenase (ubiquinone) 1 beta subcomplex, |
| | | 2, 8 kDa’ |
| ‘NM_014618’ | ‘DBC1’ | ‘deleted in bladder cancer 1’ |
| ‘Contig31424_RC’ | ‘C6orf154’ | ‘chromosome 6 open reading frame 154’ |
| ‘NM_000717’ | ‘CA4’ | ‘carbonic anhydrase IV’ |
| ‘Contig64477’ | ‘CA4’ | ‘hypothetical locus LOC401237’ |
| ‘NM_006003’ | ‘UQCRFS1’ | ‘ubiquinol-cytochrome c reductase, Rieske iron-sulfur |
| | | polypeptide 1’ |
| ‘NM_006221’ | ‘PIN1’ | ‘peptidylprolyl cis/trans isomerase, NIMA-interacting 1’ |
| ‘Contig53713_RC’ | ‘CASK’ | ‘calcium/calmodulin-dependent serine protein kinase |
| | | (MAGUK family)’ |
| ‘NM_006224’ | ‘PITPNA’ | ‘phosphatidylinositol transfer protein, alpha’ |
| ‘Contig8885_RC’ | ‘CYCS’ | ‘cytochrome c, somatic’ |
| ‘NM_015361’ | ‘R3HDM1’ | ‘R3H domain containing 1’ |
| ‘AB018292’ | ‘DDN’ | ‘dendrin’ |
| ‘NM_018176’ | ‘LGI2’ | ‘leucine-rich repeat LGI family, member 2’ |
| ‘NM_006176’ | ‘NRGN’ | ‘neurogranin (protein kinase C substrate, RC3)’ |
| ‘NM_004114’ | ‘FGF13’ | ‘fibroblast growth factor 13’ |
| ‘NM_002846’ | ‘PTPRN’ | ‘protein tyrosine phosphatase, receptor type, N’ |
| ‘NM_014191’ | ‘SCN8A’ | ‘sodium channel, voltage gated, type VIII, alpha subunit’ |
| ‘Contig42930_RC’ | ‘EXT1’ | ‘exostoses (multiple) 1’ |
| ‘NM_002719’ | ‘PPP2R5C’ | ‘protein phosphatase 2, regulatory subunit B″; gamma |
| | | isoform’ |
| ‘AB075824’ | ‘TMEM132D’ | ‘transmembrane protein 132D’ |
| ‘Contig39594_RC’ | ‘NRXN3’ | ‘neurexin 3’ |
| ‘NM_032495’ | ‘HOPX’ | ‘HOP homeobox’ |
| ‘AB051517’ | ‘ZYG11B’ | ‘zyg-11 homolog B (C. elegans)’ |
| ‘NM_024074’ | ‘TMEM38A’ | ‘transmembrane protein 38A’ |
| ‘AB067499’ | ‘CCDC85A’ | ‘coiled-coil domain containing 85A’ |
| ‘AL713702’ | ‘FAM19A1’ | ‘family with sequence similarity 19 (chemokine (C-C |
| | | motif)-like), member A1’ |
| ‘NM_130773’ | ‘CNTNAP5’ | ‘contactin associated protein-like 5’ |
| ‘NM_030978’ | ‘ARPC5L’ | ‘actin related protein 2/3 complex, subunit 5-like’ |
| ‘ENST00000301382’ | ‘HSD11B1L’ | ‘hydroxysteroid (11-beta) dehydrogenase 1-like’ |
| ‘NM_022823’ | ‘FNDC4’ | ‘fibronectin type III domain containing 4’ |
| ‘NM_080723’ | ‘NRSN1’ | ‘neurensin 1’ |
| ‘Contig48486_RC’ | ‘MAGI1’ | ‘membrane associated guanylate kinase, WW and PDZ |
| | | domain containing 1’ |
| ‘NM_024645’ | ‘ZMAT4’ | ‘zinc finger, matrin type 4’ |
| ‘NM_024709’ | ‘C1orf115’ | ‘chromosome 1 open reading frame 115’ |
| ‘NM_138339’ | ‘GPR26’ | ‘G protein-coupled receptor 26’ |
| ‘AL512695’ | ‘DOK6’ | ‘docking protein 6’ |
| ‘ENST00000256973’ | ‘DOK6’ | ‘neugrin, neurite outgrowth associated pseudogene’ |
| ‘NM_031909’ | ‘C1QTNF4’ | ‘C1q and tumor necrosis factor related protein 4’ |
| ‘AF085867’ | ‘ABI2’ | ‘abl-interactor 2’ |
| ‘NM_020645’ | ‘NRIP3’ | ‘nuclear receptor interacting protein 3’ |
| ‘NM_080552’ | ‘SLC32A1’ | ‘solute carrier family 32 (GABA vesicular transporter), |
| | | member 1’ |
| ‘AL050004’ | ‘HMGCS1’ | ‘3-hydroxy-3-methylglutaryl-Coenzyme A synthase 1 |
| | | (soluble)’ |
| ‘NM_000738’ | ‘CHRM1’ | ‘cholinergic receptor, muscarinic 1’ |
| ‘NM_133445’ | ‘GRIN3A’ | ‘glutamate receptor, ionotropic, N-methyl-D-aspartate 3A’ |
| ‘BC012203’ | ‘FAM71E1’ | ‘family with sequence similarity 71, member E1’ |
| ‘AK057693’ | ‘CTNND1’ | ‘catenin (cadherin-associated protein), delta 1’ |
| ‘NM_138391’ | ‘TMEM183A’ | ‘transmembrane protein 183A’ |
| ‘NM_006370’ | ‘VTI1B’ | ‘vesicle transport through interaction with t-SNAREs |
| | | homolog 1B (yeast)’ |
| ‘NM_182488’ | ‘USP12’ | ‘ubiquitin specific peptidase 12’ |
| ‘NM_178539’ | ‘FAM19A2’ | ‘family with sequence similarity 19 (chemokine (C-C |
| | | motif)-like), member A2’ |
| ‘NM_177964’ | ‘LYPD6B’ | ‘LY6/PLAUR domain containing 6B’ |
| ‘NM_181644’ | ‘MFSD4’ | ‘major facilitator superfamily domain containing 4’ |
| ‘NM_178124’ | ‘CXorf40A’ | ‘chromosome X open reading frame 40A’ |
| ‘NM_153214’ | ‘FBLN7’ | ‘fibulin 7’ |
| ‘NM_152479’ | ‘TTC9B’ | ‘tetratricopeptide repeat domain 9B’ |
| ‘NM_006222’ | ‘PIN1L’ | ‘peptidylprolyl cis/trans isomerase, NIMA-interacting 1- |
| | | like (pseudogene)’ |
| ‘NM_004717’ | ‘DGKI’ | ‘diacylglycerol kinase, iota’ |
| ‘NM_153773’ | ‘C21orf99’ | ‘cancer-testis SP-1’ |
| ‘NM_022549’ | ‘FEZ1’ | ‘fasciculation and elongation protein zeta 1 (zygin I)’ |
| ‘NM_080656’ | ‘CDKN2AIPNL’ | ‘CDKN2A interacting protein N-terminal like’ |
| ‘NM_018462’ | ‘C3orf10’ | ‘chromosome 3 open reading frame 10’ |
| ‘NM_003459’ | ‘SLC30A3’ | ‘solute carrier family 30 (zinc transporter), member 3’ |
| ‘NM_018711’ | ‘SVOP’ | ‘SV2 related protein homolog (rat)’ |
| ‘NM_002236’ | ‘KCNF1’ | ‘potassium voltage-gated channel, subfamily F, member 1’ |
| ‘NM_014322’ | ‘OPN3’ | ‘opsin 3’ |
| ‘NM_005386’ | ‘NNAT’ | ‘neuronatin’ |
| ‘NM_014279’ | ‘OLFM1’ | ‘olfactomedin 1’ |
| ‘NM_001302’ | ‘CORT’ | ‘cortistatin’ |
| ‘NM_153756’ | ‘FNDC5’ | ‘fibronectin type III domain containing 5’ |
| ‘NM_052886’ | ‘MAL2’ | ‘mal, T-cell differentiation protein 2’ |
| ‘NM_015480’ | ‘PVRL3’ | ‘poliovirus receptor-related 3’ |
| ‘NM_021132’ | ‘PPP3CB’ | ‘protein phosphatase 3 (formerly 2B), catalytic subunit, |
| | | beta isoform’ |
| ‘NM_005331’ | ‘HBQ1’ | ‘hemoglobin, theta 1’ |
| ‘NM_033642’ | ‘FGF13’ | ‘fibroblast growth factor 13’ |
| ‘NM_144669’ | ‘GLT1D1’ | ‘glycosyltransferase 1 domain containing 1’ |
| ‘NM_032622’ | ‘LNX1’ | ‘ligand of numb-protein X 1’ |
| ‘NM_018899’ | ‘PCDHAC2’ | ‘protocadherin alpha subfamily C, 2’ |
| ‘NM_152399’ | ‘TMEM155’ | ‘transmembrane protein 155’ |
| ‘NM_152570’ | ‘LINGO2’ | ‘leucine rich repeat and Ig domain containing 2’ |
| ‘NM_080665’ | ‘FDX1L’ | ‘ferredoxin 1-like’ |
| ‘NM_024331’ | ‘TTPAL’ | ‘tocopherol (alpha) transfer protein-like’ |
| ‘NM_015980’ | ‘TTPAL’ | ‘MMP19 protein’ |
| ‘NM_003936’ | ‘CDK5R2’ | ‘cyclin-dependent kinase 5, regulatory subunit 2 (p39)’ |
| ‘NM_006123’ | ‘IDS’ | ‘iduronate 2-sulfatase’ |
| ‘NM_032808’ | ‘LINGO1’ | ‘leucine rich repeat and Ig domain containing 1’ |
| ‘NM_138390’ | ‘TMEM169’ | ‘transmembrane protein 169’ |
| ‘NM_058176’ | ‘HDAC9’ | ‘histone deacetylase 9’ |
| ‘NM_175611’ | ‘GRIK1’ | ‘glutamate receptor, ionotropic, kainate 1’ |
| ‘NM_021956’ | ‘GRIK2’ | ‘glutamate receptor, ionotropic, kainate 2’ |
| ‘NM_015192’ | ‘PLCB1’ | ‘phospholipase C, beta 1 (phosphoinositide-specific)’ |
| ‘NM_021120’ | ‘DLG3’ | ‘discs, large homolog 3 (Drosophila)’ |
| ‘NM_153442’ | ‘GPR26’ | ‘G protein-coupled receptor 26’ |
| ‘NM_001585’ | ‘MPPED1’ | ‘metallophosphoesterase domain containing 1’ |
| ‘NM_003310’ | ‘TSSC1’ | ‘tumor suppressing subtransferable candidate 1’ |
| ‘NM_020546’ | ‘ADCY2’ | ‘adenylate cyclase 2 (brain)’ |
| ‘NM_173641’ | ‘EPHA10’ | ‘EPH receptor A10’ |
| ‘NM_003812’ | ‘ADAM23’ | ‘ADAM metallopeptidase domain 23’ |
| ‘NM_014839’ | ‘ADAM23’ | ‘lipid phosphate phosphatase-related protein type 4’ |
| ‘NM_004080’ | ‘DGKB’ | ‘diacylglycerol kinase, beta 90 kDa’ |
| ‘NM_016466’ | ‘ANKRD39’ | ‘ankyrin repeat domain 39’ |
| ‘NM_005233’ | ‘EPHA3’ | ‘EPH receptor A3’ |
| ‘NM_023071’ | ‘SPATS2’ | ‘spermatogenesis associated, serine-rich 2’ |
| ‘NM_000815’ | ‘GABRD’ | ‘gamma-aminobutyric acid (GABA) A receptor, delta’ |
| ‘NM_144635’ | ‘FAM131A’ | ‘family with sequence similarity 131, member A’ |
| ‘NM_144720’ | ‘JAKMIP1’ | ‘janus kinase and microtubule interacting protein 1’ |
| ‘NM_014903’ | ‘NAV3’ | ‘neuron navigator 3’ |
| ‘NM_022742’ | ‘CCDC136’ | ‘coiled-coil domain containing 136’ |
| ‘NM_170734’ | ‘BDNF’ | ‘brain-derived neurotrophic factor’ |
| ‘NM_018400’ | ‘SCN3B’ | ‘sodium channel, voltage-gated, type III, beta’ |
| ‘NM_032041’ | ‘NCALD’ | ‘neurocalcin delta’ |
| ‘NM_006539’ | ‘CACNG3’ | ‘calcium channel, voltage-dependent, gamma subunit 3’ |
| ‘NM_181804’ | ‘PKIG’ | ‘protein kinase (cAMP-dependent, catalytic) inhibitor |
| | | gamma’ |
| ‘NM_178423’ | ‘HDAC9’ | ‘histone deacetylase 9’ |
| ‘NM_018900’ | ‘PCDHA1’ | ‘protocadherin alpha 1’ |
| ‘NM_017854’ | ‘TMEM160’ | ‘transmembrane protein 160’ |
| ‘NM_002849’ | ‘PTPRR’ | ‘protein tyrosine phosphatase, receptor type, R’ |
| ‘NM_054033’ | ‘FKBP1B’ | ‘FK506 binding protein 1B, 12.6 kDa’ |
| ‘NM_004798’ | ‘KIF3B’ | ‘kinesin family member 3B’ |
| ‘NM_182598’ | ‘C8orf79’ | ‘chromosome 8 open reading frame 79’ |
| ‘NM_002071’ | ‘GNAL’ | ‘guanine nucleotide binding protein (G protein), alpha |
| | | activating activity polypeptide, olfactory type’ |
| ‘NM_152679’ | ‘SLC10A4’ | ‘solute carrier family 10 (sodium/bile acid cotransporter |
| | | family), member 4’ |
| ‘NM_019854’ | ‘PRMT8’ | ‘protein arginine methyltransferase 8’ |
| ‘NM_025072’ | ‘PTGES2’ | ‘prostaglandin E synthase 2’ |
| ‘NM_002924’ | ‘RGS7’ | ‘regulator of G-protein signaling 7’ |
| ‘NM_032503’ | ‘MCHR2’ | ‘melanin-concentrating hormone receptor 2’ |
| ‘NM_152890’ | ‘COL24A1’ | ‘collagen, type XXIV, alpha 1’ |
| ‘NM_005613’ | ‘RGS4’ | ‘regulator of G-protein signaling 4’ |
| ‘NM_006259’ | ‘PRKG2’ | ‘protein kinase, cGMP-dependent, type II’ |
| ‘NM_020416’ | ‘PPP2R2C’ | ‘protein phosphatase 2 (formerly 2A), regulatory subunit |
| | | B, gamma isoform’ |
| ‘NM_152721’ | ‘DOK6’ | ‘docking protein 6’ |
| ‘AK057925’ | ‘CDKN2AIPNL’ | ‘CDKN2A interacting protein N-terminal like’ |
| ‘BC025996’ | ‘CDKN2AIPNL’ | ‘glucuronidase, beta pseudogene’ |
| ‘HSS00131174’ | ‘CDKN2AIPNL’ | ‘hypothetical LOC100132839’ |
| ‘AL122093’ | ‘CDKN2AIPNL’ | ‘actin, gamma-like’ |
| ‘XM_060309’ | ‘OR2T34’ | ‘olfactory receptor, family 2, subfamily T, member 34’ |
| ‘XM_209601’ | ‘OR2T34’ | ‘hypothetical LOC100192379’ |
| ‘HSS00293550’ | ‘C13orf36’ | ‘chromosome 13 open reading frame 36’ |
| ‘BC037245’ | ‘C13orf36’ | ‘hypothetical LOC100126784’ |
| ‘AK095178’ | ‘C13orf36’ | ‘hypothetical LOC728730’ |
| ‘AK091086’ | ‘C11orf87’ | ‘chromosome 11 open reading frame 87’ |
| ‘BC030087’ | ‘C11orf87’ | ‘hypothetical protein LOC375196’ |
| ‘BC032913’ | ‘C11orf87’ | ‘hypothetical gene supported by BC032913; BC048425’ |
| ‘hCT1970512.1’ | ‘UBE2L6’ | ‘ubiquitin-conjugating enzyme E2L 6’ |
| ‘HSS00289112’ | ‘NXPH2’ | ‘neurexophilin 2’ |
| ‘BC010612’ | ‘C17orf51’ | ‘chromosome 17 open reading frame 51’ |
| ‘BC041476’ | ‘C17orf51’ | ‘hypothetical protein LOC253962’ |
| ‘AF131741’ | ‘C17orf51’ | ‘hypothetical LOC441052’ |
| ‘NM_002738’ | ‘PRKCB’ | ‘protein kinase C, beta’ |
| ‘NM_016300’ | ‘PRKCB’ | ‘cyclic AMP-regulated phosphoprotein, 21 kD’ |
| ‘BQ011971’ | ‘TOMM22’ | ‘translocase of outer mitochondrial membrane 22 homolog |
| | | (yeast)’ |
| ‘NM_178423’ | ‘HDAC9’ | ‘histone deacetylase 9’ |
| ‘NM_003459’ | ‘SLC30A3’ | ‘solute carrier family 30 (zinc transporter), member 3’ |
| ‘NM_001152’ | ‘SLC25A5’ | ‘solute carrier family 25 (mitochondrial carrier; adenine |
| | | nucleotide translocator), member 5’ |

TABLE 4
Correlated Genes for Inflame

| RefSeq Gene | | |
|---|---|---|
| Transcript | Gene | |
| Identification | Symbol | Gene Name/Description |
| ‘NM_014373’ | ‘GPR160’ | ‘G protein-coupled receptor 160’ |
| ‘NM_016650’ | ‘MS4A4A’ | ‘membrane-spanning 4-domains, subfamily A, member 4’ |
| ‘NM_001562’ | ‘IL18’ | ‘interleukin 18 (interferon-gamma-inducing factor)’ |
| ‘NM_002664’ | ‘PLEK’ | ‘pleckstrin’ |
| ‘NM_018659’ | ‘CYTL1’ | ‘cytokine-like 1’ |
| ‘NM_005461’ | ‘MAFB’ | ‘v-maf musculoaponeurotic fibrosarcoma oncogene |
| | | homolog B (avian)’ |
| ‘NM_005849’ | ‘IGSF6’ | ‘immunoglobulin superfamily, member 6’ |
| ‘NM_002727’ | ‘SRGN’ | ‘serglycin’ |
| ‘NM_019027’ | ‘RBM47’ | ‘RNA binding motif protein 47’ |
| ‘NM_006432’ | ‘NPC2’ | ‘Niemann-Pick disease, type C2’ |
| ‘NM_001774’ | ‘CD37’ | ‘CD37 molecule’ |
| ‘NM_004120’ | ‘GBP2’ | ‘guanylate binding protein 2, interferon-inducible’ |
| ‘NM_000698’ | ‘ALOX5’ | ‘arachidonate 5-lipoxygenase’ |
| ‘NM_001175’ | ‘ARHGDIB’ | ‘Rho GDP dissociation inhibitor (GDI) beta’ |
| ‘NM_002133’ | ‘HMOX1’ | ‘heme oxygenase (decycling) 1’ |
| ‘NM_000129’ | ‘F13A1’ | ‘coagulation factor XIII, A1 polypeptide’ |
| ‘NM_002163’ | ‘IRF8’ | ‘interferon regulatory factor 8’ |
| ‘NM_014146’ | ‘LAT2’ | ‘linker for activation of T cells family, member 2’ |
| ‘NM_000061’ | ‘BTK’ | ‘Bruton agammaglobulinemia tyrosine kinase’ |
| ‘NM_021199’ | ‘SQRDL’ | ‘sulfide quinone reductase-like (yeast)’ |
| ‘NM_000211’ | ‘ITGB2’ | ‘integrin, beta 2 (complement component 3 receptor 3 and |
| | | 4 subunit)’ |
| ‘NM_013352’ | ‘DSE’ | ‘dermatan sulfate epimerase’ |
| ‘NM_018234’ | ‘STEAP3’ | ‘STEAP family member 3’ |
| ‘NM_004877’ | ‘GMFG’ | ‘glia maturation factor, gamma’ |
| ‘NM_012252’ | ‘TFEC’ | ‘transcription factor EC’ |
| ‘NM_016619’ | ‘PLAC8’ | ‘placenta-specific 8’ |
| ‘NM_001645’ | ‘APOC1’ | ‘apolipoprotein C-I’ |
| ‘NM_001081’ | ‘CUBN’ | ‘cubilin (intrinsic factor-cobalamin receptor)’ |
| ‘Contig48208_RC’ | ‘ITPRIPL2’ | ‘inositol 1,4,5-triphosphate receptor interacting protein- |
| | | like 2’ |
| ‘NM_002298’ | ‘LCP1’ | ‘lymphocyte cytosolic protein 1 (L-plastin)’ |
| ‘NM_005565’ | ‘LCP2’ | ‘lymphocyte cytosolic protein 2 (SH2 domain containing |
| | | leukocyte protein of 76 kDa)’ |
| ‘NM_002934’ | ‘RNASE2’ | ‘ribonuclease, RNase A family, 2 (liver, eosinophil- |
| | | derived neurotoxin)’ |
| ‘NM_006889’ | ‘CD86’ | ‘CD86 molecule’ |
| ‘NM_003608’ | ‘GPR65’ | ‘G protein-coupled receptor 65’ |
| ‘NM_003982’ | ‘SLC7A7’ | ‘solute carrier family 7 (cationic amino acid transporter, |
| | | y+ system), member 7’ |
| ‘NM_001066’ | ‘TNFRSF1B’ | ‘tumor necrosis factor receptor superfamily, member 1B’ |
| ‘NM_002648’ | ‘PIM1’ | ‘pim-1 oncogene’ |
| ‘NM_005620’ | ‘S100A11’ | ‘S100 calcium binding protein A11’ |
| ‘NM_004951’ | ‘GPR183’ | ‘G protein-coupled receptor 183’ |
| ‘D86976’ | ‘HMHA1’ | ‘histocompatibility (minor) HA-1’ |
| ‘NM_013385’ | ‘CYTH4’ | ‘cytohesin 4’ |
| ‘NM_002838’ | ‘PTPRC’ | ‘protein tyrosine phosphatase, receptor type, C’ |
| ‘NM_001953’ | ‘TYMP’ | ‘thymidine phosphorylase’ |
| ‘NM_002432’ | ‘MNDA’ | ‘myeloid cell nuclear differentiation antigen’ |
| ‘NM_005213’ | ‘CSTA’ | ‘cystatin A (stefin A)’ |
| ‘NM_002863’ | ‘PYGL’ | ‘phosphorylase, glycogen, liver’ |
| ‘NM_002118’ | ‘HLA-DMB’ | ‘major histocompatibility complex, class II, DM beta’ |
| ‘NM_004355’ | ‘CD74’ | ‘CD74 molecule, major histocompatibility complex, class |
| | | II invariant chain’ |
| ‘NM_006682’ | ‘FGL2’ | ‘fibrinogen-like 2’ |
| ‘NM_006847’ | ‘LILRB4’ | ‘leukocyte immunoglobulin-like receptor, subfamily B |
| | | (with TM and ITIM domains), member 4’ |
| ‘NM_000218’ | ‘KCNQ1’ | ‘potassium voltage-gated channel, KQT-like subfamily, |
| | | member 1’ |
| ‘NM_013439’ | ‘PILRA’ | ‘paired immunoglobtn-like type 2 receptor alpha’ |
| ‘NM_001465’ | ‘FYB’ | ‘FYN binding protein (FYB-120/130)’ |
| ‘NM_007311’ | ‘TSPO’ | ‘translocator protein (18 kDa)’ |
| ‘NM_006834’ | ‘RAB32’ | ‘RAB32, member RAS oncogene family’ |
| ‘NM_018460’ | ‘ARHGAP15’ | ‘Rho GTPase activating protein 15’ |
| ‘NM_001558’ | ‘IL10RA’ | ‘interleukin 10 receptor, alpha’ |
| ‘Contig47221_RC’ | ‘NFATC2’ | ‘nuclear factor of activated T-cells, cytoplasmic, |
| | | calcineurin-dependent 2’ |
| ‘NM_005335’ | ‘HCLS1’ | ‘hematopoietic cell-specific Lyn substrate 1’ |
| ‘NM_001734’ | ‘C1S’ | ‘complement component 1, s subcomponent’ |
| ‘NM_001754’ | ‘RUNX1’ | ‘runt-related transcription factor 1’ |
| ‘NM_000358’ | ‘TGFBI’ | ‘transforming growth factor, beta-induced, 68 kDa’ |
| ‘NM_005873’ | ‘RGS19’ | ‘regulator of G-protein signaling 19’ |
| ‘NM_000591’ | ‘CD14’ | ‘CD14 molecule’ |
| ‘Contig55221_RC’ | ‘CD14’ | ‘hypothetical LOC400043’ |
| ‘NM_000581’ | ‘GPX1’ | ‘glutathione peroxidase 1’ |
| ‘NM_002308’ | ‘LGALS9’ | ‘lectin, galactoside-binding, soluble, 9’ |
| ‘NM_004271’ | ‘LY86’ | ‘lymphocyte antigen 86’ |
| ‘Contig43039_RC’ | ‘ALOX5’ | ‘arachidonate 5-lipoxygenase’ |
| ‘NM_002831’ | ‘PTPN6’ | ‘protein tyrosine phosphatase, non-receptor type 6’ |
| ‘NM_001629’ | ‘ALOX5AP’ | ‘arachidonate 5-lipoxygenase-activating protein’ |
| ‘NM_004513’ | ‘IL16’ | ‘interleukin 16 (lymphocyte chemoattractant factor)’ |
| ‘AK002171’ | ‘TGFBR1’ | ‘transforming growth factor, beta receptor 1’ |
| ‘NM_005360’ | ‘MAF’ | ‘v-maf musculoaponeurotic fibrosarcoma oncogene |
| | | homolog (avian)’ |
| ‘Contig36042_RC’ | ‘PIK3CG’ | ‘phosphoinositide-3-kinase, catalytic, gamma polypeptide’ |
| ‘NM_006871’ | ‘RIPK3’ | ‘receptor-interacting serine-threonine kinase 3’ |
| ‘NM_014029’ | ‘RAC2’ | ‘ras-related C3 botulinum toxin substrate 2 (rho family, |
| | | small GTP binding protein Rac2)’ |
| ‘NM_000636’ | ‘SOD2’ | ‘superoxide dismutase 2, mitochondrial’ |
| ‘Contig51352_RC’ | ‘IKZF1’ | ‘IKAROS family zinc finger 1 (Ikaros)’ |
| ‘NM_000064’ | ‘C3’ | ‘complement component 3’ |
| ‘NM_004688’ | ‘NMI’ | ‘N-myc (and STAT) interactor’ |
| ‘NM_000063’ | ‘C2’ | ‘complement component 2’ |
| ‘NM_021175’ | ‘HAMP’ | ‘hepcidin antimicrobial peptide’ |
| ‘NM_001421’ | ‘ELF4’ | ‘E74-like factor 4 (ets domain transcription factor)’ |
| ‘NM_014395’ | ‘DAPP1’ | ‘dual adaptor of phosphotyrosine and 3-phosphoinositides’ |
| ‘NM_002124’ | ‘HLA-DRB1’ | ‘major histocompatibility complex, class II, DR beta 1’ |
| ‘NM_007268’ | ‘VSIG4’ | ‘V-set and immunoglobulin domain containing 4’ |
| ‘NM_001288’ | ‘CLIC1’ | ‘chloride intracellular channel 1’ |
| ‘NM_015364’ | ‘LY96’ | ‘lymphocyte antigen 96’ |
| ‘NM_019018’ | ‘FAM105A’ | ‘family with sequence similarity 105, member A’ |
| ‘Contig50088_RC’ | ‘ADORA3’ | ‘adenosine A3 receptor’ |
| ‘NM_006053’ | ‘TCIRG1’ | ‘T-cell, immune regulator 1, ATPase, H+ transporting, |
| | | lysosomal V0 subunit A3’ |
| ‘NM_000101’ | ‘CYBA’ | ‘cytochrome b-245, alpha polypeptide’ |
| ‘NM_002661’ | ‘PLCG2’ | ‘phospholipase C, gamma 2 (phosphatidylinositol- |
| | | specific)’ |
| ‘NM_003730’ | ‘RNASET2’ | ‘ribonuclease T2’ |
| ‘NM_016582’ | ‘SLC15A3’ | ‘solute carrier family 15, member 3’ |
| ‘NM_018326’ | ‘GIMAP4’ | ‘GTPase, IMAP family member 4’ |
| ‘NM_001560’ | ‘IL13RA1’ | ‘interleukin 13 receptor, alpha 1’ |
| ‘NM_003332’ | ‘TYROBP’ | ‘TYRO protein tyrosine kinase binding protein’ |
| ‘Contig53952_RC’ | ‘PIK3AP1’ | ‘phosphoinositide-3-kinase adaptor protein 1’ |
| ‘NM_006864’ | ‘LILRB3’ | ‘leukocyte immunoglobulin-like receptor, subfamily B |
| | | (with TM and ITIM domains), member 3’ |
| ‘NM_002659’ | ‘PLAUR’ | ‘plasminogen activator, urokinase receptor’ |
| ‘NM_009587’ | ‘LGALS9’ | ‘lectin, galactoside-binding, soluble, 9’ |
| ‘NM_001225’ | ‘CASP4’ | ‘caspase 4, apoptosis-related cysteine peptidase’ |
| ‘NM_019111’ | ‘HLA-DRA’ | ‘major histocompatibility complex, class II, DR alpha’ |
| ‘NM_003937’ | ‘KYNU’ | ‘kynureninase (L-kynurenine hydrolase)’ |
| ‘NM_000714’ | ‘TSPO’ | ‘translocator protein (18 kDa)’ |
| ‘NM_004847’ | ‘AIF1’ | ‘allograft inflammatory factor 1’ |
| ‘NM_013314’ | ‘BLNK’ | ‘B-cell linker’ |
| ‘NM_001772’ | ‘CD33’ | ‘CD33 molecule’ |
| ‘NM_005874’ | ‘LILRB2’ | ‘leukocyte immunoglobulin-like receptor, subfamily B |
| | | (with TM and ITIM domains), member 2’ |
| ‘NM_003177’ | ‘SYK’ | ‘spleen tyrosine kinase’ |
| ‘NM_000377 | ‘WAS’ | ‘Wiskott-Aldrich syndrome (eczema-thrombocytopenia)’ |
| ‘NM_005628’ | ‘SLC1A5’ | ‘solute carrier family 1 (neutral amino acid transporter), |
| | | member 5’ |
| ‘NM_001814’ | ‘CTSC’ | ‘cathepsin C’ |
| ‘NM_003039’ | ‘SLC2A5’ | ‘solute carrier family 2 (facilitated glucose/fructose |
| | | transporter), member 5’ |
| ‘NM_002350’ | ‘LYN’ | ‘v-yes-1 Yamaguchi sarcoma viral related oncogene |
| | | homolog’ |
| ‘NM_002342’ | ‘LTBR’ | ‘lymphotoxin beta receptor (TNFR superfamily, |
| | | member 3)’ |
| ‘NM_000397’ | ‘CYBB’ | ‘cytochrome b-245, beta polypeptide’ |
| ‘NM_001908’ | ‘CTSB’ | ‘cathepsin B’ |
| ‘NM_005337’ | ‘NCKAP1L’ | ‘NCK-associated protein 1-like’ |
| ‘Contig10690_RC’ | ‘SYK’ | ‘spleen tyrosine kinase’ |
| ‘Contig50728_RC’ | ‘PTAFR’ | ‘platelet-activating factor receptor’ |
| ‘NM_003890’ | ‘FCGBP’ | ‘Fc fragment of IgG binding protein’ |
| ‘NM_005428’ | ‘VAV1’ | ‘vav 1 guanine nucleotide exchange factor’ |
| ‘NM_001733’ | ‘C1R’ | ‘complement component 1, r subcomponent’ |
| ‘NM_016187’ | ‘BIN2’ | ‘bridging integrator 2’ |
| ‘NM_004079’ | ‘CTSS’ | ‘cathepsin S’ |
| ‘NM_012214’ | ‘MGAT4A’ | ‘mannosyl (alpha-1,3-)-glycoprotein beta-1,4-N- |
| | | acetylglucosaminyltransferase, isozyme A’ |
| ‘Contig1030_RC’ | ‘DOCK8’ | ‘dedicator of cytokinesis 8’ |
| ‘NM_006120’ | ‘HLA-DMA’ | ‘major histocompatibility complex, class II, DM alpha’ |
| ‘NM_018594’ | ‘FYB’ | ‘FYN binding protein (FYB-120/130)’ |
| ‘NM_006399’ | ‘BATF’ | ‘basic leucine zipper transcription factor, ATF-like’ |
| ‘NM_002110’ | ‘HCK’ | ‘hemopoietic cell kinase’ |
| ‘NM_003150’ | ‘STAT3’ | ‘signal transducer and activator of transcription 3 (acute- |
| | | phase response factor)’ |
| ‘NM_018965’ | ‘TREM2’ | ‘triggering receptor expressed on myeloid cells 2’ |
| ‘NM_000560’ | ‘CD53’ | ‘CD53 molecule’ |
| ‘Contig33703_RC’ | ‘RASAL3’ | ‘RAS protein activator like 3’ |
| ‘NM_005767’ | ‘LPAR6’ | ‘lysophosphatidic acid receptor 6’ |
| ‘NM_015991’ | ‘C1QA’ | ‘complement component 1, q subcomponent, A chain’ |
| ‘NM_006748’ | ‘SLA’ | ‘Src-like-adaptor’ |
| ‘NM_000632’ | ‘ITGAM’ | ‘integrin, alpha M (complement component 3 receptor 3 |
| | | subunit)’ |
| ‘NM_007161’ | ‘LST1’ | ‘leukocyte specific transcript 1’ |
| ‘NM_005615’ | ‘RNASE6’ | ‘ribonuclease, RNase A family, k6’ |
| ‘NM_006762’ | ‘LAPTM5’ | ‘lysosomal protein transmembrane 5’ |
| ‘AF086130’ | ‘FAM26F’ | ‘family with sequence similarity 26, member F’ |
| ‘AJ420585’ | ‘HLA-DOA’ | ‘major histocompatibility complex, class II, DO alpha’ |
| ‘Contig55671_RC’ | ‘NFATC2’ | ‘nuclear factor of activated T-cells, cytoplasmic, |
| | | calcineurin-dependent 2’ |
| ‘NM_138402’ | ‘SP140L’ | ‘SP140 nuclear body protein-like’ |
| ‘NM_031471’ | ‘FERMT3’ | ‘fermitin family homolog 3 (Drosophila)’ |
| ‘NM_021642’ | ‘FCGR2A’ | ‘Fc fragment of IgG, low affinity IIa, receptor (CD32)’ |
| ‘NM_030956’ | ‘TLR10’ | ‘toll-like receptor 10’ |
| ‘AF116653’ | ‘FYB’ | ‘FYN binding protein (FYB-120/130)’ |
| ‘AK057772’ | ‘SYK’ | ‘spleen tyrosine kinase’ |
| ‘NM_022047’ | ‘DEF6’ | ‘differentially expressed in FDCP 6 homolog (mouse)’ |
| ‘NM_033128’ | ‘SCIN’ | ‘scinderin’ |
| ‘NM_024430’ | ‘PSTPIP2’ | ‘proline-serine-threonine phosphatase interacting |
| | | protein 2’ |
| ‘NM_130446’ | ‘KLHL6’ | ‘kelch-like 6 (Drosophila)’ |
| ‘NM_022136’ | ‘SAMSN1’ | ‘SAM domain, SH3 domain and nuclear localization |
| | | signals 1’ |
| ‘NM_022162’ | ‘NOD2’ | ‘nucleotide-binding oligomerization domain containing 2’ |
| ‘NM_022054’ | ‘KCNK13’ | ‘potassium channel, subfamily K, member 13’ |
| ‘NM_024829’ | ‘PLBD1’ | ‘phospholipase B domain containing 1’ |
| ‘NM_025159’ | ‘CXorf21’ | ‘chromosome X open reading frame 21’ |
| ‘NM_024575’ | ‘TNFAIP8L2’ | ‘tumor necrosis factor, alpha-induced protein 8-like 2’ |
| ‘AK074085’ | ‘WDFY4’ | ‘WDFY family member 4’ |
| ‘NM_138410’ | ‘CMTM7’ | ‘CKLF-like MARVEL transmembrane domain |
| | | containing 7’ |
| ‘NM_022107’ | ‘GPSM3’ | ‘G-protein signaling modulator 3 (AGS3-like, C. elegans)’ |
| ‘NM_006332’ | ‘IFI30’ | ‘interferon, gamma-inducible protein 30’ |
| ‘NM_005720’ | ‘ARPC1B’ | ‘actin related protein 2/3 complex, subunit |
| | | 1B, 41 kDa’ |
| ‘NM_019029’ | ‘CPVL’ | ‘carboxypeptidase, vitellogenic-like’ |
| ‘NM_147780’ | ‘CTSB’ | ‘cathepsin B’ |
| ‘NM_000677’ | ‘ADORA3’ | ‘adenosine A3 receptor’ |
| ‘NM_016543’ | ‘SIGLEC7’ | ‘sialic acid binding Ig-like lectin 7’ |
| ‘NM_024901’ | ‘DENND2D’ | ‘DENN/MADD domain containing 2D’ |
| ‘NM_017817’ | ‘RAB20’ | ‘RAB20, member RAS oncogene family’ |
| ‘NM_002445’ | ‘MSR1’ | ‘macrophage scavenger receptor 1’ |
| ‘NM_018986’ | ‘SH3TC1’ | ‘SH3 domain and tetratricopeptide repeats 1’ |
| ‘NM_000579’ | ‘CCR5’ | ‘chemokine (C-C motif) receptor 5’ |
| ‘NM_000295’ | ‘SERPINA1’ | ‘serpin peptidase inhibitor, clade A (alpha-1 |
| | | antiproteinase, antitrypsin), member 1’ |
| ‘NM_022059’ | ‘CXCL16’ | ‘chemokine (C-X-C motif) ligand 16’ |
| ‘NM_030666’ | ‘SERPINB1’ | ‘serpin peptidase inhibitor, clade B (ovalbumin), |
| | | member 1’ |
| ‘NM_013416’ | ‘NCF4’ | ‘neutrophil cytosolic factor 4, 40 kDa’ |
| ‘NM_002468’ | ‘MYD88’ | ‘myeloid differentiation primary response gene (88)’ |
| ‘NM_002925’ | ‘RGS10’ | ‘regulator of G-protein signaling 10’ |
| ‘NM_003101’ | ‘SOAT1’ | ‘sterol O-acyltransferase 1’ |
| ‘NM_152851’ | ‘MS4A6A’ | ‘membrane-spanning 4-domains, subfamily A, |
| | | member 6A’ |
| ‘NM_015136’ | ‘STAB1’ | ‘stabilin 1’ |
| ‘NM_138444’ | ‘KCTD12’ | ‘potassium channel tetramerisation domain containing 12’ |
| ‘NM_000566’ | ‘FCGR1A’ | ‘Fc fragment of IgG, high affinity Ia, receptor (CD64)’ |
| ‘NM_181720’ | ‘ARHGAP30’ | ‘Rho GTPase activating protein 30’ |
| ‘NM_004244’ | ‘CD163’ | ‘CD163 molecule’ |
| ‘NM_000760’ | ‘CSF3R’ | ‘colony stimulating factor 3 receptor (granulocyte)’ |
| ‘NM_016293’ | ‘BIN2’ | ‘bridging integrator 2’ |
| ‘NM_000578’ | ‘SLC11A1’ | ‘solute carrier family 11 (proton-coupled divalent metal |
| | | ion transporters), member 1’ |
| ‘NM_024599’ | ‘RHBDF2’ | ‘rhomboid 5 homolog 2 (Drosophila)’ |
| ‘NM_022570’ | ‘CLEC7A’ | ‘C-type lectin domain family 7, member A’ |
| ‘NM_153337’ | ‘SNX20’ | ‘sorting nexin 20’ |
| ‘NM_006074’ | ‘TRIM22’ | ‘tripartite motif-containing 22’ |
| ‘NM_022349’ | ‘MS4A6A’ | ‘membrane-spanning 4-domains, subfamily A, |
| | | member 6A’ |
| ‘NM_021777’ | ‘ADAM28’ | ‘ADAM metallopeptidase domain 28’ |
| ‘NM_024832’ | ‘RIN3’ | ‘Ras and Rab interactor 3’ |
| ‘NM_014385’ | ‘SIGLEC7’ | ‘sialic acid binding Ig-like lectin 7’ |
| ‘NM_032782’ | ‘HAVCR2’ | ‘hepatitis A virus cellular receptor 2’ |
| ‘NM_033130’ | ‘SIGLEC10’ | ‘sialic acid binding Ig-like lectin 10’ |
| ‘NM_181724’ | ‘TMEM119’ | ‘transmembrane protein 119’ |
| ‘NM_002543’ | ‘OLR1’ | ‘oxidized low density lipoprotein (lectin-like) receptor 1’ |
| ‘NM_021706’ | ‘LAIR1’ | ‘leukocyte-associated immunoglobulin-like receptor 1’ |
| ‘NM_014608’ | ‘CYFIP1’ | ‘cytoplasmic FMR1 interacting protein 1’ |
| ‘NM_022141’ | ‘PARVG’ | ‘parvin, gamma’ |
| ‘NM_015660’ | ‘GIMAP2’ | ‘GTPase, IMAP family member 2’ |
| ‘NM_021983’ | ‘HLA-DRB4’ | ‘major histocompatibility complex, class II, DR beta 4’ |
| ‘NM_000507’ | ‘FBP1’ | ‘fructose-1,6-bisphosphatase 1’ |
| ‘NM_004946’ | ‘DOCK2’ | ‘dedicator of cytokinesis 2’ |
| ‘NM_021209’ | ‘NLRC4’ | ‘NLR family, CARD domain containing 4’ |
| ‘NM_007261’ | ‘CD300A’ | ‘CD300a molecule’ |
| ‘NM_014265’ | ‘ADAM28’ | ‘ADAM metallopeptidase domain 28’ |
| ‘NM_000570’ | ‘FCGR3B’ | ‘Fc fragment of IgG, low affinity IIIb, receptor (CD16b)’ |
| ‘NM_018404’ | ‘ADAP2’ | ‘ArfGAP with dual PH domains 2’ |
| ‘NM_003264’ | ‘TLR2’ | ‘toll-like receptor 2’ |
| ‘NM_172247’ | ‘CSF2RA’ | ‘colony stimulating factor 2 receptor, alpha, low-affinity |
| | | (granulocyte-macrophage)’ |
| ‘NM_148170’ | ‘CTSC’ | ‘cathepsin C’ |
| ‘NM_145041’ | ‘TMEM106A’ | ‘transmembrane protein 106A’ |
| ‘NM_000491’ | ‘C1QB’ | ‘complement component 1, q subcomponent, B chain’ |
| ‘NM_006474’ | ‘PDPN’ | ‘podoplanin’ |
| ‘NM_016562’ | ‘TLR7’ | ‘toll-like receptor 7’ |
| ‘NM_000576’ | ‘IL1B’ | ‘interleukin 1, beta’ |
| ‘NM_080921’ | ‘PTPRC’ | ‘protein tyrosine phosphatase, receptor type, C’ |
| ‘NM_000572’ | ‘IL10’ | ‘interleukin 10’ |
| ‘NM_016428’ | ‘ABI3’ | ‘ABI family, member 3’ |
| ‘NM_000803’ | ‘FOLR2’ | ‘folate receptor 2 (fetal)’ |
| ‘NM_002029’ | ‘FPR1’ | ‘formyl peptide receptor 1’ |
| ‘NM_025144’ | ‘ALPK1’ | ‘alpha-kinase 1’ |
| ‘NM_003263’ | ‘TLR1’ | ‘toll-like receptor 1’ |
| ‘NM_006866’ | ‘LILRA2’ | ‘leukocyte immunoglobulin-like receptor, subfamily A |
| | | (with TM domain), member 2’ |
| ‘NM_005779’ | ‘LHFPL2’ | ‘lipoma HMGIC fusion partner-like 2’ |
| ‘NM_001637’ | ‘AOAH’ | ‘acyloxyacyl hydrolase (neutrophil)’ |
| ‘NM_005211’ | ‘CSF1R’ | ‘colony stimulating factor 1 receptor’ |
| ‘NM_000433’ | ‘NCF2’ | ‘neutrophil cytosolic factor 2’ |
| ‘NM_148975’ | ‘MS4A4A’ | ‘membrane-spanning 4-domains, subfamily A, member 4’ |
| ‘NM_174896’ | ‘C1orf162’ | ‘chromosome 1 open reading frame 162’ |
| ‘NM_013258’ | ‘PYCARD’ | ‘PYD and CARD domain containing’ |
| ‘NM_018690’ | ″ | ‘apolipoprotein B48 receptor’ |
| ‘NM_012072’ | ‘CD93’ | ‘CD93 molecule’ |
| ‘NM_002935’ | ‘RNASE3’ | ‘ribonuclease, RNase A family, 3 (eosinophil cationic |
| | | protein)’ |
| ‘NM_004054’ | ‘C3AR1’ | ‘complement component 3a receptor 1’ |
| ‘NM_033295’ | ‘CASP1’ | ‘caspase 1, apoptosis-related cysteine peptidase |
| | | (interleukin 1, beta, convertase)’ |
| ‘NM_021778’ | ‘ADAM28’ | ‘ADAM metallopeptidase domain 28’ |
| ‘NM_003761’ | ‘VAMP8’ | ‘vesicle-associated membrane protein 8 (endobrevin)’ |
| ‘NM_175862’ | ‘CD86’ | ‘CD86 molecule’ |
| ‘NM_016610’ | ‘TLR8’ | ‘toll-like receptor 8’ |
| ‘NM_172369’ | ‘C1QC’ | ‘complement component 1, q subcomponent, C chain’ |
| ‘NM_005202’ | ‘COL8A2’ | ‘collagen, type VIII, alpha 2’ |
| ‘NM_019043’ | ‘APBB1IP’ | ‘amyloid beta (A4) precursor protein-binding, family B, |
| | | member 1 interacting protein’ |
| ‘NM_138715’ | ‘MSR1’ | ‘macrophage scavenger receptor 1’ |
| ‘NM_006678’ | ‘CD300C’ | ‘CD300c molecule’ |
| ‘NM_012335’ | ‘MYO1F’ | ‘myosin IF’ |
| ‘NM_004573’ | ‘PLCB2’ | ‘phospholipase C, beta 2’ |
| ‘NM_021201’ | ‘MS4A7’ | ‘membrane-spanning 4-domains, subfamily A, member 7’ |
| ‘NM_152309’ | ‘PIK3AP1’ | ‘phosphoinositide-3-kinase adaptor protein 1’ |
| ‘NM_004106’ | ‘FCER1G’ | ‘Fc fragment of IgE, high affinity I, receptor for; gamma |
| | | polypeptide’ |
| ‘NM_001295’ | ‘CCR1’ | ‘chemokine (C-C motif) receptor 1’ |
| ‘NM_144658’ | ‘DOCK11’ | ‘dedicator of cytokinesis 11’ |
| ‘NM_172246’ | ‘CSF2RA’ | ‘colony stimulating factor 2 receptor, alpha, low-affinity |
| | | (granulocyte-macrophage)’ |
| ‘NM_022083’ | ‘FAM129A’ | ‘family with sequence similarity 129, member A’ |
| ‘NM_000631’ | ‘NCF4’ | ‘neutrophil cytosolic factor 4, 40 kDa’ |
| ‘NM_024943’ | ‘TMEM156’ | ‘transmembrane protein 156’ |
| ‘NM_130782’ | ‘RGS18’ | ‘regulator of G-protein signaling 18’ |
| ‘NM_001061’ | ‘TBXAS1’ | ‘thromboxane A synthase 1 (platelet)’ |
| ‘NM_005531’ | ‘IFI16’ | ‘interferon, gamma-inducible protein 16’ |
| ‘NM_020041’ | ‘SLC2A9’ | ‘solute carrier family 2 (facilitated glucose transporter), |
| | | member 9’ |
| ‘NM_005755’ | ‘EBI3’ | ‘Epstein-Barr virus induced 3’ |
| ‘NM_173558’ | ‘FGD2’ | ‘FYVE, RhoGEF and PH domain containing 2’ |
| ‘NM_033554’ | ‘HLA-DPA1’ | ‘major histocompatibility complex, class II, DP alpha 1’ |
| ‘NM_020125’ | ‘SLAMF8’ | ‘SLAM family member 8’ |
| ‘hCT1775405.1’ | ‘PRSS3’ | ‘protease, serine, 3’ |
| ‘HSS00212166’ | ‘ANKRD22’ | ‘ankyrin repeat domain 22’ |
| ‘XM_211305’ | ‘C17orf60’ | ‘chromosome 17 open reading frame 60’ |
| ‘NM_001623’ | ‘AIF1’ | ‘allograft inflammatory factor 1’ |
| ‘NM_000569’ | ‘FCGR3A’ | ‘Fc fragment of IgG, low affinity IIIa, receptor (CD16a)’ |
| ‘CB529629’ | ‘FCER1G’ | ‘Fc fragment of IgE, high affinity I, receptor for; gamma |
| | | polypeptide’ |
| ‘BM684049’ | ‘HAMP’ | ‘hepcidin antimicrobial peptide’ |
| ‘NM_001005412’ | ‘FCGR2C’ | ‘Fc fragment of IgG, low affinity IIc, receptor for (CD32)’ |
| ‘hCT34994’ | ‘HLA-DRA’ | ‘major histocompatibility complex, class II, DR alpha’ |
| ‘ENST00000343801’ | ‘CCR5’ | ‘chemokine (C-C motif) receptor 5’ |
| ‘BC073889’ | ‘LGALS9C’ | ‘lectin, galactoside-binding, soluble, 9C’ |
| ‘ENST00000342052’ | ‘TMEM106A’ | ‘transmembrane protein 106A’ |
| ‘AP119873’ | ‘SERPINA1’ | ‘serpin peptidase inhibitor, clade A (alpha-1 |
| | | antiproteinase, antitrypsin), member 1’ |
| ‘NM_001004340’ | ‘FCGR1B’ | ‘Fc fragment of IgG, high affinity Ib, receptor (CD64)’ |
| ‘BQ015859’ | ‘CSTA’ | ‘cystatin A (stefin A)’ |
| ‘NM_021175_sat’ | ‘HAMP’ | ‘hepcidin antimicrobial peptide’ |
| ‘NM_005628’ | ‘SLC1A5’ | ‘solute carrier family 1 (neutral amino acid transporter), |
| | | member 5’ |
| ‘NM_001733_sat’ | ‘C1R’ | ‘complement component 1, r subcomponent’ |
| ‘NM_000295_sat’ | ‘SERPINA1’ | ‘serpin peptidase inhibitor, clade A (alpha-1 |
| | | antiproteinase, antitrypsin), member 1’ |
| ‘NM_001066’ | ‘TNFRSF1B’ | ‘tumor necrosis factor receptor superfamily, member 1B’ |
| ‘NM_001061’ | ‘TBXAS1’ | ‘thromboxane A synthase 1 (platelet)’ |
| ‘NM_003982’ | ‘SLC7A7’ | ‘solute carrier family 7 (cationic amino acid transporter, |
| | | y+ system), member 7’ |
| ‘NM_001734_sat’ | ‘C1S’ | ‘complement component 1, s subcomponent’ |
| ‘NM_000204_sat’ | ‘CFI’ | ‘complement factor I’ |
| ‘NM_003039’ | ‘SLC2A5’ | ‘solute carrier family 2 (facilitated glucose/fructose |
| | | transporter), member 5’ |
| ‘NM_001001290’ | ‘SLC2A9’ | ‘solute carrier family 2 (facilitated glucose transporter), |
| | | member 9’ |
| ‘NM_000578’ | ‘SLC11A1’ | ‘solute carrier family 11 (proton-coupled divalent metal |
| | | ion transporters), member 1’ |

TABLE 5
Correlated Genes for +NdStress

| RefSeq Gene | | |
|---|---|---|
| Transcript | Gene | |
| Identification | SymbolGene | Name/Description |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘NM_005895’ | ‘GOLGA3’ | ‘golgin A3’ |
| ‘HSS00253039’ | ‘PROX2’ | ‘prospero homeobox 2’ |
| ‘NM_012308’ | ‘KDM2A’ | ‘lysine (K)-specific demethylase 2A’ |
| ‘NM_015443’ | ‘KIAA1267’ | ‘KIAA1267’ |
| ‘AB002374’ | ‘CYTSA’ | ‘cytospin A’ |
| ‘NM_025081’ | ‘NYNRIN’ | ‘NYN domain and retroviral integrase containing’ |
| ‘NM_002011’ | ‘FGFR4’ | ‘fibroblast growth factor receptor 4’ |
| ‘NM_015908’ | ‘SRRT’ | ‘serrate RNA effector molecule homolog (Arabidopsis)’ |
| ‘hCT9675.2’ | ‘PHRF1’ | ‘PHD and ring finger domains 1’ |
| ‘Contig45443_RC’ | ‘INSR’ | ‘insulin receptor’ |
| ‘NM_014079’ | ‘KLF15’ | ‘Kruppel-like factor 15’ |
| ‘NM_021639’ | ‘GPBP1L1’ | ‘GC-rich promoter binding protein 1-like 1’ |
| ‘NM_000934’ | ‘SERPINF2’ | ‘serpin peptidase inhibitor, clade F (alpha-2 antiplasmin, |
| | | pigment epithelium derived factor), member 2’ |
| ‘AK093990’ | ‘SERPINF2’ | ‘hypothetical protein LOC284009’ |
| ‘NM_173215’ | ‘NFAT5’ | ‘nuclear factor of activated T-cells 5, tonicity-responsive’ |
| ‘NM_032886’ | ‘RBM14’ | ‘RNA binding motif protein 14’ |
| ‘HSS00143708’ | ‘C10orf104’ | ‘chromosome 10 open reading frame 104’ |
| ‘NM_006541’ | ‘GLRX3’ | ‘glutaredoxin 3’ |
| ‘Contig29362_RC’ | ‘ANKRD13D’ | ‘ankyrin repeat domain 13 family, member D’ |
| ‘NM_014823’ | ‘WNK1’ | ‘WNK lysine deficient protein kinase 1’ |
| ‘NM_032887’ | ‘FAM69B’ | ‘family with sequence similarity 69, member B’ |
| ‘AJ006835’ | ‘SNORA73A’ | ‘small nucleolar RNA, H/ACA box 73A’ |
| ‘HSS00087436’ | ‘ANKRD52’ | ‘ankyrin repeat domain 52’ |
| ‘BC001742’ | ‘ANKRD52’ | ‘hypothetical protein BC001742’ |
| ‘AK025065’ | ‘NMT2’ | ‘N-myristoyltransferase 2’ |
| ‘AK023936’ | ‘HSPA12A’ | ‘heat shock 70 kDa protein 12A’ |
| ‘HSS00276358’ | ‘DNAJB6’ | ‘DnaJ (Hsp40) homolog, subfamily B, member 6’ |
| ‘NM_018346’ | ‘RSAD1’ | ‘radical S-adenosyl methionine domain containing 1’ |
| ‘NM_013325’ | ‘ATG4B’ | ‘ATG4 autophagy related 4 homolog B (S. cerevisiae)’ |
| ‘ENST00000316798’ | ‘ATG4B’ | ‘ATG4 autophagy related 4 homolog B (S. cerevisiae)’ |
| ‘NM_002693’ | ‘POLG’ | ‘polymerase (DNA directed), gamma’ |
| ‘NM_004922’ | ‘SEC24C’ | ‘SEC24 family, member C (S. cerevisiae)’ |
| ‘ENST00000273582’ | ‘KIAA0226’ | ‘KIAA0226’ |
| ‘NM_006232’ | ‘POLR2H’ | ‘polymerase (RNA) II (DNA directed) polypeptide H’ |
| ‘NM_145806’ | ‘ZNF511’ | ‘zinc finger protein 511’ |
| ‘NM_006645’ | ‘STARD10’ | ‘StAR-related lipid transfer (START) domain |
| | | containing 10’ |
| ‘NM_198317’ | ‘KLHL17’ | ‘kelch-like 17 (Drosophila)’ |
| ‘NM_032998’ | ‘DEDD’ | ‘death effector domain containing’ |
| ‘NM_024419’ | ‘PGS1’ | ‘phosphatidylglycerophosphate synthase 1’ |
| ‘NM_133336’ | ‘WHSC1’ | ‘Wolf-Hirschhorn syndrome candidate 1’ |
| ‘NM_033194’ | ‘HSPB9’ | ‘heat shock protein, alpha-crystallin-related, B9’ |
| ‘NM_006145’ | ‘DNAJB1’ | ‘DnaJ (Hsp40) homolog, subfamily B, member 1’ |
| ‘NM_005346’ | ‘HSPA1B’ | ‘heat shock 70 kDa protein 1B’ |
| ‘NM_005345’ | ‘HSPA1A’ | ‘heat shock 70 kDa protein 1A’ |
| ‘NM_006819’ | ‘STIP1’ | ‘stress-induced-phosphoprotein 1’ |
| ‘NM_004199’ | ‘P4HA2’ | ‘prolyl 4-hydroxylase, alpha polypeptide II’ |
| ‘NM_001539’ | ‘DNAJA1’ | ‘DnaJ (Hsp40) homolog, subfamily A, member 1’ |
| ‘NM_012124’ | ‘CHORDC1’ | ‘cysteine and histidine-rich domain (CHORD)- |
| | | containing 1’ |
| ‘NM_001237’ | ‘CCNA2’ | ‘cyclin A2’ |
| ‘NM_005527’ | ‘HSPA1L’ | ‘heat shock 70 kDa protein 1-like’ |
| ‘Contig13488_RC’ | ‘CDKN2AIP’ | ‘CDKN2A interacting protein’ |
| ‘Contig48935_RC’ | ‘SIX4’ | ‘SIX homeobox 4’ |
| ‘NM_032623’ | ‘C4orf49’ | ‘chromosome 4 open reading frame 49’ |
| ‘NM_003797’ | ‘EED’ | ‘embryonic ectoderm development’ |
| ‘X96655’ | ‘SNORD56’ | ‘small nucleolar RNA, C/D box 56’ |
| ‘Contig17556_RC’ | ‘FAM59B’ | ‘family with sequence similarity 59, member B’ |
| ‘AK000229’ | ‘C18orf49’ | ‘chromosome 18 open reading frame 49’ |
| ‘NM_018157’ | ‘RIC8B’ | ‘resistance to inhibitors of cholinesterase 8 homolog B |
| | | (C. elegans)’ |
| ‘AF070587 | ‘CCDC88C’ | ‘coiled-coil domain containing 88C’ |
| ‘NM_058246’ | ‘DNAJB6’ | ‘DnaJ (Hsp40) homolog, subfamily B, member 6’ |
| ‘NM_001269’ | ‘RCC1’ | ‘regulator of chromosome condensation 1’ |
| ‘NM_002896’ | ‘RBM4’ | ‘RNA binding motif protein 4’ |
| ‘NM_003124’ | ‘SPR’ | ‘sepiapterin reductase (7,8-dihydrobiopterin: NADP+ |
| | | oxidoreductase)’ |
| ‘NM_079837’ | ‘BANP’ | ‘BTG3 associated nuclear protein’ |
| ‘NM_017869’ | ‘BANP’ | ‘BTG3 associated nuclear protein’ |
| ‘NM_173510’ | ‘CCDC117’ | ‘coiled-coil domain containing 117’ |
| ‘NM_052957’ | ‘ACRC’ | ‘acidic repeat containing’ |
| ‘NM_182597’ | ‘C7orf53’ | ‘chromosome 7 open reading frame 53’ |
| ‘NM_014664’ | ‘N4BP1’ | ‘NEDD4 binding protein 1’ |
| ‘NM_003161’ | ‘RPS6KB1’ | ‘ribosomal protein S6 kinase, 70 kDa, polypeptide 1’ |
| ‘NM_138278’ | ‘BNIPL’ | ‘BCL2/adenovirus E1B 19 kD interacting protein like’ |
| ‘BC018064’ | ‘BNIPL’ | ‘similar to proteaseome (prosome, macropain) 28 |
| | | subunit, 3’ |
| ‘NM_016507’ | ‘CDK12’ | ‘cyclin-dependent kinase 12’ |
| ‘NM_001807’ | ‘CEL’ | ‘carboxyl ester lipase (bile salt-stimulated lipase)’ |
| ‘NM_001374’ | ‘DNASE1L2’ | ‘deoxyribonuclease I-like 2’ |
| ‘NM_031946’ | ‘AGAP3’ | ‘ArfGAP with GTPase domain, ankyrin repeat and PH |
| | | domain 3’ |
| ‘NM_145718’ | ‘TRAF2’ | ‘TNF receptor-associated factor 2’ |
| ‘NM_022759’ | ‘ENGASE’ | ‘endo-beta-N-acetylglucosaminidase’ |
| ‘NM_014851’ | ‘KLHL21’ | ‘kelch-like 21 (Drosophila)’ |
| ‘NM_014941’ | ‘MORC2’ | ‘MORC family CW-type zinc finger 2’ |
| ‘NM_006328’ | ‘RBM14’ | ‘RNA binding motif protein 14’ |
| ‘NM_022046’ | ‘KLK14’ | ‘kallikrein-related peptidase 14’ |
| ‘AF218021’ | ‘KLK14’ | ‘hypothetical protein LOC100129503’ |
| ‘NM_145045’ | ‘CCDC151’ | ‘coiled-coil domain containing 151’ |
| ‘NM_020062’ | ‘SLC2A4RG’ | ‘SLC2A4 regulator’ |
| ‘NM_001472’ | ‘GAGE2C’ | ‘G antigen 2C’ |
| ‘XM_210035’ | ‘PPP1R3F’ | ‘protein phosphatase 1, regulatory (inhibitor) subunit 3F’ |
| ‘NM_001475’ | ‘GAGE5’ | ‘G antigen 5’ |
| ‘NM_001474’ | ‘GAGE4’ | ‘G antigen 4’ |
| ‘NM_012196’ | ‘GAGE8’ | ‘G antigen 8’ |
| ‘NM_001476’ | ‘GAGE6’ | ‘G antigen 6’ |
| ‘NM_001477’ | ‘GAGE12I’ | ‘G antigen 12I’ |
| ‘NM_021123’ | ‘GAGE7’ | ‘G antigen 7’ |
| ‘U19144’ | ‘GAGE3’ | ‘G antigen 3’ |
| ‘Contig23475_RC’ | ‘MICALL2’ | ‘MICAL-like 2’ |
| ‘NM_024052’ | ‘C17orf39’ | ‘chromosome 17 open reading frame 39’ |
| ‘NM_015714’ | ‘G0S2’ | ‘G0/G1switch 2’ |
| ‘NM_130469’ | ‘JDP2’ | ‘Jun dimerization protein 2’ |
| ‘hCT2316334’ | ‘COL27A1’ | ‘collagen, type XXVII, alpha 1’ |
| ‘AF274938’ | ‘RP9P’ | ‘retinitis pigmentosa 9 pseudogene’ |
| ‘NM_020382’ | ‘SETD8’ | ‘SET domain containing (lysine methyltransferase) 8’ |
| ‘NM_003579’ | ‘RAD54L’ | ‘RAD54-like (S. cerevisiae)’ |
| ‘NM_031894’ | ‘FTHL17’ | ‘ferritin, heavy polypeptide-like 17’ |
| ‘BC034822’ | ‘FTHL17’ | ‘SPR pseudogene’ |
| ‘NM_003298’ | ‘NR2C2’ | ‘nuclear receptor subfamily 2, group C, member 2’ |
| ‘AW269746’ | ‘COX8C’ | ‘cytochrome c oxidase subunit 8C’ |
| ‘AL049397’ | ‘PPPDE1’ | ‘PPPDE peptidase domain containing 1’ |
| ‘NM_015446’ | ‘AHCTF1’ | ‘AT hook containing transcription factor 1’ |
| ‘NM_003400’ | ‘XPO1’ | ‘exportin 1 (CRM1 homolog, yeast)’ |
| ‘NM_025211’ | ‘GKAP1’ | ‘G kinase anchoring protein 1’ |
| ‘AK054864’ | ‘IRF2BP2’ | ‘interferon regulatory factor 2 binding protein 2’ |
| ‘NM_015087’ | ‘SPG20’ | ‘spastic paraplegia 20 (Troyer syndrome)’ |
| ‘NM_017672’ | ‘TRPM7’ | ‘transient receptor potential cation channel, subfamily M, |
| | | member 7’ |
| ‘NM_031435’ | ‘THAP2’ | ‘THAP domain containing, apoptosis associated protein 2’ |
| ‘NM_015358’ | ‘MORC3’ | ‘MORC family CW-type zinc finger 3’ |
| ‘hCT12351.3’ | ‘CWC22’ | ‘CWC22 spliceosome-associated protein homolog |
| | | (S. cerevisiae)’ |
| ‘NM_014382’ | ‘ATP2C1’ | ‘ATPase, Ca++ transporting, type 2C, member 1’ |
| ‘NM_015200’ | ‘PDS5A’ | ‘PDS5, regulator of cohesion maintenance, homolog A |
| | | (S. cerevisiae)’ |
| ‘AK001838’ | ‘NUFIP2’ | ‘nuclear fragile X mental retardation protein interacting |
| | | protein 2’ |
| ‘NM_033087’ | ‘ALG2’ | ‘asparagine-linked glycosylation 2, alpha-1,3- |
| | | mannosyltransferase homolog (S. cerevisiae)’ |
| ‘Contig56959_RC’ | ‘CEBPG’ | ‘CCAAT/enhancer binding protein (C/EBP), gamma’ |
| ‘NM_016303’ | ‘WBP5’ | ‘WW domain binding protein 5’ |
| ‘NM_003403’ | ‘YY1’ | ‘YY1 transcription factor’ |
| ‘hCT1639886.3’ | ‘YY1’ | ‘similar to tumor protein, translationally-controlled 1’ |
| ‘NM_001540’ | ‘HSPB1’ | ‘heat shock 27 kDa protein 1’ |
| ‘NM_006912’ | ‘RIT1’ | ‘Ras-like without CAAX 1’ |
| ‘NM_000917’ | ‘P4HA1’ | ‘prolyl 4-hydroxylase, alpha polypeptide I’ |
| ‘Contig44712_RC’ | ‘GNA13’ | ‘guanine nucleotide binding protein (G protein), alpha 13’ |
| ‘NM_013255’ | ‘MKLN1’ | ‘muskelin 1, intracellular mediator containing kelch |
| | | motifs’ |
| ‘NM_024576’ | ‘OGFRL1’ | ‘opioid growth factor receptor-like 1’ |
| ‘NM_021188’ | ‘ZNF410’ | ‘zinc finger protein 410’ |
| ‘Contig50004_RC’ | ‘ZNF410’ | ‘patched domain containing 3 pseudogene’ |
| ‘NM_002577 | ‘PAK2’ | ‘p21 protein (Cdc42/Rac)-activated kinase 2’ |
| ‘NM_007375’ | ‘TARDBP’ | ‘TAR DNA binding protein’ |
| ‘NM_138720’ | ‘HIST1H2BD’ | ‘histone cluster 1, H2bd’ |
| ‘Contig57239_RC’ | ‘KIAA0114’ | ‘KIAA0114’ |
| ‘NM_020960’ | ‘GPR107’ | ‘G protein-coupled receptor 107’ |
| ‘NM_030962’ | ‘SBF2’ | ‘SET binding factor 2’ |
| ‘AK093779’ | ‘SBF2’ | ‘hypothetical LOC399900’ |
| ‘AK023199’ | ‘C1orf226’ | ‘chromosome 1 open reading frame 226’ |
| ‘NM_015478’ | ‘L3MBTL’ | ‘l(3)mbt-like (Drosophila)’ |
| ‘NM_031902’ | ‘MRPS5’ | ‘mitochondrial ribosomal protein S5’ |
| ‘XM_066760’ | ‘MRPS5’ | ‘hypothetical LOC392556’ |
| ‘AK095149’ | ‘ZXDC’ | ‘ZXD family zinc finger C’ |
| ‘NM_021244’ | ‘RRAGD’ | ‘Ras-related GTP binding D’ |
| ‘NM_001675’ | ‘ATF4’ | ‘activating transcription factor 4 (tax-responsive enhancer |
| | | element B67)’ |
| ‘AK093353’ | ‘ATF4’ | ‘hypothetical LOC390251’ |
| ‘NM_182810’ | ‘ATF4’ | ‘activating transcription factor 4 (tax-responsive enhancer |
| | | element B67)’ |
| ‘NM_000392’ | ‘ABCC2’ | ‘ATP-binding cassette, sub-family C (CFTR/MRP), |
| | | member 2’ |
| ‘NM_012110’ | ‘CHIC2’ | ‘cysteine-rich hydrophobic domain 2’ |
| ‘ENST00000334351’ | ‘PNRC2’ | ‘proline-rich nuclear receptor coactivator 2’ |
| ‘Contig53674_RC’ | ‘GNAS’ | ‘GNAS complex locus’ |
| ‘NM_021649’ | ‘TICAM2’ | ‘toll-like receptor adaptor molecule 2’ |
| ‘AB002443’ | ‘TICAM2’ | ‘toll-like receptor adaptor molecule 2’ |
| ‘HSS00346710’ | ‘HNRPLL’ | ‘heterogeneous nuclear ribonucleoprotein L-like’ |
| ‘HSS00329979’ | ‘PSMB1’ | ‘proteasome (prosome, macropain) subunit, beta type, 1’ |
| ‘Contig31062_RC’ | ‘PSMB1’ | ‘hypothetical LOC100216546’ |
| ‘NM_006459’ | ‘ERLIN1’ | ‘ER lipid raft associated 1’ |
| ‘NM_017782’ | ‘C10orf18’ | ‘chromosome 10 open reading frame 18’ |
| ‘NM_033109’ | ‘PNPT1’ | ‘polyribonucleotide nucleotidyltransferase 1’ |
| ‘NM_014991’ | ‘WDFY3’ | ‘WD repeat and FYVE domain containing 3’ |
| ‘NM_177968’ | ‘PPM1B’ | ‘protein phosphatase 1B (formerly 2C), magnesium- |
| | | dependent, beta isoform’ |
| ‘Contig36432_RC’ | ‘KIAA1958’ | ‘KIAA1958’ |
| ‘NM_012257’ | ‘HBP1’ | ‘HMG-box transcription factor 1’ |
| ‘NM_020193’ | ‘C11orf30’ | ‘chromosome 11 open reading frame 30’ |
| ‘NM_003620’ | ‘PPM1D’ | ‘protein phosphatase 1D magnesium-dependent, delta |
| | | isoform’ |
| ‘NM_018133’ | ‘MSL2’ | ‘male-specific lethal 2 homolog (Drosophila)’ |
| ‘NM_014487’ | ‘ZNF330’ | ‘zinc finger protein 330’ |
| ‘NM_138798’ | ‘MITD1’ | ‘MIT, microtubule interacting and transport, domain |
| | | containing 1’ |
| ‘hCT2285874’ | ‘MITD1’ | ‘similar to hCG1820375’ |
| ‘NM_022333’ | ‘TIAL1’ | ‘TIA1 cytotoxic granule-associated RNA binding protein- |
| | | like 1’ |
| ‘AL049449’ | ‘GAB1’ | ‘GRB2-associated binding protein 1’ |
| ‘AB011090’ | ‘MGA’ | ‘MAX gene associated’ |
| ‘AK055661’ | ‘ZBTB34’ | ‘zinc finger and BTB domain containing 34’ |
| ‘NM_024631’ | ‘C11orf61’ | ‘chromosome 11 open reading frame 61’ |
| ‘NM_152792’ | ‘ASPRV1’ | ‘aspartic peptidase, retroviral-like 1’ |
| ‘NM_015885’ | ‘PCF11’ | ‘PCF11, cleavage and polyadenylation factor subunit, |
| | | homolog (S. cerevisiae)’ |
| ‘NM_145796’ | ‘POGZ’ | ‘pogo transposable element with ZNF domain’ |
| ‘NM_003718’ | ‘CDK13’ | ‘cyclin-dependent kinase 13’ |
| ‘NM_016261’ | ‘TUBD1’ | ‘tubulin, delta 1’ |
| ‘ENST00000284765’ | ‘C4orf47’ | ‘chromosome 4 open reading frame 47’ |
| ‘NM_005197’ | ‘FOXN3’ | ‘forkhead box N3’ |
| ‘NM_017936’ | ‘SMEK1’ | ‘SMEK homolog 1, suppressor of mek1 (Dictyostelium)’ |
| ‘NM_001329’ | ‘CTBP2’ | ‘C-terminal binding protein 2’ |
| ‘NM_016593’ | ‘CYP39A1’ | ‘cytochrome P450, family 39, subfamily A, polypeptide 1’ |
| ‘Contig46158_RC’ | ‘SOS1’ | ‘son of sevenless homolog 1 (Drosophila)’ |
| ‘NM_139168’ | ‘SFRS12’ | ‘splicing factor, arginine/serine-rich 12’ |
| ‘NM_152519’ | ‘C2orf67’ | ‘chromosome 2 open reading frame 67’ |
| ‘NM_005359’ | ‘SMAD4’ | ‘SMAD family member 4’ |
| ‘NM_018061’ | ‘PRPF38B’ | ‘PRP38 pre-mRNA processing factor 38 (yeast) domain |
| | | containing B’ |
| ‘NM_006625’ | ‘SFRS13A’ | ‘splicing factor, arginine/serine-rich 13A’ |
| ‘NM_173473’ | ‘C10orf104’ | ‘chromosome 10 open reading frame 104’ |
| ‘Contig53629_RC’ | ‘SOCS4’ | ‘suppressor of cytokine signaling 4’ |
| ‘AK054894’ | ‘MED13’ | ‘mediator complex subunit 13’ |
| ‘AL833463’ | ‘MED13’ | ‘hypothetical protein LOC283658’ |
| ‘NM_052937’ | ‘PCMTD1’ | ‘protein-L-isoaspartate (D-aspartate) O-methyltransferase |
| | | domain containing 1’ |
| ‘NM_005857’ | ‘ZMPSTE24’ | ‘zinc metallopeptidase (STE24 homolog, S. cerevisiae)’ |
| ‘NM_153365’ | ‘TAPT1’ | ‘transmembrane anterior posterior transformation 1’ |
| ‘HSS00126953’ | ‘TMX1’ | ‘thioredoxin-related transmembrane protein 1’ |
| ‘NM_004555’ | ‘NFATC3’ | ‘nuclear factor of activated T-cells, cytoplasmic, |
| | | calcineurin-dependent 3’ |
| ‘NM_024523’ | ‘GCC1’ | ‘GRIP and coiled-coil domain containing 1’ |
| ‘HSS00092615’ | ‘RBM7’ | ‘RNA binding motif protein 7’ |
| ‘NM_017880’ | ‘C2orf42’ | ‘chromosome 2 open reading frame 42’ |
| ‘NM_002486’ | ‘NCBP1’ | ‘nuclear cap binding protein subunit 1, 80 kDa’ |
| ‘NM_016277’ | ‘RAB23’ | ‘RAB23, member RAS oncogene family’ |
| ‘NM_022840’ | ‘METTL4’ | ‘methyltransferase like 4’ |
| ‘NM_005901’ | ‘SMAD2’ | ‘SMAD family member 2’ |
| ‘NM_005927 | ‘MFAP3’ | ‘microfibrillar-associated protein 3’ |
| ‘NM_004275’ | ‘MED20’ | ‘mediator complex subunit 20’ |
| ‘Contig51158_RC’ | ‘AP4E1’ | ‘adaptor-related protein complex 4, epsilon 1 subunit’ |
| ‘NM_018976’ | ‘SLC38A2’ | ‘solute carrier family 38, member 2’ |
| ‘NM_018573’ | ‘SLC38A2’ | ‘solute carrier family 38, member 2’ |
| ‘NM_018976’ | ‘SLC38A2’ | ‘solute carrier family 38, member 2’ |
| ‘NM_014950’ | ‘ZBTB1’ | ‘zinc finger and BTB domain containing 1’ |
| ‘Contig56768_RC’ | ‘SLC5A3’ | ‘solute carrier family 5 (sodium/myo-inositol |
| | | cotransporter), member 3’ |
| ‘NM_032476’ | ‘MRPS6’ | ‘mitochondrial ribosomal protein S6’ |
| ‘Contig1034_RC’ | ‘YY1’ | ‘YY1 transcription factor’ |
| ‘NM_014345’ | ‘ZNF318’ | ‘zinc finger protein 318’ |
| ‘NM_014071’ | ‘NCOA6’ | ‘nuclear receptor coactivator 6’ |
| ‘NM_032120’ | ‘C7orf64’ | ‘chromosome 7 open reading frame 64’ |
| ‘NM_019041’ | ‘MTRF1L’ | ‘mitochondrial translational release factor 1-like’ |
| ‘NM_006973’ | ‘ZNF32’ | ‘zinc finger protein 32’ |
| ‘HSS00217006’ | ‘ANKRD19’ | ‘ankyrin repeat domain 19 pseudogene’ |
| ‘NM_004380’ | ‘CREBBP’ | ‘CREB binding protein’ |
| ‘Contig30995_RC’ | ‘PSMD6’ | ‘proteasome (prosome, macropain) 26S subunit, non- |
| | | ATPase, 6’ |
| ‘NM_001952’ | ‘E2F6’ | ‘E2F transcription factor 6’ |
| ‘AL049782’ | ‘E2F6’ | ‘hypothetical gene CG012’ |
| ‘NM_033111’ | ‘N4BP2L2’ | ‘NEDD4 binding protein 2-like 2’ |
| ‘Contig17475_RC’ | ‘CKLF’ | ‘chemokine-like factor’ |
| ‘NM_153694’ | ‘SYCP3’ | ‘synaptonemal complex protein 3’ |
| ‘AK055378’ | ‘MSL1’ | ‘male-specific lethal 1 homolog (Drosophila)’ |
| ‘NM_100486’ | ‘WAC’ | ‘WW domain containing adaptor with coiled-coil’ |
| ‘NM_018703’ | ‘RBBP6’ | ‘retinoblastoma binding protein 6’ |
| ‘NM_018366’ | ‘CNO’ | ‘cappuccino homolog (mouse)’ |
| ‘NM_020861’ | ‘ZBTB2’ | ‘zinc finger and BTB domain containing 2’ |
| ‘NM_000026’ | ‘ADSL’ | ‘adenylosuccinate lyase’ |
| ‘NM_032763’ | ‘ADSL’ | ‘hypothetical protein MGC16142’ |
| ‘NM_018036’ | ‘ATG2B’ | ‘ATG2 autophagy related 2 homolog B (S. cerevisiae)’ |
| ‘NM_032875’ | ‘FBXL20’ | ‘F-box and leucine-rich repeat protein 20’ |
| ‘NM_018169’ | ‘C12orf35’ | ‘chromosome 12 open reading frame 35’ |
| ‘NM_014928’ | ‘OTUD4’ | ‘OTU domain containing 4’ |
| ‘Contig57056_RC’ | ‘ZBTB38’ | ‘zinc finger and BTB domain containing 38’ |
| ‘NM_003663’ | ‘CGGBP1’ | ‘CGG triplet repeat binding protein 1’ |
| ‘NM_005802’ | ‘TOPORS’ | ‘topoisomerase I binding, arginine/serine-rich’ |
| ‘NM_153244’ | ‘C10orf111’ | ‘chromosome 10 open reading frame 111’ |
| ‘NM_016643’ | ‘ZNF771’ | ‘zinc finger protein 771’ |
| ‘NM_015148’ | ‘PASK’ | ‘PAS domain containing serine/threonine kinase’ |
| ‘HSS00269962’ | ‘C15orf62’ | ‘chromosome 15 open reading frame 62’ |
| ‘Contig5954_RC’ | ‘ZGLP1’ | ‘zinc finger, GATA-like protein 1’ |
| ‘NM_018277’ | ‘TCP10L’ | ‘t-complex 10 (mouse)-like’ |
| ‘BC004544’ | ‘CYHR1’ | ‘cysteine/histidine-rich 1’ |
| ‘NM_017924’ | ‘C14orf119’ | ‘chromosome 14 open reading frame 119’ |
| ‘NM_024537’ | ‘CARS2’ | ‘cysteinyl-tRNA synthetase 2, mitochondrial (putative)’ |
| ‘NM_020385’ | ‘REXO4’ | ‘REX4, RNA exonuclease 4 homolog (S. cerevisiae)’ |
| ‘Contig27827_RC’ | ‘TMEM81’ | ‘transmembrane protein 81’ |
| ‘Contig51020_RC’ | ‘TADA2B’ | ‘transcriptional adaptor 2B’ |
| ‘Contig38273_RC’ | ‘MSTO1’ | ‘misato homolog 1 (Drosophila)’ |
| ‘NM_144606’ | ‘FLCN’ | ‘folliculin’ |
| ‘AL050061’ | ‘FLCN’ | ‘hypothetical protein LOC157562’ |
| ‘AF086402’ | ‘VPRBP’ | ‘Vpr (HIV-1) binding protein’ |
| ‘HSS00018326’ | ‘VPRBP’ | ‘hypothetical protein LOC100128437’ |
| ‘NM_181305’ | ‘MRPL52’ | ‘mitochondrial ribosomal protein L52’ |
| ‘NM_017432’ | ‘PTOV1’ | ‘prostate tumor overexpressed 1’ |
| ‘Contig52705_RC’ | ‘CREBBP’ | ‘CREB binding protein’ |
| ‘NM_012083’ | ‘FRAT2’ | ‘frequently rearranged in advanced T-cell lymphomas 2’ |
| ‘AB037753’ | ‘FBXO42’ | ‘F-box protein 42’ |
| ‘NM_022034’ | ‘CUZD1’ | ‘CUB and zona pellucida-like domains 1’ |
| ‘NM_152452’ | ‘IGF1R’ | ‘insulin-like growth factor 1 receptor’ |
| ‘NM_032909’ | ‘ZCCHC14’ | ‘zinc finger, CCHC domain containing 14’ |
| ‘NM_015144’ | ‘ZCCHC14’ | ‘zinc finger, CCHC domain containing 14’ |
| ‘NM_018715’ | ‘RCC2’ | ‘regulator of chromosome condensation 2’ |
| ‘NM_012408’ | ‘ZMYND8’ | ‘zinc finger, MYND-type containing 8’ |
| ‘AK091150’ | ‘ZMYND8’ | ‘hypothetical LOC651250’ |
| ‘NM_144997’ | ‘FLCN’ | ‘folliculin’ |
| ‘Contig32050_RC’ | ‘WDR76’ | ‘WD repeat domain 76’ |
| ‘Contig38744_RC’ | ‘WDR76’ | ‘hypothetical protein LOC338620’ |
| ‘XM_087642’ | ‘C5orf48’ | ‘chromosome 5 open reading frame 48’ |
| ‘NM_014270’ | ‘SLC7A9’ | ‘solute carrier family 7 (cationic amino acid transporter, |
| | | y+ system), member 9’ |
| ‘NM_014270’ | ‘SLC7A9’ | ‘solute carrier family 7 (cationic amino acid transporter, |
| | | y+ system), member 9’ |
| ‘AF274937’ | ‘C7orf60’ | ‘chromosome 7 open reading frame 60’ |
| ‘NM_007222’ | ‘ZHX1’ | ‘zinc fingers and homeoboxes 1’ |
| ‘BM977381’ | ‘PAPOLA’ | ‘poly(A) polymerase alpha’ |
| ‘NM_032765’ | ‘TRIM52’ | ‘tripartite motif-containing 52’ |
| ‘NM_024643’ | ‘FAM164C’ | ‘family with sequence similarity 164, member C’ |
| ‘NM_000337’ | ‘SGCD’ | ‘sarcoglycan, delta (35 kDa dystrophin-associated |
| | | glycoprotein)’ |
| ‘AK055913’ | ‘SLC5A3’ | ‘solute carrier family 5 (sodium/myo-inositol |
| | | cotransporter), member 3’ |

TABLE 6
Correlated Genes for -NdStress

| RefSeq Gene | | |
|---|---|---|
| Transcript | Gene | |
| Identification | Symbol | Gene Name/Description |
| ‘NM_012260’ | ‘HACL1’ | ‘2-hydroxyacyl-CoA lyase 1’ |
| ‘NM_006468’ | ‘POLR3C’ | ‘polymerase (RNA) III (DNA directed) polypeptide C |
| | | (62 kD)’ |
| ‘NM_002139’ | ‘RBMX’ | ‘RNA binding motif protein, X-linked’ |
| ‘NM_025234’ | ‘WDR61’ | ‘WD repeat domain 61’ |
| ‘NM_002915’ | ‘RFC3’ | ‘replication factor C (activator 1) 3, 38 kDa’ |
| ‘NM_016004’ | ‘IFT52’ | ‘intraflagellar transport 52 homolog (Chlamydomonas)’ |
| ‘NM_006559’ | ‘KHDRBS1’ | ‘KH domain containing, RNA binding, signal transduction |
| | | associated 1’ |
| ‘NM_016468’ | ‘COX16’ | ‘COX16 cytochrome c oxidase assembly homolog |
| | | (S. cerevisiae)’ |
| ‘NM_024664’ | ‘PPCS’ | ‘phosphopantothenoylcysteine synthetase’ |
| ‘NM_030969’ | ‘TMEM14B’ | ‘transmembrane protein 14B’ |
| ‘NM_000288’ | ‘PEX7’ | ‘peroxisomal biogenesis factor 7’ |
| ‘NM_015975’ | ‘TAF9B’ | ‘TAF9B RNA polymerase II, TATA box binding protein |
| | | (TBP)-associated factor, 31 kDa’ |
| ‘ENST00000336420’ | ‘TAF9B’ | ‘TAF9B RNA polymerase II, TATA box binding protein |
| | | (TBP)-associated factor, 31 kDa’ |
| ‘NM_182547’ | ‘TMED4’ | ‘transmembrane emp24 protein transport domain |
| | | containing 4’ |
| ‘NM_015127’ | ‘CLCC1’ | ‘chloride channel CLIC-like 1’ |
| ‘hCT9217.2’ | ‘GTF2H5’ | ‘general transcription factor IIH, polypeptide 5’ |
| ‘NM_003071’ | ‘HLTF’ | ‘helicase-like transcription factor’ |
| ‘BC018088’ | ‘HLTF’ | ‘hypothetical protein LOC645158’ |
| ‘NM_152834’ | ‘TMEM18’ | ‘transmembrane protein 18’ |
| ‘NM_006358’ | ‘SLC25A17’ | ‘solute carrier family 25 (mitochondrial carrier; |
| | | peroxisomal membrane protein, 34 kDa), member 17’ |
| ‘NM_006358’ | ‘SLC25A17’ | ‘solute carrier family 25 (mitochondrial carrier; |
| | | peroxisomal membrane protein, 34 kDa), member 17’ |
| ‘NM_002265’ | ‘KPNB1’ | ‘karyopherin (importin) beta 1’ |
| ‘AB037853’ | ‘KIAA1432’ | ‘KIAA1432’ |
| ‘NM_017599’ | ‘VEZT’ | ‘vezatin, adherens junctions transmembrane protein’ |
| ‘NM_016312’ | ‘WBP11’ | ‘WW domain binding protein 11’ |
| ‘NM_024863’ | ‘TCEAL4’ | ‘transcription elongation factor A (SII)-like 4’ |
| ‘NM_032026’ | ‘TATDN1’ | ‘TatD DNase domain containing 1’ |
| ‘NM_003690’ | ‘PRKRA’ | ‘protein kinase, interferon-inducible double stranded RNA |
| | | dependent activator’ |
| ‘NM_020815’ | ‘PCDH10’ | ‘protocadherin 10’ |
| ‘NM_003940’ | ‘USP13’ | ‘ubiquitin specific peptidase 13 (isopeptidase T-3)’ |
| ‘Contig51621_RC’ | ‘USP13’ | ‘ubiquitin specific peptidase 13 (isopeptidase T-3)’ |
| ‘NM_018137’ | ‘PRMT6’ | ‘protein arginine methyltransferase 6’ |
| ‘NM_144981’ | ‘IMMP1L’ | ‘IMP1 inner mitochondrial membrane peptidase-like |
| | | (S. cerevisiae)’ |
| ‘NM_024592’ | ‘SRD5A3’ | ‘steroid 5 alpha-reductase 3’ |
| ‘NM_007083’ | ‘NUDT6’ | ‘nudix (nucleoside diphosphate linked moiety X)-type |
| | | motif 6’ |
| ‘NM_144597’ | ‘C15orf40’ | ‘chromosome 15 open reading frame 40’ |
| ‘HSS00211494’ | ‘C15orf40’ | ‘similar to mCG50504’ |
| ‘NM_001325’ | ‘CSTF2’ | ‘cleavage stimulation factor, 3″; pre-RNA, subunit 2, |
| | | 64 kDa’ |
| ‘NM_022909’ | ‘CENPH’ | ‘centromere protein H’ |
| ‘NM_007273’ | ‘PHB2’ | ‘prohibitin 2’ |
| ‘NM_001641’ | ‘APEX1’ | ‘APEX nuclease (multifunctional DNA repair enzyme) 1’ |
| ‘NM_080648’ | ‘APEX1’ | ‘APEX nuclease (multifunctional DNA repair enzyme) 1’ |
| ‘NM_016036’ | ‘DHRS7B’ | ‘dehydrogenase/reductase (SDR family) member 7B’ |
| ‘NM_015510’ | ‘DHRS7B’ | ‘dehydrogenase/reductase (SDR family) member 7B’ |
| ‘ENST00000297023’ | ‘SKAP2’ | ‘src kinase associated phosphoprotein 2’ |
| ‘NM_022490’ | ‘POLR1E’ | ‘polymerase (RNA) I polypeptide E, 53 kDa’ |
| ‘NM_005015’ | ‘OXA1L’ | ‘oxidase (cytochrome c) assembly 1-like’ |
| ‘NM_018066’ | ‘GPN2’ | ‘GPN-loop GTPase 2’ |
| ‘NM_181462’ | ‘MRPL55’ | ‘mitochondrial ribosomal protein L55’ |
| ‘NM_145005’ | ‘C9orf72’ | ‘chromosome 9 open reading frame 72’ |
| ‘NM_139178’ | ‘ALKBH3’ | ‘alkB, alkylation repair homolog 3 (E. coli)’ |
| ‘NM_017912’ | ‘HERC6’ | ‘hect domain and RLD 6’ |
| ‘Contig43645_RC’ | ‘CMPK2’ | ‘cytidine monophosphate (UMP-CMP) kinase 2, |
| | | mitochondrial’ |
| ‘AL079277’ | ‘PION’ | ‘pigeon homolog (Drosophila)’ |
| ‘NM_000147’ | ‘FUCA1’ | ‘fucosidase, alpha-L-1, tissue’ |
| ‘AF274932’ | ‘EIF2S3’ | ‘eukaryotic translation initiation factor 2, subunit 3 |
| | | gamma, 52 kDa’ |
| ‘NM_004403’ | ‘DFNA5’ | ‘deafness, autosomal dominant 5’ |
| ‘NM_182556’ | ‘SLC25A45’ | ‘solute carrier family 25, member 45’ |
| ‘NM_023078’ | ‘PYCRL’ | ‘pyrroline-5-carboxylate reductase-like’ |
| ‘NM_174891’ | ‘C14orf79’ | ‘chromosome 14 open reading frame 79’ |
| ‘NM_012458’ | ‘TIMM13’ | ‘translocase of inner mitochondrial membrane 13 homolog |
| | | (yeast)’ |
| ‘NM_014049’ | ‘ACAD9’ | ‘acyl-Coenzyme A dehydrogenase family, member 9’ |
| ‘NM_000178’ | ‘GSS’ | ‘glutathione synthetase’ |
| ‘NM_001610’ | ‘ACP2’ | ‘acid phosphatase 2, lysosomal’ |
| ‘NM_024887’ | ‘DHDDS’ | ‘dehydrodolichyl diphosphate synthase’ |
| ‘NM_001640’ | ‘APEH’ | ‘N-acylaminoacyl-peptide hydrolase’ |
| ‘NM_000309’ | ‘PPOX’ | ‘protoporphyrinogen oxidase’ |
| ‘NM_017967’ | ‘C19orf60’ | ‘chromosome 19 open reading frame 60’ |
| ‘NM_000447’ | ‘PSEN2’ | ‘presenilin 2 (Alzheimer disease 4)’ |
| ‘NM_031466’ | ‘TRAPPC9’ | ‘trafficking protein particle complex 9’ |
| ‘NM_022744’ | ‘C16orf58’ | ‘chromosome 16 open reading frame 58’ |
| ‘NM_001749’ | ‘CAPNS1’ | ‘calpain, small subunit 1’ |
| ‘NM_015533’ | ‘DAK’ | ‘dihydroxyacetone kinase 2 homolog (S. cerevisiae)’ |
| ‘NM_032868’ | ‘MPND’ | ‘MPN domain containing’ |
| ‘NM_032878’ | ‘ALKBH6’ | ‘alkB, alkylation repair homolog 6 (E. coli)’ |
| ‘NM_015681’ | ‘B9D1’ | ‘B9 protein domain 1’ |
| ‘ENST00000291965’ | ‘C19orf70’ | ‘chromosome 19 open reading frame 70’ |
| ‘NM_024050’ | ‘DDA1’ | ‘DET1 and DDB1 associated 1’ |
| ‘NM_006123’ | ‘IDS’ | ‘iduronate 2-sulfatase’ |
| ‘NM_020248’ | ‘CTNNBIP1’ | ‘catenin, beta interacting protein 1’ |
| ‘AB029009’ | ‘ZFR2’ | ‘zinc finger RNA binding protein 2’ |
| ‘AF039697’ | ‘NOXA1’ | ‘NADPH oxidase activator 1’ |
| ‘NM_024308’ | ‘DHRS11’ | ‘dehydrogenase/reductase (SDR family) member 11’ |
| ‘AL833240’ | ‘DHRS11’ | ‘similar to hCG2031213’ |
| ‘NM_022307’ | ‘ICA1’ | ‘islet cell autoantigen 1, 69 kDa’ |
| ‘BC028116’ | ‘ICA1’ | ‘hypothetical protein LOC730139’ |
| ‘NM_020201’ | ‘NT5M’ | ‘5″;3″;-nucleotidase, mitochondrial’ |
| ‘NM_005735’ | ‘ACTR1B’ | ‘ARP1 actin-related protein 1 homolog B, centractin beta |
| | | (yeast)’ |
| ‘NM_001001794’ | ‘FAM116B’ | ‘family with sequence similarity 116, member B’ |
| ‘AK000908’ | ‘TRIM66’ | ‘tripartite motif-containing 66’ |
| ‘Contig55446_RC’ | ‘PNPO’ | ‘pyridoxamine 5″;-phosphate oxidase’ |
| ‘NM_012272’ | ‘PRPF40B’ | ‘PRP40 pre-mRNA processing factor 40 homolog B |
| | | (S. cerevisiae)’ |
| ‘Contig38804_RC’ | ‘PRPF40B’ | ‘hypothetical LOC645460’ |
| ‘NM_032293’ | ‘GARNL3’ | ‘GTPase activating Rap/RanGAP domain-like 3’ |
| ‘NM_015512’ | ‘DNAH1’ | ‘dynein, axonemal, heavy chain 1’ |
| ‘U79260’ | ‘FTO’ | ‘fat mass and obesity associated’ |
| ‘NM_000727’ | ‘CACNG1’ | ‘calcium channel, voltage-dependent, gamma subunit 1’ |
| ‘NM_152361’ | ‘EID2B’ | ‘EP300 interacting inhibitor of differentiation 2B’ |
| ‘NM_005342’ | ‘HMGB3’ | ‘high-mobility group box 3’ |
| ‘NM_024109’ | ‘C16orf68’ | ‘chromosome 16 open reading frame 68’ |
| ‘NM_001139’ | ‘ALOX12B’ | ‘arachidonate 12-lipoxygenase, 12R type’ |
| ‘NM_000250’ | ‘MPO’ | ‘myeloperoxidase’ |
| ‘NM_153274’ | ‘BEST4’ | ‘bestrophin 4’ |
| ‘NM_152497’ | ‘STMN1’ | ‘stathmin 1’ |
| ‘AF339771’ | ‘STMN1’ | ‘hypothetical LOC100129122’ |
| ‘NM_153248’ | ‘STMN1’ | ‘Hypothetical protein LOC653160’ |
| ‘NM_012391’ | ‘SPDEF’ | ‘SAM pointed domain containing ets transcription factor’ |
| ‘Contig35292_RC’ | ‘FAM66D’ | ‘family with sequence similarity 66, member D’ |
| ‘NM_032653’ | ‘C21orf122’ | ‘chromosome 21 open reading frame 122’ |
| ‘NM_005783’ | ‘TXNDC9’ | ‘thioredoxin domain containing 9’ |
| ‘NM_024903’ | ‘ZNF721’ | ‘zinc finger protein 721’ |
| ‘hCT1820084.2’ | ‘LIPJ’ | ‘lipase, family member J’ |
| ‘BC040303’ | ‘LIPJ’ | ‘hypothetical protein LOC727916’ |
| ‘NM_173831’ | ‘ZNF707’ | ‘zinc finger protein 707’ |
| ‘NM_014592’ | ‘KCNIP1’ | ‘Kv channel interacting protein 1’ |
| ‘NM_014264’ | ‘PLK4’ | ‘polo-like kinase 4 (Drosophila)’ |
| ‘ENST00000298789’ | ‘ENO4’ | ‘enolase family member 4’ |
| ‘HSS00014253’ | ‘ENO4’ | ‘hypothetical LOC151760’ |
| ‘AL133568’ | ‘ENO4’ | ‘hypothetical protein LOC613126’ |
| ‘BC035660’ | ‘TMSB15B’ | ‘thymosin beta 15B’ |
| ‘Contig23804_RC’ | ‘TMSB15B’ | ‘hypothetical LOC100129282’ |
| ‘Contig37577_RC’ | ‘TMSB15B’ | ‘hypothetical LOC643783’ |
| ‘NM_001082’ | ‘CYP4F2’ | ‘cytochrome P450, family 4, subfamily F, polypeptide 2’ |
| ‘Contig49652_RC’ | ‘CEP78’ | ‘centrosomal protein 78 kDa’ |
| ‘NM_002012’ | ‘FHIT’ | ‘fragile histidine triad gene’ |
| ‘NM_006491’ | ‘NOVA1’ | ‘neuro-oncological ventral antigen 1’ |
| ‘AK090949’ | ‘NOVA1’ | ‘hypothetical LOC644873’ |
| ‘NM_004038’ | ‘AMY1A’ | ‘amylase, alpha 1A (salivary)’ |
| ‘NM_020978’ | ‘AMY2B’ | ‘amylase, alpha 2B (pancreatic)’ |
| ‘NM_020121’ | ‘UGGT2’ | ‘UDP-glucose glycoprotein glucosyltransferase 2’ |
| ‘NM_016008’ | ‘DYNC2LI1’ | ‘dynein, cytoplasmic 2, light intermediate chain 1’ |
| ‘BC015894’ | ‘MTR’ | ‘5-methyltetrahydrofolate-homocysteine |
| | | methyltransferase’ |
| ‘NM_003304’ | ‘TRPC1’ | ‘transient receptor potential cation channel, subfamily C, |
| | | member 1’ |
| ‘NM_021931’ | ‘DHX35’ | ‘DEAH (Asp-Glu-Ala-His) box polypeptide 35’ |
| ‘NM_173622’ | ‘CDRT4’ | ‘CMT1A duplicated region transcript 4’ |
| ‘AK058162’ | ‘PGPEP1L’ | ‘pyroglutamyl-peptidase I-like’ |
| ‘HSS00298733’ | ‘PYCR2’ | ‘pyrroline-5-carboxylate reductase family, member 2’ |
| ‘NM_144620’ | ‘LRRC39’ | ‘leucine rich repeat containing 39’ |
| ‘NM_000535’ | ‘PMS2’ | ‘PMS2 postmeiotic segregation increased 2 (S. cerevisiae)’ |
| ‘Contig31296_RC’ | ‘PMS2’ | ‘hypothetical protein FLJ10038’ |
| ‘NM_145858’ | ‘CRYZL1’ | ‘crystallin, zeta (quinone reductase)-like 1’ |
| ‘NM_018040’ | ‘GPATCH2’ | ‘G patch domain containing 2’ |
| ‘NM_033317’ | ‘DMKN’ | ‘dermokine’ |
| ‘NM_024687’ | ‘ZBBX’ | ‘zinc finger, B-box domain containing’ |
| ‘BC040874’ | ‘ZNF518B’ | ‘zinc finger protein 518B’ |
| ‘NM_032202’ | ‘KIAA1109’ | ‘KIAA1109’ |
| ‘AK054953’ | ‘KIAA1109’ | ‘hypothetical protein LOC200830’ |
| ‘D38437’ | ‘PMS2L3’ | ‘postmeiotic segregation increased 2-like 3’ |
| ‘NM_005395’ | ‘PMS2L3’ | ‘postmeiotic segregation increased 2-like 3’ |
| ‘NM_003019’ | ‘SFTPD’ | ‘surfactant protein D’ |
| ‘NM_004192’ | ‘ASMTL’ | ‘acetylserotonin O-methyltransferase-like’ |
| ‘NM_058163’ | ‘TSR2’ | ‘TSR2, 20S rRNA accumulation, homolog (S. cerevisiae)’ |
| ‘NM_022078’ | ‘GPATCH3’ | ‘G patch domain containing 3’ |
| ‘NM_139015’ | ‘UNQ1887’ | ‘signal peptide peptidase 3’ |
| ‘NM_181493’ | ‘ITPA’ | ‘inosine triphosphatase (nucleoside triphosphate |
| | | pyrophosphatase)’ |
| ‘Contig20708_RC’ | ‘RCOR3’ | ‘REST corepressor 3’ |
| ‘ENST00000295647’ | ‘RCOR3’ | ‘hypothetical LOC645676’ |
| ‘AL713756’ | ‘RCOR3’ | ‘hypothetical LOC202781’ |
| ‘NM_001513’ | ‘GSTZ1’ | ‘glutathione transferase zeta 1’ |
| ‘NM_145871’ | ‘GSTZ1’ | ‘glutathione transferase zeta 1’ |
| ‘NM_014234’ | ‘HSD17B8’ | ‘hydroxysteroid (17-beta) dehydrogenase 8’ |
| ‘Contig49181_RC’ | ‘C9orf103’ | ‘chromosome 9 open reading frame 103’ |
| ‘NM_001609’ | ‘ACADSB’ | ‘acyl-Coenzyme A dehydrogenase, short/branched chain’ |
| ‘XM_210879’ | ‘ACADSB’ | ‘hypothetical LOC100128511’ |
| ‘NM_018622’ | ‘PARL’ | ‘presenilin associated, rhomboid-like’ |
| ‘NM_001280’ | ‘CIRBP’ | ‘cold inducible RNA binding protein’ |
| ‘NM_006743’ | ‘RBM3’ | ‘RNA binding motif (RNP1, RRM) protein 3’ |
| ‘NM_006304’ | ‘SHFM1’ | ‘split hand/foot malformation (ectrodactyly) type 1’ |
| ‘NM_012176’ | ‘FBXO4’ | ‘F-box protein 4’ |
| ‘Contig51015_RC’ | ‘FBXO4’ | ‘similar to hCG1811779’ |
| ‘NM_015919’ | ‘ZNF226’ | ‘zinc finger protein 226’ |
| ‘HSS00124019’ | ‘HNRNPA1L2’ | ‘heterogeneous nuclear ribonucleoprotein A1-like 2’ |
| ‘NM_178324’ | ‘SPTLC1’ | ‘serine palmitoyltransferase, long chain base subunit 1’ |
| ‘NM_173554’ | ‘C10orf107’ | ‘chromosome 10 open reading frame 107’ |
| ‘Contig48954_RC’ | ‘C10orf107’ | ‘hypothetical LOC400099’ |
| ‘AF086472’ | ‘C10orf107’ | ‘hypothetical protein LOC728769’ |
| ‘NM_080662’ | ‘PEX11G’ | ‘peroxisomal biogenesis factor 11 gamma’ |
| ‘NM_024108’ | ‘TRAPPC6A’ | ‘trafficking protein particle complex 6A’ |
| ‘NM_006584’ | ‘CCT6B’ | ‘chaperonin containing TCP1, subunit 6B (zeta 2)’ |
| ‘Contig50013_RC’ | ‘ZNF18’ | ‘zinc finger protein 18’ |
| ‘NM_018696’ | ‘ELAC1’ | ‘elaC homolog 1 (E. coli)’ |
| ‘NM_020677’ | ‘NMRAL1’ | ‘NmrA-like family domain containing 1’ |
| ‘NM_004813’ | ‘PEX16’ | ‘peroxisomal biogenesis factor 16’ |
| ‘NM_002582’ | ‘PARN’ | ‘poly(A)-specific ribonuclease (deadenylation nuclease)’ |
| ‘AK023312’ | ‘hCG_2022304’ | ‘similar to hCG2022304’ |
| ‘hCT1970806.1’ | ‘hCG_2022304’ | ‘embigin homolog (mouse) pseudogene’ |
| ‘Contig40887_RC’ | ‘hCG_2022304’ | ‘hypothetical protein LOC153546’ |
| ‘AK091261’ | ‘METT5D1’ | ‘methyltransferase 5 domain containing 1’ |
| ‘AK096857’ | ‘METT5D1’ | ‘hypothetical LOC646999’ |
| ‘NM_024061’ | ‘ZNF655’ | ‘zinc finger protein 655’ |
| ‘Contig51068_RC’ | ‘ZNF655’ | ‘hypothetical LOC100128822’ |
| ‘NM_024642’ | ‘GALNT12’ | ‘UDP-N-acetyl-alpha-D-galactosamine: polypeptide N- |
| | | acetylgalactosaminyltransferase 12 (GalNAc-T12)’ |
| ‘NM_017703’ | ‘FBXL12’ | T-box and leucine-rich repeat protein 12’ |
| ‘NM_000254’ | ‘MTR’ | ‘5-methyltetrahydrofolate-homocysteine |
| | | methyltransferase’ |
| ‘NM_024648’ | ‘C17orf101’ | ‘chromosome 17 open reading frame 101’ |
| ‘NM_052861’ | ‘C4orf42’ | ‘chromosome 4 open reading frame 42’ |
| ‘NM_032712’ | ‘C19orf48’ | ‘chromosome 19 open reading frame 48’ |
| ‘NM_032309’ | ‘CHCHD5’ | ‘coiled-coil-helix-coiled-coil-helix domain containing 5’ |
| ‘NM_032705’ | ‘C1orf97’ | ‘chromosome 1 open reading frame 97’ |
| ‘NM_003865’ | ‘HESX1’ | ‘HESX homeobox 1’ |
| ‘NM_016028’ | ‘SUV420H1’ | ‘suppressor of variegation 4-20 homolog 1 (Drosophila)’ |
| ‘NM_175085’ | ‘GART’ | ‘phosphoribosylglycinamide formyltransferase, |
| | | phosphoribosylglycinamide synthetase, |
| | | phosphoribosylaminoimidazole synthetase’ |
| ‘BC019888’ | ‘ZKSCAN3’ | ‘zinc finger with KRAB and SCAN domains 3’ |
| ‘NM_014641’ | ‘MDC1’ | ‘mediator of DNA-damage checkpoint 1’ |
| ‘NM_006110’ | ‘CD2BP2’ | ‘CD2 (cytoplasmic tail) binding protein 2’ |
| ‘NM_014346’ | ‘TBC1D22A’ | ‘TBC1 domain family, member 22A’ |
| ‘NM_000048’ | ‘ASL’ | ‘argininosuccinate lyase’ |
| ‘NM_007022’ | ‘CYB561D2’ | ‘cytochrome b-561 domain containing 2’ |
| ‘NM_014908’ | ‘DOLK’ | ‘dolichol kinase’ |
| ‘NM_006066’ | ‘AKR1A1’ | ‘aldo-keto reductase family 1, member A1 (aldehyde |
| | | reductase)’ |
| ‘NM_153326’ | ‘AKR1A1’ | ‘aldo-keto reductase family 1, member A1 (aldehyde |
| | | reductase)’ |
| ‘Contig24161_RC’ | ‘ZDHHC24’ | ‘zinc finger, DHHC-type containing 24’ |
| ‘NM_024805’ | ‘C18orf22’ | ‘chromosome 18 open reading frame 22’ |
| ‘NM_015411’ | ‘SUMF2’ | ‘sulfatase modifying factor 2’ |
| ‘NM_032815’ | ‘NFATC2IP’ | ‘nuclear factor of activated T-cells, cytoplasmic, |
| | | calcineurin-dependent 2 interacting protein’ |
| ‘NM_138436’ | ‘C8orf40’ | ‘chromosome 8 open reading frame 40’ |
| ‘NM_144611’ | ‘CYB5D2’ | ‘cytochrome b5 domain containing 2’ |
| ‘NM_006443’ | ‘C6orf108’ | ‘chromosome 6 open reading frame 108’ |
| ‘Contig6323_RC’ | ‘NANOG’ | ‘Nanog homeobox’ |
| ‘NM_024865’ | ‘NANOG’ | ‘Nanog homeobox’ |
| ‘NM_022129’ | ‘PBLD’ | ‘phenazine biosynthesis-like protein domain containing’ |
| ‘NM_020817’ | ‘KIAA1407’ | ‘KIAA1407’ |
| ‘NM_018079’ | ‘SRBD1’ | ‘S1 RNA binding domain 1’ |
| ‘NM_001334’ | ‘CTSO’ | ‘cathepsin O’ |
| ‘NM_176815’ | ‘DHFRL1’ | ‘dihydrofolate reductase-like 1’ |
| ‘NM_017807’ | ‘OSGEP’ | ‘O-sialoglycoprotein endopeptidase’ |
| ‘NM_001333’ | ‘CTSL2’ | ‘cathepsin L2’ |
| ‘Contig39301_RC’ | ‘TTLL11’ | ‘tubulin tyrosine ligase-like family, member 11’ |
| ‘NM_005276’ | ‘GPD1’ | ‘glycerol-3-phosphate dehydrogenase 1 (soluble)’ |
| ‘NM_152402’ | TRAM1L1’ | ‘translocation associated membrane protein 1-like 1’ |
| ‘NM_033031’ | ‘CCNB3’ | ‘cyclin B3’ |
| ‘Contig56583_RC’ | ‘SPAG16’ | ‘sperm associated antigen 16’ |
| ‘NM_014683’ | ‘ULK2’ | ‘unc-51-like kinase 2 (C. elegans)’ |
| ‘NM_000140’ | ‘FECH’ | ‘ferrochelatase (protoporphyria)’ |
| ‘NM_014924’ | ‘KIAA0831’ | ‘KIAA0831’ |
| ‘NM_014733’ | ‘ZFYVE16’ | ‘zinc finger, FYVE domain containing 16’ |
| ‘NM_014477’ | ‘TP53TG5’ | ‘TP53 target 5’ |
| ‘NM_013356’ | ‘SLC16A8’ | ‘solute carrier family 16, member 8 (monocarboxylic acid |
| | | transporter 3)’ |
| ‘NM_013356’ | ‘SLC16A8’ | ‘solute carrier family 16, member 8 (monocarboxylic acid |
| | | transporter 3)’ |
| ‘NM_016444’ | ‘ZNF226’ | ‘zinc finger protein 226’ |
| ‘NM_033207’ | ‘OPALIN’ | ‘oligodendrocytic myelin paranodal and inner loop protein’ |
| ‘NM_013328’ | ‘PYCR2’ | ‘pyrroline-5-carboxylate reductase family, member 2’ |
| ‘NM_178564’ | ‘NRBP2’ | ‘nuclear receptor binding protein 2’ |
| ‘NM_031483’ | ‘ITCH’ | ‘itchy E3 ubiquitin protein ligase homolog (mouse)’ |
| ‘AK001223’ | ‘ITCH’ | ‘anaphase promoting complex subunit 1 pseudogene’ |
| ‘NM_012070’ | ‘ATRN’ | ‘attractin’ |
| ‘NM_139322’ | ‘ATRN’ | ‘attractin’ |
| ‘Contig29513_RC’ | ‘ATRN’ | ‘hypothetical protein LOC100129722’ |
| ‘AK056063’ | ‘ATRN’ | ‘hypothetical protein LOC100128788’ |
| ‘NM_002477’ | ‘MYL5’ | ‘myosin, light chain 5, regulatory’ |
| ‘NM_173542’ | ‘PLBD2’ | ‘phospholipase B domain containing 2’ |
| ‘XM_212067’ | ‘C7orf13’ | ‘chromosome 7 open reading frame 13’ |
| ‘NM_031948’ | ‘PRSS27’ | ‘protease, serine 27’ |
| ‘AK055800’ | ‘C17orf49’ | ‘chromosome 17 open reading frame 49’ |
| ‘NM_014042’ | ‘C11orf51’ | ‘chromosome 11 open reading frame 51’ |
| ‘NM_182572’ | ‘ZSCAN1’ | ‘zinc finger and SCAN domain containing 1’ |
| ‘NM_198061’ | ‘CES2’ | ‘carboxylesterase 2 (intestine, liver)’ |
| ‘NM_012191’ | ‘NAT6’ | ‘N-acetyltransferase 6 (GCN5-related)’ |
| ‘AK095567’ | ‘NAT6’ | ‘hypothetical protein LOC284014’ |
| ‘NM_023924’ | ‘BRD9’ | ‘bromodomain containing 9’ |
| ‘NM_016154’ | ‘RAB4B’ | ‘RAB4B, member RAS oncogene family’ |
| ‘NM_007230’ | ‘MAN1B1’ | ‘mannosidase, alpha, class 1B, member 1’ |
| ‘NM_016219’ | ‘MAN1B1’ | ‘mannosidase, alpha, class 1B, member 1’ |
| ‘NM_153335’ | ‘STRADA’ | ‘STE20-related kinase adaptor alpha’ |
| ‘NM_019625’ | ‘ABCB9’ | ‘ATP-binding cassette, sub-family B (MDR/TAP), |
| | | member 9’ |
| ‘NM_203444’ | ‘ABCB9’ | ‘ATP-binding cassette, sub-family B (MDR/TAP), |
| | | member 9’ |
| ‘AB058765’ | ‘KRBA1’ | ‘KRAB-A domain containing 1’ |
| ‘NM_017438’ | ‘SETD4’ | ‘SET domain containing 4’ |
| ‘ENST00000282333’ | ‘ZNF837’ | ‘zinc finger protein 837’ |
| ‘NM_024591’ | ‘CHMP6’ | ‘chromatin modifying protein 6’ |
| ‘NM_012163’ | ‘LRRC29’ | ‘leucine rich repeat containing 29’ |
| ‘NM_017999’ | ‘RNF31’ | ‘ring finger protein 31’ |
| ‘NM_025161’ | ‘C17orf70’ | ‘chromosome 17 open reading frame 70’ |
| ‘NM_016035’ | ‘COQ4’ | ‘coenzyme Q4 homolog (S. cerevisiae)’ |
| ‘NM_138355’ | ‘SCRN2’ | ‘secernin 2’ |
| ‘NM_182480’ | ‘COQ6’ | ‘coenzyme Q6 homolog, monooxygenase (S. cerevisiae)’ |
| ‘NM_139242’ | ‘MTFMT’ | ‘mitochondrial methionyl-tRNA |
| | | formyltransferase’ |
| ‘NM_014844’ | ‘TECPR2’ | ‘tectonin beta-propeller repeat containing 2’ |
| ‘NM_024705’ | ‘DHRS12’ | ‘dehydrogenase/reductase (SDR family) member 12’ |
| ‘NM_004957’ | ‘FPCS’ | ‘folylpolyglutamate synthase’ |
| ‘NM_000199’ | ‘SGSH’ | ‘N-sulfoglucosamine sulfohydrolase’ |
| ‘NM_022773’ | ‘LMF1’ | ‘lipase maturation factor 1’ |
| ‘NM_006453’ | ‘TBL3’ | ‘transducin (beta)-like 3’ |
| ‘NM_016602’ | ‘CCR10’ | ‘chemokine (C-C motif) receptor 10’ |
| ‘NM_000403’ | ‘GALE’ | ‘UDP-galactose-4-epimerase’ |
| ‘NM_014413’ | ‘EIF2AK1’ | ‘eukaryotic translation initiation factor 2-alpha kinase 1’ |
| ‘NM_004043’ | ‘ASMT’ | ‘acetylserotonin O-methyltransferase’ |
| ‘NM_032292’ | ‘GON4L’ | ‘gon-4-like (C. elegans)’ |
| ‘NM_004895’ | ‘NLRP3’ | ‘NLR family, pyrin domain containing 3’ |
| ‘NM_014674’ | ‘EDEM1’ | ‘ER degradation enhancer, mannosidase alpha-like 1’ |
| ‘NM_032837’ | ‘FAM104A’ | ‘family with sequence similarity 104, member A’ |
| ‘NM_152647’ | ‘C15orf33’ | ‘chromosome 15 open reading frame 33’ |
| ‘NM_002899’ | ‘RBP1’ | ‘retinol binding protein 1, cellular’ |
| ‘NM_025188’ | ‘TRIM45’ | ‘tripartite motif-containing 45’ |
| ‘NM_148172’ | ‘PEMT’ | ‘phosphatidylethanolamine N-methyltransferase’ |
| ‘Contig57441_RC’ | ‘PEMT’ | ‘similar to hCG1806822’ |
| ‘NM_032350’ | ‘C7orf50’ | ‘chromosome 7 open reading frame 50’ |
| ‘NM_013274’ | ‘POLL’ | ‘polymerase (DNA directed), lambda’ |
| ‘NM_145241’ | ‘WDR31’ | ‘WD repeat domain 31’ |
| ‘NM_004914’ | ‘RAB36’ | ‘RAB36, member RAS oncogene family’ |
| ‘AL096749’ | ‘C1orf175’ | ‘chromosome 1 open reading frame 175’ |
| ‘NM_018116’ | ‘MSTO1’ | ‘misato homolog 1 (Drosophila)’ |
| ‘NM_003273’ | ‘TM7SF2’ | ‘transmembrane 7 superfamily member 2’ |
| ‘NM_006051’ | ‘APBB3’ | ‘amyloid beta (A4) precursor protein-binding, family B, |
| | | member 3’ |
| ‘NM_021210’ | ‘TRAPPC1’ | ‘trafficking protein particle complex 1’ |
| ‘NM_033416’ | ‘IMP4’ | ‘IMP4, U3 small nucleolar ribonucleoprotein, homolog |
| | | (yeast)’ |
| ‘NM_005600’ | ‘NIT1’ | ‘nitrilase 1’ |
| ‘NM_005881’ | ‘BCKDK’ | ‘branched chain ketoacid dehydrogenase kinase’ |
| ‘Contig51986_RC’ | ‘PTRH1’ | ‘peptidyl-tRNA hydrolase 1 homolog (S. cerevisiae)’ |
| ‘NM_024084’ | ‘TMEM223’ | ‘transmembrane protein 223’ |
| ‘NM_144564’ | ‘SLC39A3’ | ‘solute carrier family 39 (zinc transporter), member 3’ |
| ‘NM_032928’ | ‘TMEM141’ | ‘transmembrane protein 141’ |
| ‘NM_198527’ | ‘HDDC3’ | ‘HD domain containing 3’ |
| ‘NM_148914’ | ‘ABHD11’ | ‘abhydrolase domain containing 11’ |
| ‘NM_031295’ | ‘ABHD11’ | ‘abhydrolase domain containing 11’ |
| ‘NM_015944’ | ‘AMDHD2’ | ‘amidohydrolase domain containing 2’ |
| ‘NM_013321’ | ‘SNX8’ | ‘sorting nexin 8’ |
| ‘NM_006396’ | ‘SSSCA1’ | ‘Sjogren syndrome/scleroderma autoantigen 1’ |
| ‘NM_024662’ | ‘NAT10’ | ‘N-acetyltransferase 10 (GCN5-related)’ |
| ‘NM_022719’ | ‘DGCR14’ | ‘DiGeorge syndrome critical region gene 14’ |
| ‘NM_138350’ | ‘THAP3’ | ‘THAP domain containing, apoptosis associated protein 3’ |
| ‘NM_001384’ | ‘DPH2’ | ‘DPH2 homolog (S. cerevisiae)’ |
| ‘NM_024587’ | ‘TMEM53’ | ‘transmembrane protein 53’ |
| ‘Contig39875’ | ‘CDNF’ | ‘cerebral dopamine neurotrophic factor’ |
| ‘hCT2319126’ | ‘CCDC14’ | ‘coiled-coil domain containing 14’ |
| ‘HSS00051366’ | ‘FABP5’ | ‘fatty acid binding protein 5 (psoriasis-associated)’ |
| ‘NM_022128’ | ‘RBKS’ | ‘ribokinase’ |
| ‘NM_147172’ | ‘NUDT2’ | ‘nudix (nucleoside diphosphate linked moiety X)-type |
| | | motif 2’ |
| ‘NM_006032’ | ‘CPNE6’ | ‘copine VI (neuronal)’ |
| ‘NM_004650’ | ‘PNPLA4’ | ‘patatin-like phospholipase domain containing 4’ |
| ‘NM_144967’ | ‘RP13-102H20.1’ | ‘hypothetical protein FLJ30058’ |
| ‘NM_032561’ | ‘C22orf23’ | ‘chromosome 22 open reading frame 23’ |
| ‘NM_033028’ | ‘BBS4’ | ‘Bardet-Biedl syndrome 4’ |
| ‘NM_130810’ | ‘DYX1C1’ | ‘dyslexia susceptibility 1 candidate 1’ |
| ‘NM_004855’ | ‘PIGB’ | ‘phosphatidylinositol glycan anchor biosynthesis, class B’ |
| ‘AK058070’ | ‘MDH1B’ | ‘malate dehydrogenase 1B, NAD (soluble)’ |
| ‘ENST00000282535’ | ‘ZCWPW2’ | ‘zinc finger, CW type with PWWP domain 2’ |
| ‘AF277187’ | ‘PTPMT1’ | ‘protein tyrosine phosphatase, mitochondrial 1’ |

TABLE 7
Correlated Genes for Alz

| RefSeq Gene | | |
|---|---|---|
| Transcript | Gene | |
| Identification | Symbol | Gene Name/Description |
| ‘NM_000961’ | ‘PTGIS’ | ‘prostaglandin I2 (prostacyclin) synthase’ |
| ‘NM_178275’ | ‘IGFN1’ | ‘immunoglobulin-like and fibronectin type III domain |
| | | containing 1’ |
| ‘NM_031911’ | ‘C1QTNF7’ | ‘C1q and tumor necrosis factor related protein 7’ |
| ‘NM_053056’ | ‘CCND1’ | ‘cyclin D1’ |
| ‘NM_003278’ | ‘CLEC3B’ | ‘C-type lectin domain family 3, member B’ |
| ‘NM_003271’ | ‘TSPAN4’ | ‘tetraspanin 4’ |
| ‘NM_170696’ | ‘ALDH1A2’ | ‘aldehyde dehydrogenase 1 family, member A2’ |
| ‘NM_178822’ | ‘IGSF10’ | ‘immunoglobulin superfamily, member 10’ |
| ‘NM_024574’ | ‘C4orf31’ | ‘chromosome 4 open reading frame 31’ |
| ‘Contig15600_RC’ | ‘SLC9A2’ | ‘solute carrier family 9 (sodium/hydrogen exchanger), |
| | | member 2’ |
| ‘NM_020190’ | ‘OLFML3’ | ‘olfactomedin-like 3’ |
| ‘NM_004484’ | ‘GPC3’ | ‘glypican 3’ |
| ‘AK093936’ | ‘GPC3’ | ‘hypothetical LOC284276’ |
| ‘NM_003226’ | ‘TFF3’ | ‘trefoil factor 3 (intestinal)’ |
| ‘NM_017459’ | ‘MFAP2’ | ‘microfibrillar-associated protein 2’ |
| ‘NM_031935’ | ‘HMCN1’ | ‘hemicentin 1’ |
| ‘Contig36517_RC’ | ‘PDE5A’ | ‘phosphodiesterase 5A, cGMP-specific’ |
| ‘Contig56611_RC’ | ‘PDE5A’ | ‘phosphodiesterase 5A, cGMP-specific’ |
| ‘NM_005460’ | ‘SNCAIP’ | ‘synuclein, alpha interacting protein’ |
| ‘NM_205855’ | ‘FAM180A’ | ‘family with sequence similarity 180, member A’ |
| ‘NM_002178’ | ‘IGFBP6’ | ‘insulin-like growth factor binding protein 6’ |
| ‘NM_153226’ | ‘TMEM20’ | ‘transmembrane protein 20’ |
| ‘NM_025208’ | ‘PDGFD’ | ‘platelet derived growth factor D’ |
| ‘NM_001878’ | ‘CRABP2’ | ‘cellular retinoic acid binding protein 2’ |
| ‘NM_006034’ | ‘TP53I11’ | ‘tumor protein p53 inducible protein 11’ |
| ‘NM_021977’ | ‘SLC22A3’ | ‘solute carrier family 22 (extraneuronal monoamine |
| | | transporter), member 3’ |
| ‘NM_000597_sat’ | ‘IGFBP2’ | ‘insulin-like growth factor binding protein 2, 36 kDa’ |
| ‘NM_000597’ | ‘IGFBP2’ | ‘insulin-like growth factor binding protein 2, 36 kDa’ |
| ‘NM_130851’ | ‘BMP4’ | ‘bone morphogenetic protein 4’ |
| ‘NM_002216_sat’ | ‘ITIH2’ | ‘inter-alpha (globulin) inhibitor H2’ |
| ‘NM_002216’ | ‘ITIH2’ | ‘inter-alpha (globulin) inhibitor H2’ |
| ‘NM_002216’ | ‘ITIH2’ | ‘inter-alpha (globulin) inhibitor H2’ |
| ‘NM_032411’ | ‘C2orf40’ | ‘chromosome 2 open reading frame 40’ |
| ‘Contig53033_RC’ | ‘CPXM2’ | ‘carboxypeptidase X (M14 family), member 2’ |
| ‘NM_007366’ | ‘PLA2R1’ | ‘phospholipase A2 receptor 1, 180 kDa’ |
| ‘NM_138299’ | ‘MUC4’ | ‘mucin 4, cell surface associated’ |
| ‘NM_052832’ | ‘SLC26A7’ | ‘solute carrier family 26, member 7’ |
| ‘NM_020639’ | ‘RIPK4’ | ‘receptor-interacting serine-threonine kinase 4’ |
| ‘NM_022369’ | ‘STRA6’ | ‘stimulated by retinoic acid gene 6 homolog (mouse)’ |
| ‘AL080078’ | ‘TMEM30B’ | ‘transmembrane protein 30B’ |
| ‘NM_145753’ | ‘PHLDB2’ | ‘pleckstrin homology-like domain, family B, member 2’ |
| ‘NM_000474’ | ‘TWIST1’ | ‘twist homolog 1 (Drosophila)’ |
| ‘NM_021219’ | ‘JAM2’ | ‘junctional adhesion molecule 2’ |
| ‘NM_000777’ | ‘CYP3A5’ | ‘cytochrome P450, family 3, subfamily A, polypeptide 5’ |
| ‘AY582531’ | ‘CYP3A5’ | ‘cytochrome P450 3A64’ |
| ‘AY334551’ | ‘CYP3A5’ | ‘cytochrome P450 3A64’ |
| ‘NM_032387’ | ‘WNK4’ | ‘WNK lysine deficient protein kinase 4’ |
| ‘NM_178817’ | ‘MRAP’ | ‘melanocortin 2 receptor accessory protein’ |
| ‘NM_002048’ | ‘GAS1’ | ‘growth arrest-specific 1’ |
| ‘NM_002303’ | ‘LEPR’ | ‘leptin receptor’ |
| ‘Contig47453_RC’ | ‘AFAP1L1’ | ‘actin filament associated protein 1-like 1’ |
| ‘NM_005218’ | ‘DEFB1’ | ‘defensin, beta 1’ |
| ‘NM_016412’ | ‘IGF2AS’ | ‘insulin-like growth factor 2 antisense’ |
| ‘NM_021977’ | ‘SLC22A3’ | ‘solute carrier family 22 (extraneuronal monoamine |
| | | transporter), member 3’ |
| ‘NM_024605’ | ‘ARHGAP10’ | ‘Rho GTPase activating protein 10’ |
| ‘NM_052858’ | ‘MARVELD3’ | ‘MARVEL domain containing 3’ |
| ‘AB041269’ | ‘KRT19P2’ | ‘keratin 19 pseudogene 2’ |
| ‘NM_002276’ | ‘KRT19’ | ‘keratin 19’ |
| ‘NM_019609’ | ‘CPXM1’ | ‘carboxypeptidase X (M14 family), member 1’ |
| ‘HSS00141347’ | ‘CPXM1’ | ‘hypothetical LOC339535’ |
| ‘NM_007361’ | ‘NID2’ | ‘nidogen 2 (osteonidogen)’ |
| ‘NM_006039’ | ‘MRC2’ | ‘mannose receptor, C type 2’ |
| ‘NM_000959’ | ‘PTGFR’ | ‘prostaglandin F receptor (FP)’ |
| ‘NM_000396’ | ‘CTSK’ | ‘cathepsin K’ |
| ‘AK026784’ | ‘ITGBL1’ | ‘integrin, beta-like 1 (with EGF-like repeat domains)’ |
| ‘NM_004791’ | ‘ITGBL1’ | ‘integrin, beta-like 1 (with EGF-like repeat domains)’ |
| ‘NM_024423’ | ‘DSC3’ | ‘desmocollin 3’ |
| ‘Contig48945_RC’ | ‘DSG2’ | ‘desmoglein 2’ |
| ‘NM_001943’ | ‘DSG2’ | ‘desmoglein 2’ |
| ‘NM_004572’ | ‘PKP2’ | ‘plakophilin 2’ |
| ‘NM_031200’ | ‘CCR9’ | ‘chemokine (C-C motif) receptor 9’ |
| ‘NM_153279’ | ‘SLC22A6’ | ‘solute carrier family 22 (organic anion transporter), |
| | | member 6’ |
| ‘NM_004790’ | ‘SLC22A6’ | ‘solute carrier family 22 (organic anion transporter), |
| | | member 6’ |
| ‘NM_004790’ | ‘SLC22A6’ | ‘solute carrier family 22 (organic anion transporter), |
| | | member 6’ |
| ‘Contig16712_RC’ | ‘SMTNL2’ | ‘smoothelin-like 2’ |
| ‘NM_004254’ | ‘SLC22A8’ | ‘solute carrier family 22 (organic anion transporter), |
| | | member 8’ |
| ‘Contig33444_RC’ | ‘MARVELD3’ | ‘MARVEL domain containing 3’ |
| ‘NM_001266’ | ‘CES1’ | ‘carboxylesterase 1 (monocyte/macrophage serine esterase 1)’ |
| ‘NM_001266’ | ‘CES1’ | ‘carboxylesterase 1 (monocyte/macrophage serine esterase 1)’ |
| ‘NM_001078’ | ‘VCAM1’ | ‘vascular cell adhesion molecule 1’ |
| ‘XM_113636’ | ‘SLC16A12’ | ‘solute carrier family 16, member 12 (monocarboxylic acid |
| | | transporter 12)’ |
| ‘NM_000088’ | ‘COL1A1’ | ‘collagen, type I, alpha 1’ |
| ‘NM_004835’ | ‘AGTR1’ | ‘angiotensin II receptor, type 1’ |
| ‘NM_000685’ | ‘AGTR1’ | ‘angiotensin II receptor, type 1’ |
| ‘NM_006329’ | ‘FBLN5’ | ‘fibulin 5’ |
| ‘NM_021073’ | ‘BMP5’ | ‘bone morphogenetic protein 5’ |
| ‘NM_000953’ | ‘PTGDR’ | ‘prostaglandin D2 receptor (DP)’ |
| ‘NM_018242’ | ‘SLC47A1’ | ‘solute carrier family 47, member 1’ |
| ‘Contig29982_RC’ | ‘SCARA5’ | ‘scavenger receptor class A, member 5 (putative)’ |
| ‘NM_016307’ | ‘PRRX2’ | ‘paired related homeobox 2’ |
| ‘NM_003064’ | ‘SLPI’ | ‘secretory leukocyte peptidase inhibitor’ |
| ‘NM_003066’ | ‘SLPI’ | ‘secretory leukocyte peptidase inhibitor’ |
| ‘NM_001463’ | ‘FRZB’ | ‘frizzled-related protein’ |
| ‘AF318382’ | ‘IGF2’ | ‘insulin-like growth factor 2 (somatomedin A)’ |
| ‘NM_003652’ | ‘CPZ’ | ‘carboxypeptidase Z’ |
| ‘NM_000504’ | ‘F10’ | ‘coagulation factor X’ |
| ‘NM_002253’ | ‘KDR’ | ‘kinase insert domain receptor (a type III receptor tyrosine |
| | | kinase)’ |
| ‘NM_004369’ | ‘COL6A3’ | ‘collagen, type VI, alpha 3’ |
| ‘NM_002023’ | ‘FMOD’ | ‘fibromodulin’ |
| ‘AB033025’ | ‘KIAA1199’ | ‘KIAA1199’ |
| ‘NM_145260’ | ‘OSR1’ | ‘odd-skipped related 1 (Drosophila)’ |
| ‘NM_003058’ | ‘SLC22A2’ | ‘solute carrier family 22 (organic cation transporter), |
| | | member 2’ |
| ‘NM_003058’ | ‘SLC22A2’ | ‘solute carrier family 22 (organic cation transporter), |
| | | member 2’ |
| ‘NM_153191’ | ‘SLC22A2’ | ‘solute carrier family 22 (organic cation transporter), |
| | | member 2’ |
| ‘NM_004378’ | ‘CRABP1’ | ‘cellular retinoic acid binding |
| | | protein 1’ |
| ‘NM_020208’ | ‘SLC6A20’ | ‘solute carrier family 6 (proline IMINO transporter), |
| | | member 20’ |
| ‘NM_012450’ | ‘SLC13A4’ | ‘solute carrier family 13 (sodium/sulfate symporters), |
| | | member 4’ |
| ‘NM_012450’ | ‘SLC13A4’ | ‘solute carrier family 13 (sodium/sulfate symporters), |
| | | member 4’ |
| ‘NM_033014’ | ‘OGN’ | ‘osteoglycin’ |
| ‘NM_014057’ | ‘OGN’ | ‘osteoglycin’ |
| ‘NM_000185’ | ‘SERPIND1’ | ‘serpin peptidase inhibitor, clade D (heparin cofactor), |
| | | member 1’ |
| ‘NM_000185’ | ‘SERPIND1’ | ‘serpin peptidase inhibitor, clade D (heparin cofactor), |
| | | member 1’ |
| ‘NM_00159’ | ‘AOX1’ | ‘aldehyde oxidase 1’ |
| ‘Contig30092_RC’ | ‘PRDM6’ | ‘PR domain containing 6’ |
| ‘NM_017565’ | ‘FAM20A’ | ‘family with sequence similarity 20, member A’ |
| ‘NM_024101’ | ‘MLPH’ | ‘melanophilin’ |
| ‘Contig56735_RC’ | ‘SPTLC3’ | ‘serine palmitoyltransferase, long chain base subunit 3’ |
| ‘NM_053277’ | ‘CLIC6’ | ‘chloride intracellular channel 6’ |
| ‘Contig44729_RC’ | ‘CLIC6’ | ‘WDNM1-like pseudogene’ |
| ‘NM_004415’ | ‘DSP’ | ‘desmoplakin’ |
| ‘NM_005982’ | ‘SIX1’ | ‘SIX homeobox 1’ |
| ‘NM_002593’ | ‘PCOLCE’ | ‘procollagen C-endopeptidase enhancer’ |
| ‘NM_015516’ | ‘TSKU’ | ‘tsukushi small leucine rich proteoglycan homolog |
| | | (Xenopus laevis)’ |
| ‘NM_002242’ | ‘KCNJ13’ | ‘potassium inwardly-rectifying channel, subfamily J, |
| | | member 13’ |
| ‘NM_005014’ | ‘OMD’ | ‘osteomodulin’ |
| ‘NM_016615’ | ‘SLC6A13’ | ‘solute carrier family 6 (neurotransmitter transporter, |
| | | GABA), member 13’ |
| ‘NM_016615’ | ‘SLC6A13’ | ‘solute carrier family 6 (neurotransmitter transporter, |
| | | GABA), member 13’ |
| ‘NM_203422’ | ‘LRRN4CL’ | ‘LRRN4 C-terminal like’ |
| ‘NM_004004’ | ‘GJB2’ | ‘gap junction protein, beta 2, 26 kDa’ |
| ‘NM_000612’ | ‘IGF2’ | ‘insulin-like growth factor 2 (somatomedin A)’ |
| ‘NM_002207’ | ‘ITGA9’ | ‘integrin, alpha 9’ |
| ‘NM_144716’ | ‘CCDC12’ | ‘coiled-coil domain containing 12’ |
| ‘NM_000954’ | ‘PTGDS’ | ‘prostaglandin D2 synthase 21 kDa (brain)’ |
| ‘NM_139005’ | ‘HFE’ | ‘hemochromatosis’ |
| ‘NM_139002’ | ‘HFE’ | ‘hemochromatosis’ |
| ‘NM_017614’ | ‘BHMT2’ | ‘betaine-homocysteine methyltransferase 2’ |
| ‘NM_032035’ | ‘LTBP2’ | ‘latent transforming growth factor beta binding protein 2’ |
| ‘Contig44040_RC’ | ‘IRX3’ | ‘iroquois homeobox 3’ |
| ‘NM_000104’ | ‘CYP1B1’ | ‘cytochrome P450, family 1, subfamily B, polypeptide 1’ |
| ‘NM_000104’ | ‘CYP1B1’ | ‘cytochrome P450, family 1, subfamily B, polypeptide 1’ |
| ‘NM_006770’ | ‘MARCO’ | ‘macrophage receptor with collagenous structure’ |
| ‘NM_006840’ | ‘LILRB5’ | ‘leukocyte immunoglobulin-like receptor, subfamily B (with |
| | | TM and ITIM domains), member 5’ |
| ‘X17653’ | ‘FCGR2B’ | ‘Fc fragment of IgG, low affinity IIb, receptor (CD32)’ |
| ‘NM_004001’ | ‘FCGR2B’ | ‘Fc fragment of IgG, low affinity IIb, receptor (CD32)’ |
| ‘NM_006691’ | ‘LYVE1’ | ‘lymphatic vessel endothelial hyaluronan receptor 1’ |
| ‘NM_016164’ | ‘LYVE1’ | ‘lymphatic vessel endothelial hyaluronan receptor 1’ |
| ‘NM_002438’ | ‘MRC1’ | ‘mannose receptor, C type 1’ |
| ‘Contig2930_RC’ | ‘DAB2’ | ‘disabled homolog 2, mitogen-responsive phosphoprotein |
| | | (Drosophila)’ |
| ‘NM_001343’ | ‘DAB2’ | ‘disabled homolog 2, mitogen-responsive phosphoprotein |
| | | (Drosophila)’ |
| ‘NM_001466’ | ‘FZD2’ | ‘frizzled homolog 2 (Drosophila)’ |
| ‘BC040697’ | ‘TBX18’ | ‘T-box 18’ |
| ‘NM_003373’ | ‘VCL’ | ‘vinculin’ |
| ‘Contig57359_RC’ | ‘VGLL3’ | ‘vestigial like 3 (Drosophila)’ |
| ‘NM_181526’ | ‘MYL9’ | ‘myosin, light chain 9, regulatory’ |
| ‘NM_002474’ | ‘MYH11’ | ‘myosin, heavy chain 11, smooth muscle’ |
| ‘NM_003186’ | ‘TAGLN’ | ‘transgelin’ |
| ‘NM_003289’ | ‘TPM2’ | ‘tropomyosin 2 (beta)’ |
| ‘NM_052966’ | ‘FAM129A’ | ‘family with sequence similarity 129, member A’ |
| ‘NM_000900’ | ‘MGP’ | ‘matrix Gla protein’ |
| ‘HSS00178724’ | ‘MGP’ | ‘UPF0632 protein A’ |
| ‘Contig45441_RC’ | ‘MGP’ | ‘hypothetical protein LOC284542’ |
| ‘NM_182487’ | ‘OLFML2A’ | ‘olfactomedin-like 2A’ |
| ‘NM_000089’ | ‘COL1A2’ | ‘collagen, type I, alpha 2’ |
| ‘Contig48518_RC’ | ‘SCUBE3’ | ‘signal peptide, CUB domain, EGF- |
| | | like 3’ |
| ‘NM_002404’ | ‘MFAP4’ | ‘microfibrillar-associated protein 4’ |
| ‘NM_000090’ | ‘COL3A1’ | ‘collagen, type III, alpha 1’ |
| ‘AL137566’ | ‘PGR’ | ‘progesterone receptor’ |
| ‘NM_004417’ | ‘DUSP1’ | ‘dual specificity phosphatase 1’ |
| ‘NM_015429’ | ‘ABI3BP’ | ‘ABI family, member 3 (NESH) binding protein’ |
| ‘NM_001847’ | ‘COL4A6’ | ‘collagen, type IV, alpha 6’ |
| ‘Contig45367_RC’ | ‘BNC2’ | ‘basonuclin 2’ |
| ‘NM_017637’ | ‘BNC2’ | ‘basonuclin 2’ |
| ‘Contig43613_RC’ | ‘BNC2’ | ‘basonuclin 2’ |
| ‘Contig47865’ | ‘GPX8’ | ‘glutathione peroxidase 8 (putative)’ |
| ‘NM_001393’ | ‘ECM2’ | ‘extracellular matrix protein 2, female organ and adipocyte |
| | | specific’ |
| ‘NM_020311’ | ‘CXCR7’ | ‘chemokine (C-X-C motif) receptor 7’ |
| ‘NM_152459’ | ‘C16orf89’ | ‘chromosome 16 open reading frame 89’ |
| ‘NM_032348’ | ‘MXRA8’ | ‘matrix-remodelling associated 8’ |
| ‘NM_002889_sat’ | ‘RARRES2’ | ‘retinoic acid receptor responder (tazarotene induced) 2’ |
| ‘NM_002889’ | ‘RARRES2’ | ‘retinoic acid receptor responder (tazarotene induced) 2’ |
| ‘NM_152403’ | ‘EGFLAM’ | ‘EGF-like, fibronectin type III and laminin G domains’ |
| ‘NM_001608’ | ‘ACADL’ | ‘acyl-Coenzyme A dehydrogenase, long chain’ |
| ‘NM_002508’ | ‘NID1’ | ‘nidogen 1’ |
| ‘Contig37571_RC’ | ‘THSD4’ | ‘thrombospondin, type I, domain containing 4’ |
| ‘Contig56678_RC’ | ‘THSD4’ | ‘thrombospondin, type I, domain containing 4’ |
| ‘Contig43710_RC’ | ‘THSD4’ | ‘hypothetical LOC100130938’ |
| ‘AF086149’ | ‘THSD4’ | ‘similar to meteorin, glial cell differentiation regulator-like’ |
| ‘Contig55228_RC’ | ‘FAM46C’ | ‘family with sequence similarity 46, member C’ |
| ‘AJ420583’ | ‘FAM46A’ | ‘family with sequence similarity 46, member A’ |
| ‘NM_017633’ | ‘FAM46A’ | ‘family with sequence similarity 46, member A’ |
| ‘NM_153206’ | ‘AMICA1’ | ‘adhesion molecule, interacts with CXADR antigen 1’ |
| ‘NM_022121’ | ‘PERP’ | ‘PERP, TP53 apoptosis effector’ |
| ‘NM_021005’ | ‘NR2F2’ | ‘nuclear receptor subfamily 2, group F, member 2’ |
| ‘NM_021005’ | ‘NR2F2’ | ‘nuclear receptor subfamily 2, group F, member 2’ |
| ‘NM_006486’ | ‘FBLN1’ | ‘fibulin 1’ |
| ‘NM_001996’ | ‘FBLN1’ | ‘fibulin 1’ |
| ‘NM_006487’ | ‘FBLN1’ | ‘fibulin 1’ |
| ‘NM_145015’ | ‘MRGPRF’ | ‘MAS-related GPR, member F’ |
| ‘NM_032876’ | ‘JUB’ | ‘jub, ajuba homolog (Xenopus laevis)’ |
| ‘NM_058172’ | ‘ANTXR2’ | ‘anthrax toxin receptor 2’ |
| ‘NM_007129’ | ‘ZIC2’ | ‘Zic family member 2 (odd-paired homolog, Drosophila)’ |
| ‘NM_000362’ | ‘TIMP3’ | ‘TIMP metallopeptidase inhibitor 3’ |
| ‘NM_153703’ | ‘PODN’ | ‘podocan’ |
| ‘NM_006522’ | ‘WNT6’ | ‘wingless-type MMTV integration site family, member 6’ |
| ‘NM_004472’ | ‘FOXD1’ | ‘forkhead box D1’ |
| ‘NM_015493’ | ‘KANK2’ | ‘KN motif and ankyrin repeat domains 2’ |
| ‘NM_002725’ | ‘PRELP’ | ‘proline/arginine-rich end leucine-rich repeat protein’ |
| ‘AK021858’ | ‘FOXC1’ | ‘forkhead box C1’ |
| ‘NM_001453’ | ‘FOXC1’ | ‘forkhead box C1’ |
| ‘Contig36522_RC’ | ‘TBX15’ | ‘T-box 15’ |
| ‘NM_001920’ | ‘DCN’ | ‘decorin’ |
| ‘NM_133503’ | ‘DCN’ | ‘decorin’ |
| ‘NM_001718’ | ‘BMP6’ | ‘bone morphogenetic protein 6’ |
| ‘NM_004107’ | ‘FCGRT’ | ‘Fc fragment of IgG, receptor, transporter, alpha’ |
| ‘NM_002101’ | ‘GYPC’ | ‘glycophorin C (Gerbich blood group)’ |
| ‘NM_016815’ | ‘GYPC’ | ‘glycophorin C (Gerbich blood group)’ |
| ‘NM_006682’ | ‘FGL2’ | ‘fibrinogen-like 2’ |
| ‘NM_005202’ | ‘COL8A2’ | ‘collagen, type VIII, alpha 2’ |
| ‘NM_001562’ | ‘IL18’ | ‘interleukin 18 (interferon-gamma-inducing factor)’ |
| ‘NM_030582’ | ‘COL18A1’ | ‘collagen, type XVIII, alpha 1’ |
| ‘NM_000428’ | ‘LTBP2’ | ‘latent transforming growth factor beta binding protein 2’ |
| ‘X02761_sat’ | ‘FN1’ | ‘fibronectin 1’ |
| ‘NM_002026’ | ‘FN1’ | ‘fibronectin 1’ |
| ‘NM_001849’ | ‘COL6A2’ | ‘collagen, type VI, alpha 2’ |
| ‘NM_021738’ | ‘SVIL’ | ‘supervillin’ |
| ‘NM_003174’ | ‘SVIL’ | ‘supervillin’ |
| ‘NM_004696’ | ‘SLC16A4’ | ‘solute carrier family 16, member 4 (monocarboxylic acid |
| | | transporter 5)’ |
| ‘NM_004696’ | ‘SLC16A4’ | ‘solute carrier family 16, member 4 (monocarboxylic acid |
| | | transporter 5)’ |
| ‘NM_003412’ | ‘ZIC1’ | ‘Zic family member 1 (odd-paired homolog, Drosophila)’ |
| ‘NM_006492’ | ‘ALX3’ | ‘ALX homeobox 3’ |
| ‘NM_000587’ | ‘C7’ | ‘complement component 7’ |
| ‘NM_014350’ | ‘TNFAIP8’ | ‘tumor necrosis factor, alpha-induced protein 8’ |
| ‘NM_001497’ | ‘B4GALT1’ | ‘UDP-Gal: betaGlcNAc beta 1,4-galactosyltransferase, |
| | | polypeptide 1’ |
| ‘NM_000062’ | ‘SERPING1’ | ‘serpin peptidase inhibitor, clade G (C1 inhibitor), |
| | | member 1’ |
| ‘M62896’ | ‘ANXA2P1’ | ‘annexin A2 pseudogene 1’ |
| ‘hCT2336680’ | ‘ANXA2P2’ | ‘annexin A2 pseudogene 2’ |
| ‘NM_004039’ | ‘ANXA2’ | ‘annexin A2’ |
| ‘NM_013451’ | ‘MYOF’ | ‘myoferlin’ |
| ‘X56210_sat’ | ‘CFHR1’ | ‘complement factor H-related 1’ |
| ‘NM_000186_sat’ | ‘CFH’ | ‘complement factor H’ |
| ‘M65292_sat’ | ‘CFHR1’ | ‘complement factor H-related 1’ |
| ‘NM_000186’ | ‘CFH’ | ‘complement factor H’ |
| ‘NM_002113’ | ‘CFHR1’ | ‘complement factor H-related 1’ |
| ‘X56210’ | ‘CFHR1’ | ‘complement factor H-related 1’ |
| ‘NM_002546’ | ‘TNFRSF11B’ | ‘tumor necrosis factor receptor superfamily, member 11b’ |
| ‘NM_002546’ | ‘TNFRSF11B’ | ‘tumor necrosis factor receptor superfamily, member 11b’ |
| ‘NM_000627’ | ‘LTBP1’ | ‘latent transforming growth factor beta binding protein 1’ |
| ‘NM_003380’ | ‘VIM’ | ‘vimentin’ |

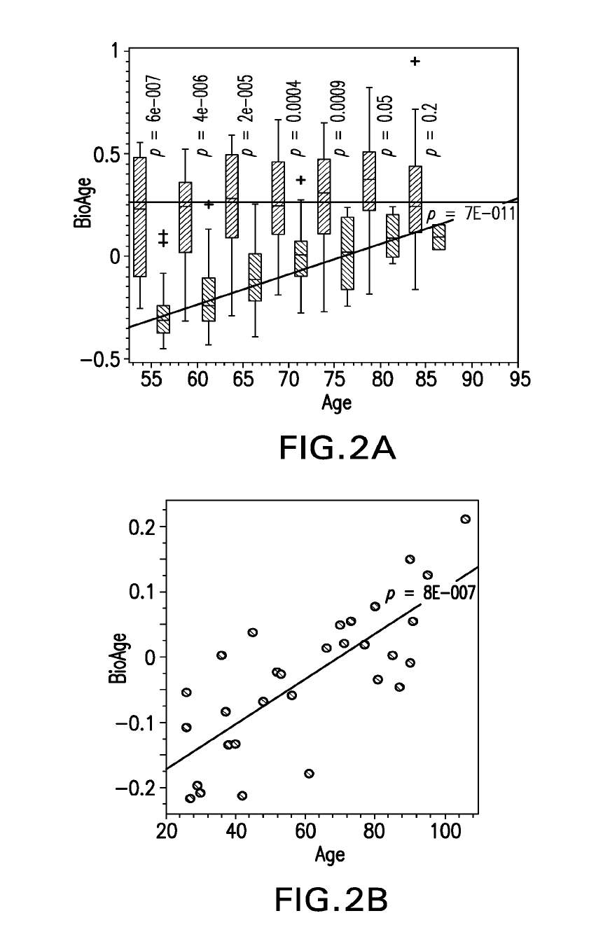

It is useful to ascribe a signature score based on the average expression levels for all included genes as a composite measure of the signature. Applicants refer to the PC1 signature score herein as BioAge (biological age). Without wishing to be bound by any theory, Applicants believe that the BioAge signature score (herein the “Score”) of each brain tissue sample is a more precise and objective measure of its aging level than chronological age. Most of the AD subjects attained much larger values for BioAge than normal subjects (AUROC=0.92). Comparison of the Score for BioAge for AD and non-demented individuals at different chronological age groups revealed a very significant difference at younger ages, which decreased in chronologically older age groups. While the Score for BioAge of non-demented individuals gradually increased with age, AD patients showed consistently higher Scores for BioAge regardless of chronological age (FIG. 2A). The extrapolated Scores for normal subjects would reach the average AD Score at a chronological age of 100 years. The most advanced AD brains had Scores for BioAge corresponding to an extrapolated chronological age of 140 years in normal subjects.

As an independent test of the power of BioAge, that is, the average gene expression or Score for this biomarker, to predict normal chronological age, Applicants applied this biomarker to a cohort of prefrontal cortex samples from non-demented individuals (Gene Expression Omnibus dataset, GSE1572) that were used to qualitatively describe aging in an earlier study (Lu, T., et al., 2004, Nature, 429: 883-891). The BioAge Score in these samples strongly and significantly correlated with the chronological age of the subjects (ρ=0.75, p=8E-7, FIG. 2B). In addition, BioAge corresponded to the second principal component in the GSE 1572 dataset (ρ=0.90, p=4E-11), validating that aging was a major reproducible source of variance in gene expression in PFC. Prediction of chronological age using gene expression was recently proposed in the literature, Cao, K., et al., 2010, PLoS One, 5: e13098).

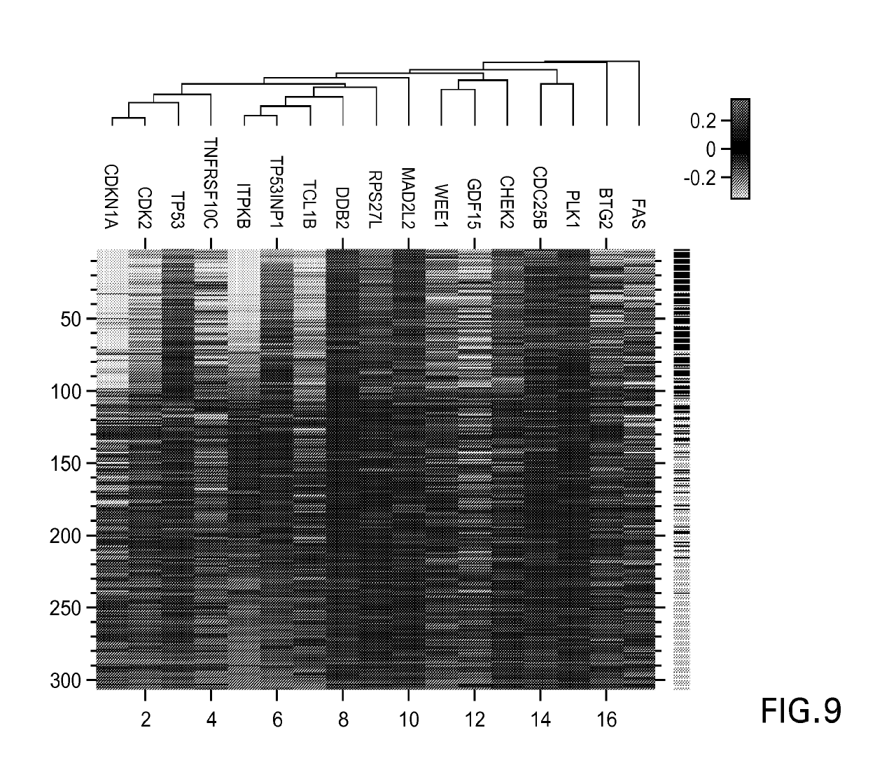

The massive gene expression changes associated with aging that Applicants detected involved a constellation of biological processes. Gene set annotation analysis revealed that the genes down-regulated with increasing BioAge showed significant enrichment for neuronal and synaptic processes, possibly reflecting neuronal depletion or loss of plasticity (data not shown). The up-regulated processes include lipid metabolism, FAK signaling and axon guidance, as well as the glial marker, GFAP (Table 2). In agreement with an earlier analysis of aging signatures observed in normal brains (Yanker, B. A., et al., 2004, Nature, 429:883-891; Lu, T., et al., 2004), the up-regulated genes contain several oncogenes (for example, TP53, PI3K, PTEN), shown to be strongly correlated with BioAge in FIG. 9.

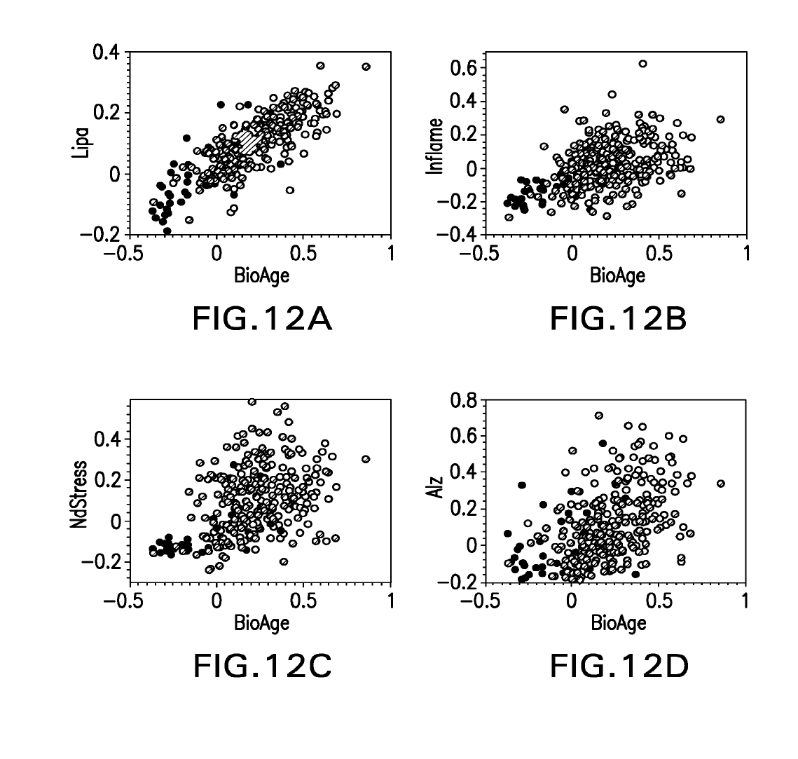

Applicants also found that the up-regulated portion of the BioAge biomarker could be further dissected using a metagene discovery approach where genes significantly associated with a disease trait and a very strong Pearson correlation with each other are treated as a single unit (Tamayo, P. et al., 2007, Proc. Natl. Acad. Sci. U.S.A., 104:5959-5964; Carvalho, C., et al., 2008, J. Am. Statistical Assoc., 103:1438-1456; Oldham, M. C. et al., 2008, Nat. Neurosci., 11: 1271-1282; Miller, J. A. et al., 2008, J. Neurosci., 28: 1410-1420. Applicants selected samples with relatively low BioAge (BioAge <0) and found a large metagene with exceptionally high mutual correlation between the genes. The range of expression values for the genes comprising the metagene in these samples corresponded to an average three fold up-regulation early in the aging process. This metagene was much more coherent in normal samples than in AD samples. Applicants named this metagene “Lipa” (Table1) because it included APOE, PPARA, γ-protocadherins, and other genes involved in lipid metabolism, amino acid metabolism and cell adhesion. Other notable Lipa genes included HES1, TGFB2, NTRK2, and WIF 1. FIGS. 12A-12D illustrate the relationship between metagene-based biomarkers and selected component genes mentioned herein.

### Disease-Specific Biomarkers

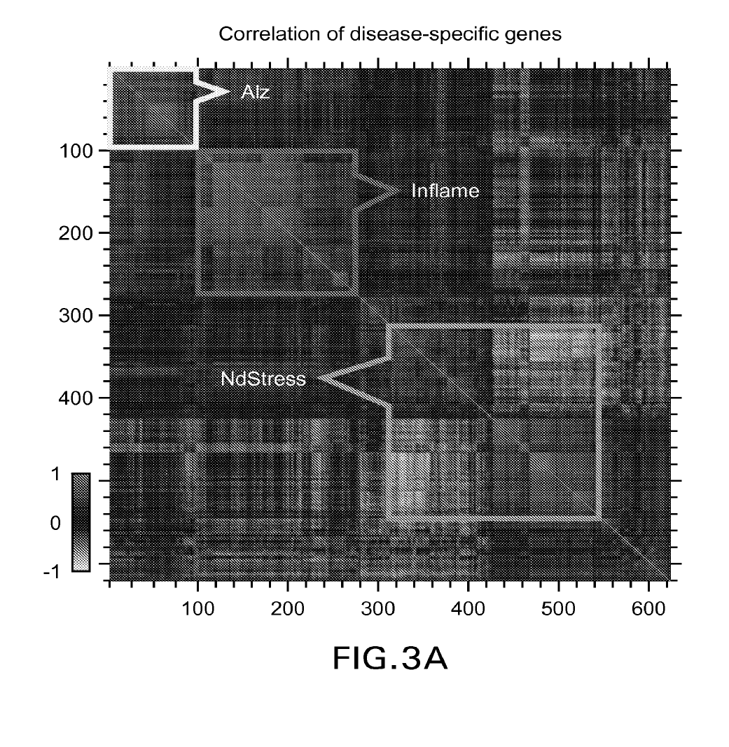

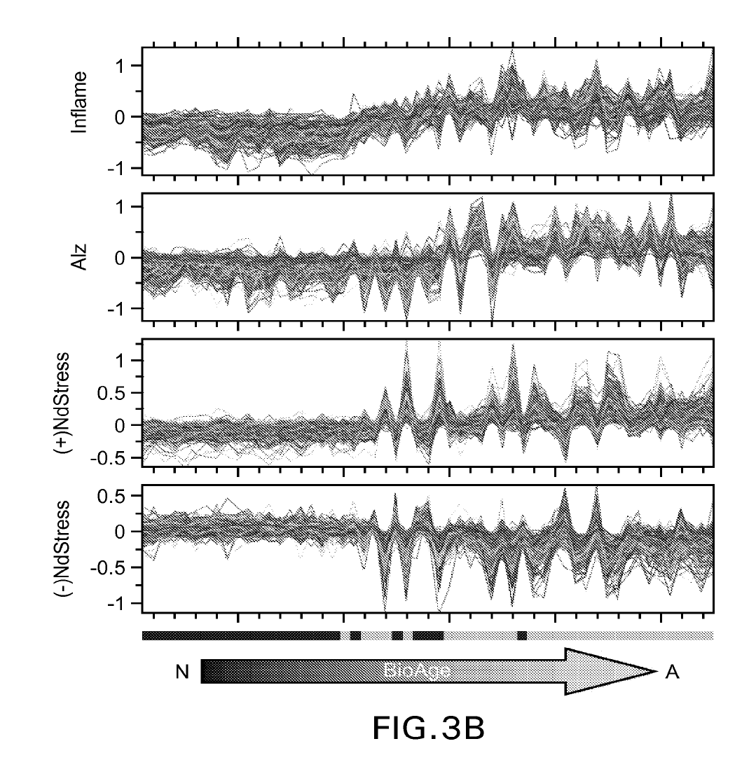

The higher BioAge score of AD patients explained more than 50% of the differential expression between normal (non-demented) and AD cohorts. In the range of BioAge scores in which AD and normal individuals overlap, there was a significant residual differential expression, composed of several distinct sub-patterns that explain a large fraction of the normal-to-AD variance. Applicants focused on 88 AD and 43 normal brain samples with matched moderate levels of BioAge between −0.1 and 0.3. Applicants identified 4,500 genes that are differentially expressed between the two cohorts (ANOVA p<0.005, absolute fold change >10%, FDR <0.1). FIGS. 3A and 3B show the supervised metagene analysis of these genes based on clustering using gene-gene correlation as a distance measure (see Example ?). In this analysis, the three most regulated metagenes responsible for the majority of the gene expression differences associated with the disease were identified.

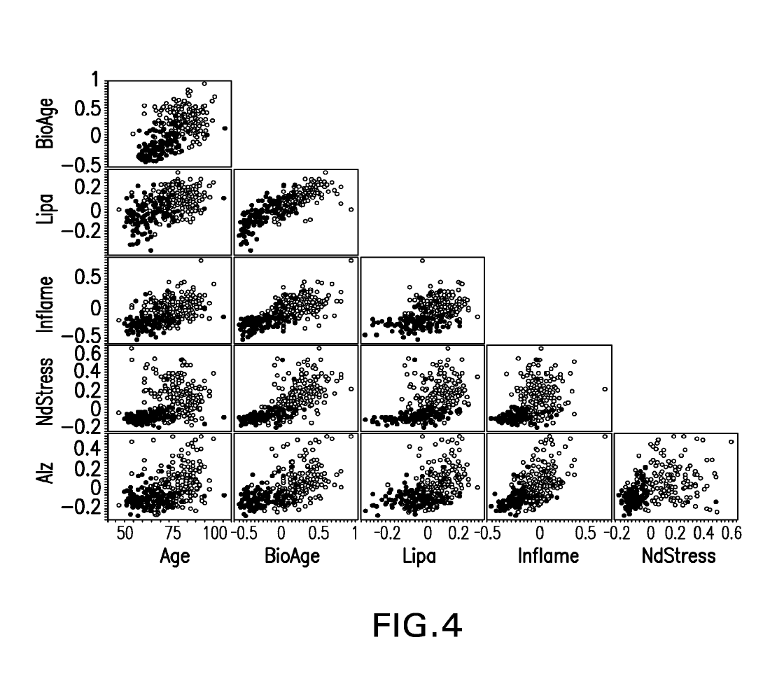

The first and the largest group of about 2,000 genes, herein defined as “NdStress,” was associated with various metabolic disruptions. This signature contained some genes that were up-regulated (+NdStress, Table 5) and others that were down-regulated (−NdStress, Table 6) in AD subjects. The expression of these genes was maintained in a relatively stable narrow range in normal brains with low BioAge with relatively low coherence (FIG. 3A), while in AD subjects, the expression of these genes varied dramatically and was highly correlated (FIGS. 3A and 3B, Table 8). Although the plethora of biological pathways reflected in this large biomarker precluded significant enrichment of an individual pathway after correcting for multiple testing, the up-regulated (+NdStress, Table 5) arm of this signature contained multiple heatshock and proteosome proteins, such as HSP1A1, STIP1, HSP1B1, PSMB1/D6, and the TGFβ signaling proteins SMAD2 and SMAD4 (FIGS. 12A-12D). The down-regulated (−NdStress, Table 6) arm of NdStress is enriched in genes involved in folate metabolism, such as DHFRL1, MTR and FPGS, possibly related to the alterations in folate and homocysteine observed in AD patients. FIG. 4 shows the relationship between NdStress and BioAge, which moderately correlated in AD samples (ρ=0.53, p<1E-13). At the same time, NdStress and chronological age correlated negatively (ρ=−0.14, p=0.05). This metagene score explained 22% of variance in differentially expressed genes and demonstrated AUROC of 0.75 in separating AD and normal samples.

TABLE 8

| Signature score | BioAge | Inflame | NdStress | Alz |
|---|---|---|---|---|
| Differential variance explained, | 0.42 | 0.23 | 0.29 | 0.17 |
| all samples | | | | |
| Differential variance explained, | 0.09 | 0.11 | 0.22 | 0.06 |
| BioAge-matched samples | | | | |
| AUROC, all samples | 0.92 | 0.88 | 0.89 | 0.81 |
| AUROC, BioAge-matched | 0.69 | 0.72 | 0.75 | 0.69 |
| samples | | | | |
| Coherence in normal samples | 0.80 | 0.80 | 0.57 | 0.64 |
| Coherence in AD samples | 0.84 | 0.82 | 0.76 | 0.81 |

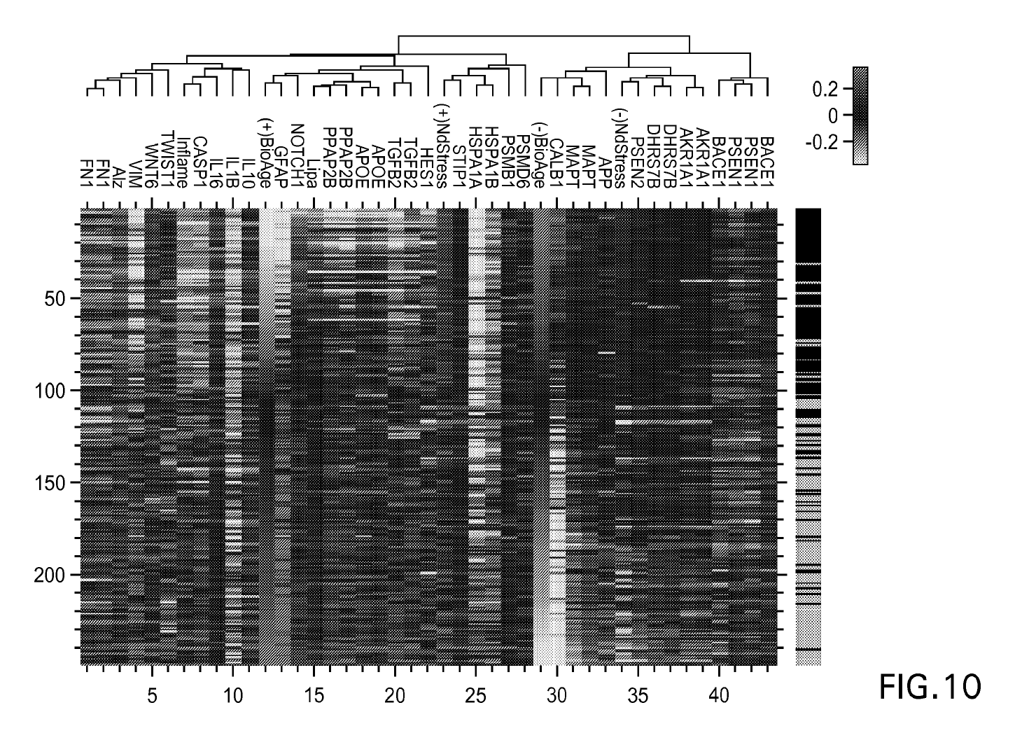

The second metagene, herein defined as “Alz,” consisted of about 200 genes up-regulated in AD (FIGS. 3A and 3B, Table 7). This signature was enriched in genes involved in cell communication/adhesion, fibrosis, mesoderm development and ossification such as numerous collagen genes, BMP genes, CTSK, MFAP2/4, FN1, VIM, WNT6 and TWIST1 (FIG. 10, Table 9). This signature also contained several prostaglandin synthases and receptors. Alz positively correlated with both BioAge (ρ=0.40, p<1E-7) and chronological age (ρ=0.23, p=0.002), see also FIG. 4. This metagene score explained 6% of variance in differentially expressed genes and demonstrated AUROC of 0.69 in separating AD and normal samples.

TABLE 9

| Biomarker | Selected enriched pathways |
|---|---|
| Lipa | Cell adhesion**; RXR function**; fatty acid metabolism**; amino acid |
| | metabolism** |
| (+) BioAge | Molecular mechanisms of cancer*; lipid metabolism*; FAK signaling*; axon |
| | guidance* |
| (−) BioAge | Neuronal activities**, synaptic transmission**; axonal guidance*; long term |
| | potentiation/depression**; molecular mechanisms of cancer*; Ca/Glutamate/ |
| | MAPK signaling* |
| Inflame | Innate immune response**, apoptosis**, macrophage** |
| (+) NdStress | Stress response^{#}; PPAR RXR acivation^{#}, glucocorticoid signaling^{#} |
| (−) NdStress | Metabolic pathways**; folate metabolism^{#} |
| Alz | Cell communication**; fibrosis**; mesoderm development**; cell adhesion**; |
| | ossification* |

**Bonferroni corrected Hypergeometric p-value < 0.05
*Bonferroni corrected Hypergeometric p-value < 0.1
^{#}Bonferroni corrected Hypergeometric p-value < 0.5

Finally, a small, but exceptionally tightly correlated, metagene herein defined as “Inflame” (Table 4) contained about 250 genes upregulated with AD including many inflammation markers, such as IL1B, 1L10, IL16, IL18, and HLA genes, as well as markers of macrophages, such as VSIG4, SLC11A1, and apoptosis, such as CASP1/4, TNFRSF1B (p75 death receptor) (FIGS. 3A and 3B, Table 9). The Inflame score explained 11% of variance in differentially expressed genes and positively correlated with BioAge (ρ=0.47, p=1E-10) and chronological age (ρ=0.28, p<0.001) in AD subjects. When used as a classifier, the Inflame score was capable of discriminating AD and normal brain with AUROC of 0.69. These genes maintained their mutual correlation in both normal and AD subjects, but reached significantly higher levels in AD.

FIG. 4 shows the interplay between the biomarkers discussed above and complex causal relationships between them. For example, the elevation of Inflame preceded the elevation of NdStress, because there are no samples with high NdStress, but low Inflame. However, the correlation between NdStress and Inflame is low in AD samples where NdStress is active (ρ=0.21, p=0.004). Applicants also observed a very low correlation between NdStress and Alz (ρ=0.21, p=0.004) and moderate correlation between Alz and Inflame (ρ=0.47, p=1E-11) in AD samples.

### Systemic and Localized Brain Changes

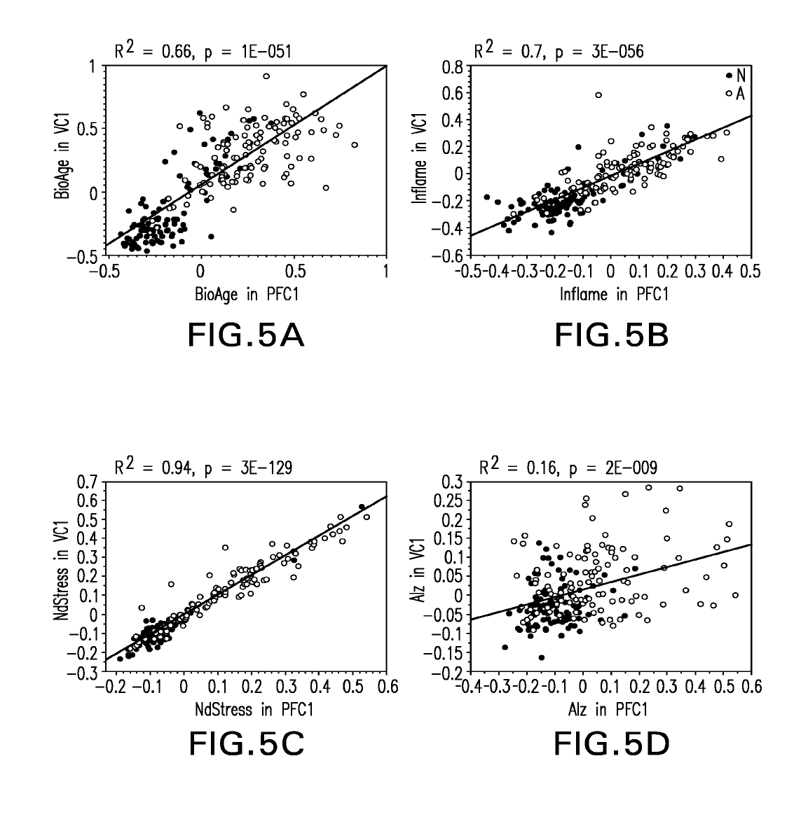

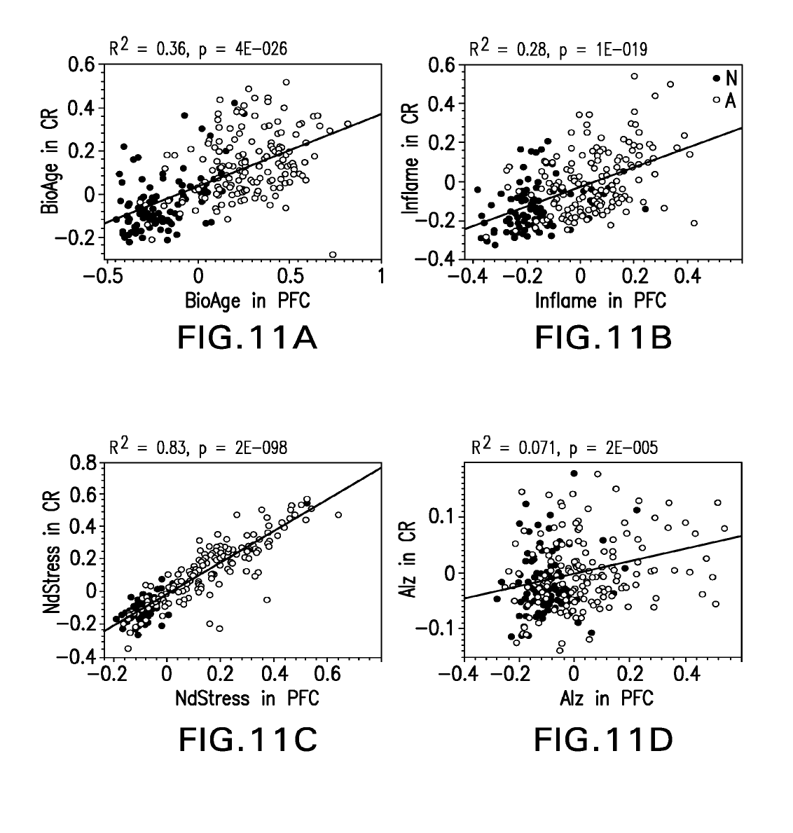

A unique feature of this dataset is the availability of samples from different brain regions belonging to the same individual. All biomarkers determined from prefrontal cortex (PFC) samples were tested for coherence in visual cortex (VC) and cerebellum (CR) samples. Applicants confirmed that BioAge and the disease-specific signatures were still expressed coherently and differentially between normal and AD subjects. Applicants then performed direct correlation analysis between the signature scores in different regions (FIGS. 5A-5D and 11A-11D). The biomarker, BioAge, demonstrated a relatively high correlation of 0.81 between VC1 and PFC1, with residual differences possibly reflecting different levels of aging between the brain regions. The Lipa biomarker also demonstrated a high correlation of 0.80 between these regions. Applicants determined that the correlation between Inflame scores in PFC 1 and VC1 was equal to 0.83. The highest correlation of 0.93 between PFC1 and VC1 was observed in the NdStress biomarker. Similar results were obtained between PFC 1 and CR1 (FIGS. 11A-11D). Without wishing to be bound by any theory, Applicants believe this exceptionally high level of correlation between the regions is likely explained by the systemic nature of inflammation and metabolic regulation that span diverse brain regions. Conversely, Alz scores did not show any significant correlations between regions in AD subjects, suggesting that this biomarker was confined to affected brain regions (Braak, H. and Braak, E., 1991, Acta Neuropathol., 82: 239-259) and more specifically related to AD pathogenesis (FIGS. 5A-5D and 11A-11D).

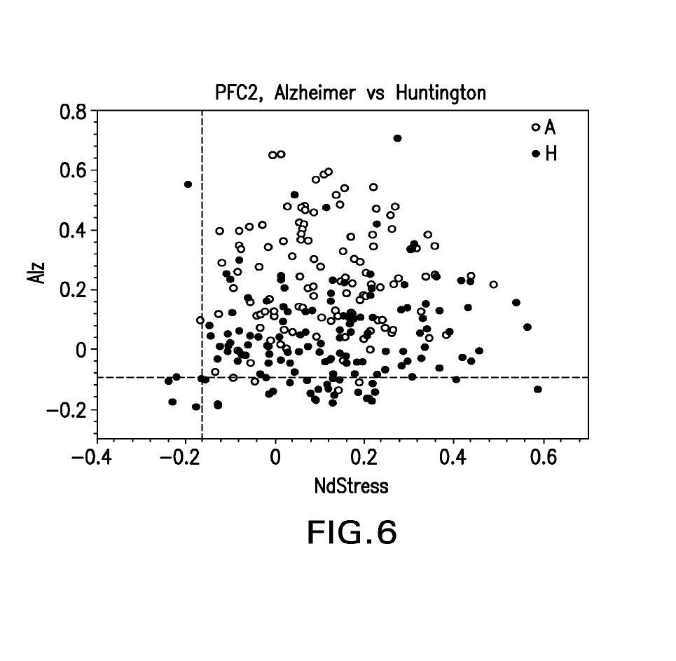

Furthermore, the disease biomarkers were fully validated in a hold-out set of samples (Phase 2), which in addition contained some Huntington disease (HD) subjects. As shown in FIGS. 12A-12D, BioAge, NdStress, and Inflame were significantly elevated in both AD and HD samples (p<0.01). In general, these biomarkers reached similar average levels in AD and HD samples in all profiled brain regions. However, in PFC2 the average BioAge reached in HD subjects was significantly lower than that of AD subjects (p=1E-17). These biomarkers, therefore, appear to capture general systemic neurodegenerative processes rather than being specific to AD. The most striking difference between AD and HD subjects was reflected in the Alz biomarker, which again was specific to the presence of AD and was not significantly elevated in any brain region in HD samples (FIG. 6).

Comparison with Brain Transcriptome

Consistent patterns of gene expression were recently observed by coexpression analyses in several large cohorts of brain samples from non-demented individuals (Oldham, et al., 2008, Nat. Neurosci., 11: 1271-1282). Applicants discovered several, reproducible metagenes, defined herein as “brain transcriptome modules,” some of which have been associated with genes expressed in specific brain cell types. In particular, the most reproducible modules, M4/5, M9, M15, and M16 (data not shown), were associated with microglia, oligodendrocytes, astrocytes, and neurons, respectively, in the cited work (Oldham, et al., 2008, Nat. Neurosci., 11: 1271-1282). Applicants validated the coherence of these modules in the Harvard Brain Tissue Resource Center (HBTRC) (McLean Hospital, Belmont, Mass.) dataset by metagene analysis and found that more than 90% of the genes comprising these modules strongly correlated with each other (ρ>0.7) within normal subjects. This analysis supports the finding that the latent structure of gene expression in cortex was preserved in dataset used herein.

In addition, we compared the gene expression profiling captured by the brain transcriptome modules with the biomarker, BioAge, and the disease-specific patterns discovered herein. Applicants found a strong correlation between M4/5 associated with microglia and the Inflame biomarker (ρ=0.92). In addition, “astrocytic” M15 correlates with BioAge (ρ=0.83) and “neuronal” M16 negatively correlates with BioAge (ρ=−0.93). Applicants also found that none of the major brain transcriptome modules strongly correlated with either the neurodegenerative NdStress or the AD specific Alz biomarkers. This confirms that these expression patterns are novel patterns that can only be detected in brains of those individuals affected by the disease.

### Systemic and Localized Molecular Changes in AD

This genome-wide gene expression profiling study of a large cohort of AD and normal aging brains revealed large groups of genes that vary as a function of age and disease status. When the hundreds of gene expression values contained in each of these sets are converted into a single quantitative trait, new molecular biomarkers of biological aging and disease progression emerge. The transcriptional profiles of AD brains were profoundly different from those in non-demented individuals, with thousands of genes differing in their levels of expression between the two cohorts. To reduce the complexity of the observed changes, Applicants focused on key gene expression patterns that explained the most variability across the cohorts. Applicants have found that the most significant pattern in terms of variance explained, both within and between the AD and non-demented cohorts, was BioAge, a biomarker of the level of biological aging in the brain. BioAge captured the extent of gradual molecular changes in the normal aging brain by averaging the gene expression changes associated with a multitude of synchronous physiological events. BioAge can be accurately and reliably assigned to each sample in the dataset and used to describe the molecular state of the brain in the same way as other clinical and physiological measurements are used by one of ordinary skill in the art.

Genes up-regulated with BioAge are associated with activation of cell cycle regulation pathways, lipid metabolism and axon guidance pathways (Table 2). Misexpression of cell cycle genes in post-mitotic neurons has been observed in aging and in AD subjects and has been suggested to be an important mechanism of neurodegeneration (Woods, et al., 2007, Biochim. Biophs. Acta, 1772: 503-508; Bonda, et al., 2010, Neuropathol. Appl. Neurobiol., 36: 157-163). The enrichment for oncogenes within this set is consistent with biological responses to genotoxic stress activated during aging in an increasingly larger population of brain cells. Genes down-regulated with BioAge were associated with a decrease in neuronal activity. Most of these genes maintained a strong correlation (connectivity) with BioAge throughout the entire range of the biomarker. This implies that the core of biological aging is one gradual change rather than several distinct transitions.

Contrary to most aging patterns, a significant loss of connectivity with aging was observed for the Lipa metagene (Table 1) that included APOE, HES1, and TGFB2 (FIG. 10). APOE and most of the other Lipa genes were expressed at high levels in all AD patients and some normal individuals. This suggests that up-regulation of lipid metabolism happens sometime early in the aging process and that activation of APOE and changes in lipid metabolism are early precursors of disease, possibly related to engagement of protection mechanisms.

Applicants have also found three other distinct disease-specific patterns. The biomarker, NdStress, which included both up- (+NdStress, Table 5) and down-regulated (−NdStress, Table 6) genes, dominated differential expression between AD and non-demented brains matched for BioAge score. The up-regulated genes contained multiple heatshock and proteasome proteins. Activation of these pathways may reflect the response to disease-related stress. Another set of genes in this module are cell cycle genes indicative of cell cycle arrest or apoptosis. The down-regulated (−NdStress, Table 6) arm of NdStress was enriched in one-carbon/folate metabolism genes and could underlay the perturbations in folic acid and one-carbon metabolism that are one of the earliest biomarkers associated with neurodegenerative disorders including AD (Kronenberg, et al., 2009, Curr. Mol. Med., 9: 315-23; Van Dam, F. and Van Gool, W. A., 2009, Arch. Gerontol. Geriatr., 48: 425-30; McCampbell, A. et al., 2011, J. Neurochem., 116, 82-92).

The second largest disease-specific pattern, Alz (Table 7), contained genes associated with cell adhesion, migration, morphogenesis. This biomarker prominently featured genes characteristic of epithelial-to-mesenchymal transition (EMT), such as VIM, TWIST1, and FN1 (Kalluri, R. and Weinberg, R. A., 2009, J. Clin. Invest., 119: 1420-8) (FIG. 10). The connection of Alz with EMT suggests a major transformation in brain tissue physiology including changes in receptor signaling, growth factor dependence, and cell adhesion during the disease. The third disease-specific biomarker, Inflame, which reflects chronic neuro-inflammation (Jakob-Roetne, R. and Jacobsen, H., 2009, Angew. Chem. Int. Ed. Engl., 48: 3030-3059; Eikelenboom, P. et al., 2006, J. Neural. Transm., 113: 1685-95), suggests a similarity between AD with other examples of EMT type 2, such as tissue fibrosis, where chronic inflammation and up-regulation of TGFB2 contribute to pathogenesis (Kalluri and Weinberg, 2009). The levels of Alz in AD are much higher than in unaffected brain regions or in the PFC of HD, suggesting that these gene expression changes are not generally reflecting neuro-degeneration, but rather relate to AD pathology.

Further, BioAge and Inflame are consistent with published analysis of healthy brain transcriptome and associated with neuronal, astrocytic, and microglial modules (Oldham, et al., 2008, Nat. Neurosci., 11:1271-1282). Importantly, Applicants found that NdStress and Inflame have virtually identical scores in different regions from the same individual. This suggests they measure systemic changes in brain tissue that happen across multiple cell types and layers and are independent of the diverse morphology and makeup of different brain regions. Alz scores, on the other hand, are not the same across all brain regions and had the highest levels in prefrontal cortex, indicating a local rather than systemic nature of EMT.

### Alzheimer Disease Progression Model

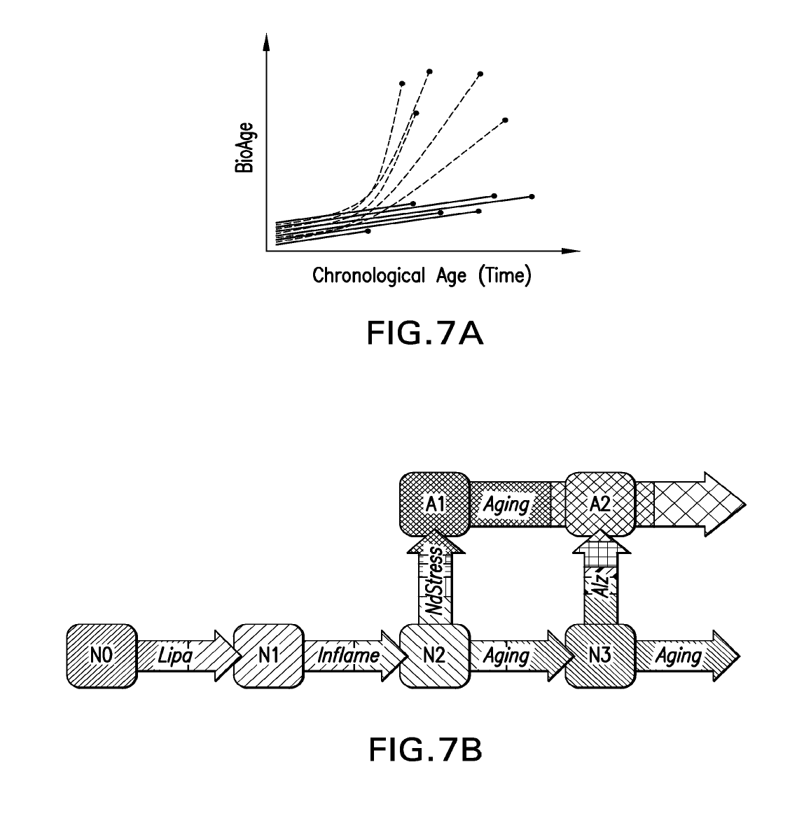

Applicants' analysis of gene expression changes in the brains of AD patients confirms that AD is both similar and distinct from the process of normal aging. Although each brain was captured only in a particular (postmortem) state and was not studied longitudinally, Applicants can assemble these data as a function of time to propose a few generalized aging trajectories (FIG. 7A). BioAge and chronological age showed a significant association in non-demented individuals and no association in AD patients, who had consistently high BioAge scores regardless of their chronological age. Applicants attributed this observation to a difference in the strength of the aging drivers, distribution of the aging rates, and different causes of death in the two cohorts. In non-demented individuals, the drivers of aging were weak. The rates of aging were relatively slow and consistent across the population and, in the absence of unnatural causes, death was likely related to aging issues other than the health of the brain. Since non-demented individuals likely died from causes largely unrelated to neurodegeneration, each individual death is conceptually a random event along the generalized brain aging trajectory. In AD patients, the drivers of aging were stronger and variable across the cohort and the death was generally related to the health of the brain, that became incompatible with life regardless of the chronological age. The extrapolated BioAge of normal patients would not reach the highest AD levels until the age of 140 years. Thus, AD can be viewed as an aberrant aging of the brain, which retains the gene expression hallmarks of normal aging combined with additional patterns associated with pathological drivers of the disease and response of the brain tissue to disease-related processes.

For AD patients, the studies herein are missing early stages of the aging trajectory and can only observe late stages with terminal high BioAge. Unlike the normal cohort that can be represented by a single trajectory, the AD cohort covers a family of trajectories with different rates of biological aging. Patients with a fast rate of biological aging would succumb to disease at younger ages and generally would have higher levels of BioAge relative to their chronological age in the early phases of disease. However, since the studies herein did not include longitudinal specimens from subjects before they developed the disease, a second biomarker was required to explain disease progression rates after BioAge is maximal. The expression profile of NdStress fits the properties expected of this progression rate biomarker as it was highest level in chronologically young AD patients and it significantly correlates with (+) BioAge and (−) chronological age. Alz, on the other hand, is the highest in chronologically older patients and does not correlate with BioAge. Thus, patients with high NdStress likely have more accelerated aging trajectories than patients with high Alz. The older chronological age of Alz onset may suggest that the acceleration of BioAge due to Alz does not occur until the level of BioAge of the brain reaches a certain threshold. The quantitative assessment of the brain biological age in terms of BioAge and the rate of its disease-related acceleration in terms of NdStress are two critical hypotheses proposed in this work.

Another way to look at the aging trajectory is to model it as a set of molecular transitions that lead to changes in BioAge. Examination of biomarker scores for BioAge-low brains in FIG. 4 suggests that up-regulation and disruption of the Lipa biomarker happens very early in the aging process because most of these samples have the lowest Lipa scores in the cohort. Comparing Inflame with Lipa and BioAge shows that activation of the inflammation biomarker also happens early in the aging process but not as early as Lipa activation because there are BioAge-young patients with high Lipa score but low Inflame. These and other observations can be summarized in the form of a state transition model shown in FIG. 7B. Aging starts with up-regulation of APOE and other lipid metabolic genes, together with Notch and TGFβ, signaling signifying the transition from N0 to N1. The subsequent up-regulation of the Inflame biomarker is associated with transition from N1 to N2. The brains in these states were diagnosed as normal because the subjects did not yet exhibit any cognitive impairment associated with AD. The next transition, from N2 to A1, is associated with massive disruptions in metabolic pathways and marked acceleration of aging follows. However, some brains avoid transitioning to A1 and continue to age into N3. Another transition to the AD state A2 can happen later, since Applicants observed brains herein with high scores for both NdStress and Alz, which may be associated with a different path to AD. Alternatively, it is possible that A2 is localized to a brain region not covered in the dataset herein. Thus, this transition may appear later than A1 in a particular brain region and happen much earlier in some other brain region.

This proposed model is most consistent with an age-based hypothesis of Alzheimer's disease that postulates three fundamental steps: 1) an initial injury aggravated by aging, 2) chronic neuroinflammation, and 3) a transition of most brain cells to a new state (Herrup, K. 2010, J. Neurosci., 30: 16755-16762). These key stages of the disease were independently observed and associated with transcriptional changes in Applicants' analysis of brain transcriptome. Applicants herein also identified a striking resemblance of the biological processes behind the disease progression biomarkers and epithelial-to-mesenchymal transition (EMT) (Kalluri, R. and Weinberg, R. A., 2009, J. Clin. Invest., 119:1420:1428). The AD processes are most similar to EMT type 2, which is dependent on inflammation-inducing injuries for initiation and continued occurrence. Associated with tissue regeneration and organ fibrosis in kidney, lung, and liver, EMT type 2 generates mesenchymal cells that produce excessive amounts of extracellular matrix (ECM). Similarly, a transition of AD brain into a tissue enriched with mesenchymal cells produces a large amount of ECM containing β-amyloid. This model of the disease implies that multiple independent genetic factors, as well as infections and/or injuries may accelerate consecutive transitions leading to disease. This also suggests that different therapeutic strategies may be appropriate for early and late disease stages. Therapies targeting lipid metabolism and inflammation may be more effective in the early stages. In the late stages, when the brain becomes enriched in mesenchymal-like signaling and adhesion processes, novel approaches that support the survival of the new state of the brain tissue should be considered.

Projection of Human Aging into Animal Models

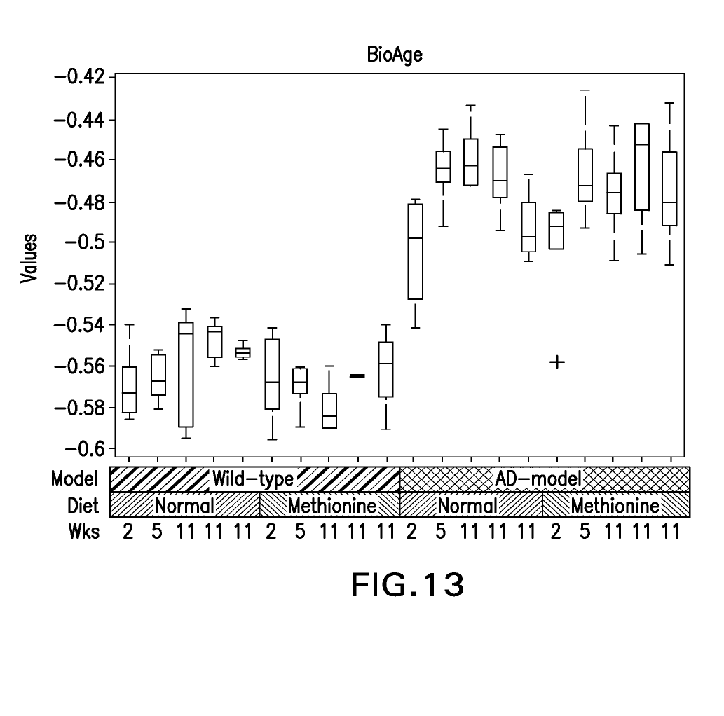

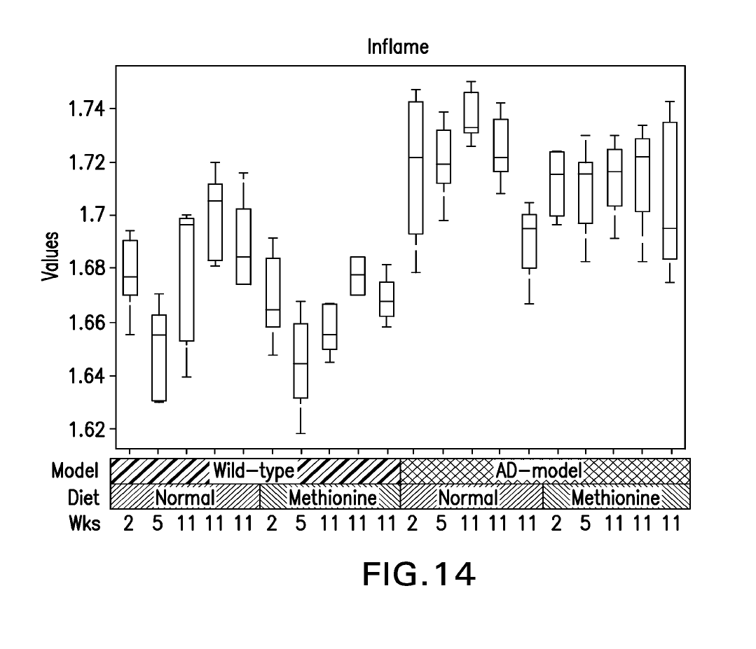

FIGS. 13 and 14 are illustrative of the signature scores for human BioAge and Inflame, respectively. The signature score, i.e. Score, is calculated from groups of genes that are highly correlated. Cell lines and non-human mammals would be evaluated to identify and select a model having a comparable signature score for each of the biomarkers, i.e. BioAge, Inflame, NdStress, and Alz. We used wild-type (C57B) and AD (NFEV) mouse models. The animals were put on a normal and methionine-rich diet (Test Diet, Richmond, Ind.) for 2 to 11 weeks. The increased value of BioAge or Inflame along the y-axis in the AD model with respect to wild type demonstrated that the aging and inflammation processes in AD have progressed further than in normal controls.

### Detection of Brain Signatures in Peripheral Tissues

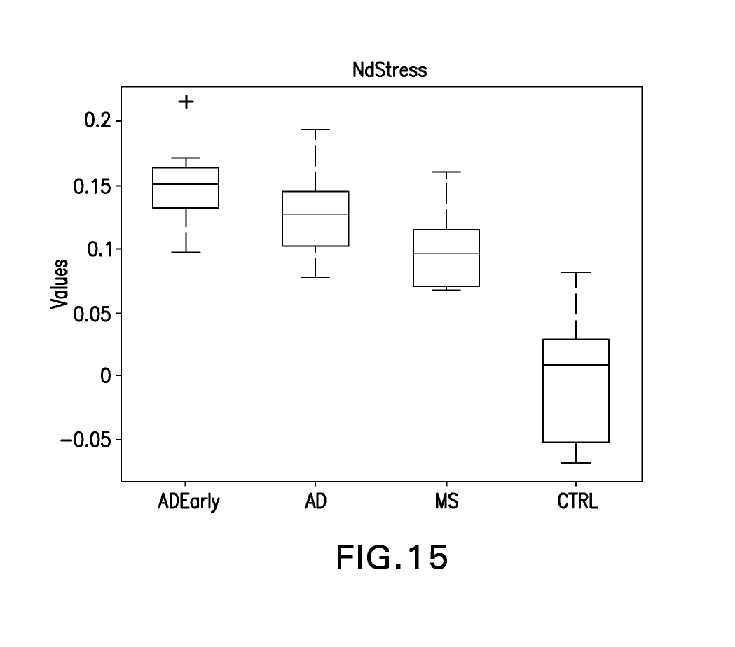

As shown in FIG. 15, the NdStress signature score is elevated in AD-early, AD-late, and MS blood samples relative to those of the controls, i.e. non-demented, normal subjects. Blood samples from seven control (CTRL), eight AD-early, ten AD (late), and nine multiple sclerosis (MS) samples were profiled. The NdStress gene expression score, i.e. gene signature score, was calculated after translating the biomarker gene symbols into human equivalents and matching the probes on a human microarray (Affeymetrix, Santa Clara, Calif.). The NdStress score shows elevated values in subjects with neurodegenerative diseases in comparison to control subjects. This suggests the possibility of using the NdStress biomarker as a peripheral diagnostic tool, that is a biomarker for use with a fluid sample, such as blood, plasma, or CSF.

### EXAMPLES

The following abbreviations are used herein: AD: Alzheimer's disease; ANOVA: ?; AUROC: area under receiver operation characteristics; PFC1: prefrontal cortex from phase 1; PFC2: prefrontal cortex from phase 2; VC1: visual cortex from phase 1; VC2: visual cortex from phase 2; CR1: cerebellum from phase 1; CR2: cerebellum from phase 2; HD: Huntington disease.

### Example 1

### Study Population and Sample Collection

The dataset comprises gene expression data from brain tissue samples that were posthumously collected from more than 600 individuals with diagnosed with Alzheimer's disease (AD), Huntington disease (HD), or with normal, non-demented brains. All brains were obtained from individuals for whom both the donor and the next of kin had completed the Harvard Brain Tissue Resource Center Informed Consent Form (HBTRC, McLean Hospital, Belmont, Mass.). All tissue samples were handled and the research conducted according to the HBTRC Guidelines, including those relating to Human Tissue Handling Risks and Safety Precautions, and in compliance with the Human Tissue Single User Agreement and the HBTRC Acknowledgment Agreement. Table 10 summarizes the composition of the HBTRC gene expression dataset by experimental phase, brain region, gender, and diagnosis at the time of death.

TABLE 10

| | | | | | | | | Mean | | |
|---|---|---|---|---|---|---|---|---|---|---|
| Region, | | | | | Mean | Age | Mean | Braak | Mean | Mean |
| Phase | Diagnosis | Total | Males | Females | Age | Range | PMI | Stage | pH | RIN |
| PFC1 | Normal | 125 | 93 | 32 | 63.8 | 22-106 | 22.2 | 0.6 | 6.4 | 7.2 |
| | Alzheimer | 181 | 81 | 100 | 79.7 | 47-100 | 14.5 | 4.9 | 6.2 | 6.7 |
| VC1 | Normal | 104 | 82 | 22 | 63.5 | 22-106 | 22.4 | 1.5 | 6.4 | 7.0 |
| | Alzheimer | 116 | 57 | 59 | 79.7 | 47-100 | 14.1 | 4.4 | 6.3 | 6.7 |
| CR1 | Normal | 103 | 80 | 23 | 63.3 | 22-106 | 22.0 | 0.5 | 6.5 | 6.6 |
| | Alzheimer | 173 | 79 | 94 | 79.8 | 54-100 | 14.9 | 4.9 | 6.4 | 6.5 |
| PFC2 | Normal | 38 | 30 | 8 | 63.2 | 50-86 | 22.4 | 0.7 | 6.6 | 6.9 |
| | Alzheimer | 115 | 41 | 74 | 81.5 | 59-98 | 12.6 | 4.9 | 6.3 | 6.8 |
| | Huntington | 141 | 74 | 67 | 57.7 | 21-85 | 20.8 | 0.6 | 6.4 | 7.3 |
| VC2 | Normal | 23 | 18 | 5 | 61.0 | 50-80 | 22.2 | 0.8 | 6.5 | 7.0 |
| | Alzheimer | 53 | 18 | 35 | 81.0 | 60-95 | 11.7 | 5.2 | 6.2 | 6.6 |
| | Huntington | 132 | 65 | 67 | 56.5 | 18-93 | 20.6 | 0.4 | 6.4 | 7.0 |
| CR2 | Normal | 25 | 20 | 5 | 63.3 | 50-82 | 22.0 | 0.7 | 6.5 | 6.5 |
| | Alzheimer | 49 | 17 | 32 | 80.1 | 59-97 | 13.5 | 5.0 | 6.5 | 6.4 |
| | Huntington | 139 | 72 | 67 | 56.3 | 18-93 | 20.0 | 0.4 | 6.5 | 6.7 |

The brain regions profiled included dorsolateral prefrontal cortex (PFC, Brodmann area 9), visual cortex (VC, Brodmann area 17), and cerebellum (CR). These regions were chosen because, in AD, the PFC is impacted by the pathology, while the VC and CR regions remain largely intact throughout most of the disease (Braak, 1991). The samples were flash frozen in liquid nitrogen vapor with an average post-mortem interval of about 18 hours. Sample clinical information included age at the time of death (Mean Age and Age Range), gender, Braak stage of AD (Braak, 1991), and pH in different brain tissue samples summarized in Table 10. Braak stage and atrophy were assessed by pathologists at McLean Hospital (Belmont, Mass.). Only neuropathologically confirmed AD subjects with Braak scores >3 were included in this profiling experiment.

### Example 2

### Gene Expression Profiling

The total of 1 μg mRNA from each sample was extracted, amplified to fluorescently labeled tRNA, and profiled by the Rosetta Gene Expression Laboratory in two phases using Rosetta/Merck 44k 1.1 microarray (GPL4372) (Agilent Technikogies, Santa Clara, Calif.) (Hughes, 2001, Nat. Biotechnol., 19:342-347). The average RNA integrity number of 6.81 was sufficiently high for the microarray experiment monitoring 40,638 transcripts representing more than 31,000 unique genes. The expression levels were processed and normalized to the average of all samples in the batch from the same region using Rosetta Resolver (Rosetta Biosoftware, Seattle, Wash.).

Applicants refer to each batch of samples hybridized to the microarrays profiled at the same time by use of the abbreviation for the brain region and the phase of the experiment (e.g., PFC2 refers to prefrontal cortex samples profiled in phase 2). Table 10 summarizes the number of samples in each category. All microarray data generated in this study are available through the National Brain Databank at the Harvard Brain Tissue Resource Center (McLean Hospital, Belmont, Mass.).

### Example 3

### Data Analysis

Applicants used the log 10-ratio of the individual microarray intensities to the average intensities of all samples from the same brain region profiled in the same phase as a primary measure of gene expression. Quality control of gene expression data was performed by principal component analysis using MATLAB R2007a (Mathworks Inc. Natick, Mass.). Outlier samples (less than 2%) were removed from the data set based on extreme standardized values of the first, second, or third principal components, with absolute z-scores more than 3.

The first principal component (PC1) was used to assess the major pattern of gene expression variability in the dataset. Genes that were highly correlated with PC1 were used to build a surrogate biomarker. Throughout this work Applicants used Pearson correlation coefficients, ρ, and assessed their significance, p, assuming normal distribution for Fisher z-transformed values, atanh ρ (Rosner, 2010, Fundamentals of Biostatistics). Significant differential expression for each gene was evaluated using t-test p-values (Rosner, 2010, Fundamentals of Biostatistics, Duxbury Press, Boston Mass.). Multiple testing correction of p-values was done according to Benjamini-Hochberg procedure to obtain false-discovery rates (FDR) (Benjamini and Hochberg, 1995, 57:289-300). These analyses were performed using Statistical Toolbox of MATLAB R2007a (Mathworks Inc. Natick, Mass.).

Gene expression changes associated with aging and disease were characterized by metagenes combining sets of genes with significant association with a disease trait and a very strong Pearson correlation with each other. Applicants utilized a procedure of exploring covariance structure of the gene expression data which was similar to metagene identification (Tamayo, 2007, Proc. Natl. Acad. Sci. U.S.A., 104: 5959-5964), factor analysis of gene expression (Carvalho, 2008, J. Amer. Stat. Assoc., 103: 1438-1456), and supervised gene module discovery (Oldham, 2008, Nat. Neurosci., 11: 1271-1282; Miller, 2008, J. Neurosci., 28: 1410-1420). Instead of genome-wide search for metagenes followed by analysis of associations between metagenes and disease traits, Applicants used a supervised approach. After selecting genes significantly associated with the disease, Applicants agglomeratively clustered them using Pearson correlation as a distance measure. Especially tight and large clusters in the dendrogram were then assigned to biomarkers, i.e. the dendrogram was cut so that several hundred genes in a branch qualified for a biomarker and the average of their correlations to the mean was not weaker than 0.75. Applicants recognized that some signatures could have two anti-correlated arms representing opposite trends in the gene expression (e.g. genes that are up- and down-regulated with the end point).

### Example 4

### Biomarker Scoring

Through out the experiments herein, Applicants utilize the term “biomarker” to refer to a metagene together with its associated score that quantifies it in each brain tissue sample. The biomarker score for each sample was calculated as the mean expression levels of the comprising genes or as the arithmetic difference between the means in the positive and negative arms of the signature when both arms were specified. See, for example, Tables 1-7 that show representative genes making up the biomarkers of the invention herein. Thus, the “Score” was calculated as follows:

$$Score = {〈 log \frac{I}{{I}_{0}} 〉}_{UP} - {〈 log \frac{I}{{I}_{0}} 〉}_{DOWN}$$

where I/I₀ was the normalized intensity of the signature probes. To produce a robust score, all samples have to be normalized to the same reference. The reference intensity I₀ for each gene corresponded to the average intensity in the cohort. The overall coherence of biomarkers was evaluated as an average correlation between individual genes and the average score. Applicants found that averaging coherent genes (coherence >0.75) that correlate with each other produced a measure that was more accurate than for individual genes. For all biomarkers identified in this work, the Score represented a continuous measure of progression for a particular aspect of disease in each sample. To evaluate the performance of the signature score, i.e. Score, as a classifier between diseased and normal samples, Applicants used the area under the curve for the receiver operating characteristic (AUROC) (Hanley, J. A. and McNeil, B. J., 1982, Radiology, 143: 29-36). AUROC is equal to the probability that two randomly selected tissue samples from two groups will be correctly assigned to the correct group based on the relative values of the classifier.

### Example 5

### In Silico Experiments

To validate the biomarkers identified in this work Applicants tested their coherence (mutual correlation between genes) and predictive power (correlation with clinical end points) in the context of an independent gene expression dataset, GSE 1572 (Lu, 2004, Nature, 429:883-891). This data set contained gene expression data from PFC samples of 30 non-demented subject, aged 26-106. These samples were profiled on Human Genome U95 Version 2 Array (GPL8300) (Affymetrix Inc., Santa Clara Calif.). To select the microarray probes and calculate the biomarker score, Applicants matched the biomarker gene symbols to those represented on the HG-U95Av2 array.

An additional set of public gene expression data used to validate the coherence and predictive power of the biomarkers was obtained from hippocampus samples from elderly control and AD subjects, GSE1297 (Blalock, 2004, Proc. Nat. Acad. Sci., USA, 101:2173-2178; Gomez Ravetti, 2010, PlosONE, 5:e10153). These 31 samples were profiled using Affymetrix Human Genome U133A Array (HG-U133A). To select the probes and calculate the biomarker score, Applicants matched the biomarker gene symbols to those represented on the array and averaged the gene expression values according to the equation in the previous subsection.

### Example 6

### Projection of Human Gene Signatures in Animal Models

The human BioAge (FIG. 13) and Inflame (FIG. 14) gene signature scores were projected into a wild type and AD mouse model (NFEV, APP transgenic animal having a mutated β-secretase cleavage site, U.S. Pat. No. 7,432,414) that were fed either a normal or methionine-rich diet (Test Diet, Richmond, Ind.) for a period of 2 to 11 weeks, according to the methods set forth in McCampbell et al., J. Neurochemistry, 2011, 116:82-92, which is incorporated herein in its entirety as if set forth at length.

### Example 7

### Detection of Human Brain Gene Signatures in Peripheral Tissues

For the detection of a human brain gene signature in a peripheral tissue sample, such as blood, Applicants obtained a total of 29 human samples (six normal controls, seven early stage Alzheimer's disease (AD), nine late stage AD, and seven multiple sclerosis (MS)) from PrecisionMed (Solana Beach, Calif.). All subjects were age and gender matched. Alzheimer's disease samples were chosen to have a comparable number of ApoE ε4 carriers and non-carriers. Samples were amplified using a standard amplification kit (NuGEN Technologies, Inc., San Carlos, Calif.) and profiled using a standard microarray (Affymetrix, Santa Clara, Calif.) according to the manufacturer's protocols.

What is claimed:

1. A biomarker comprising a set of one or more correlated genes, having a gene signature score that is significantly different between groups of tissue samples according to a statistical test, wherein the signature score is equivalent to the average gene expression of the up-regulated genes for said marker minus the average gene expression of the down-regulated genes.

2. A biomarker of claim 1 selected from the group consisting of BioAge, Inflame, NdStress, and Alz.

3. The biomarker of claim 2 comprising a set of one or more correlated genes listed in Tables 1-7.

4. A non-human transgenic mammal having the biomarker of claim 1 for use in evaluating the disease progression of Alzheimer's disease.

5. The non-human transgenic mammal of claim 4 for use in evaluating a therapeutic for the prevention or treatment of Alzheimer's disease.

6. The biomarker of claim 1 for use in evaluating the disease progression of Alzheimer's disease in a peripheral tissue sample.

7. The biomarker of claim 1 for use in evaluating a therapeutic for the prevention or treatment of Alzheimer's disease in a peripheral tissue sample.
